(19) United States
(12) Patent Application Publication
(10) Pub. No.: US 2023/0271964 A1
(43) Pub. Date: Aug. 31, 2023

(54) 5-AMINO-8-(4-PYRIDYL)-[1,2,4]TRIAZOLO[4,3-C]PYRIMIDIN-3-ONE COMPOUNDS FOR USE AGAINST CANCER

(71) Applicant: ASTRAZENECA AB, Södertälje (SE)

(72) Inventors: Giles Albert Brown, Cambridge (GB); Christine Mary Richardson, Saffron Walden (GB); Miles Stuart Congreve, Cambridge (GB); Rebecca Paul, St. Albans (GB); Stephen Philippe Andrews, Royston (GB); Jonathan Stephen Mason, Cambridge (GB)

(21) Appl. No.: 17/914,245

(22) PCT Filed: Mar. 25, 2021
(86) PCT No.: PCT/EP2021/057806

§ 371 Date: Sep. 23, 2022

Related U.S. Application Data
(60) Provisional application No. 62/994,908, filed on Mar. 26, 2020.

(51) Int. Cl. C07D 487/04

(52) U.S. Cl. CPC C07D 487/04; C07B 2200/05

(57) ABSTRACT

Described herein are triazalone compounds of Formula (I) and pharmaceutically acceptable salts thereof. Methods of making and using compounds of Formula (I) are also described. Compounds of Formula (I) and pharmaceutically acceptable salts thereof can be useful as adenosine receptor antagonists, for example in the treatment of diseases or conditions mediated by the adenosine receptor, such as cancer, movement disorders, or attention disorders.

### BACKGROUND

Adenosine modulates of a number of physiological functions. Intracellularly, adenosine is involved in energy metabolism, nucleic acid metabolism, and the methionine cycle; extracellular adenosine engages in intercellular signaling. For example, extracellular adenosine is a potent immunosuppressor, preventing an overzealous immune response during inflammation and infection. Adenosine also acts on other systems, including the cardiovascular system, and the central nervous system.

The action of adenosine is mediated by a family of G-protein coupled receptors. At least four subtypes of adenosine receptors have been identified: A1R, A2aR, A2bR, and A3R. The A1R and A3 subtypes inhibit the activity of the enzyme adenylate cyclase, whereas the A2a and A2b subtypes stimulate the activity of the same enzyme, thereby modulating the level of cyclic AMP in cells.

In the immune system, engagement of A2a and A2b adenosine receptors is a critical regulatory mechanism that protects tissues against excessive immune reactions. In tumors, this pathway is hijacked and hinders antitumor immunity, promoting cancer progression. Furthermore, in many cases, the tumor microenvironment contains high levels of extracellular adenosine. Thus, the adenosine receptor, notably A2aR and A2bR, have been identified as targets for cancer therapies.

Numerous adenosine receptor antagonists have been reported. For example, International Patent Application WO 2006/138734 discloses triazolopyrimidine cannabinoid receptor 1 (CB-1) antagonists. WO 2008/002596 and WO 2009/111449 disclose adenosine A2a receptor antagonists which include a triazolone moiety. WO 2012/038980 discloses fused tricyclic compounds as adenosine receptor antagonists. WO 2016/161282 discloses heterocyclic compounds as LSD1 inhibitors. WO 2018/166493 discloses heteroaryl[4,3-c]pyrimidine-5-amine derivatives for use as A2a receptor antagonists.

There remains a need for adenosine receptor antagonists that are highly soluble, highly selective, and highly potent.

### SUMMARY

In one aspect, a compound of Formula (I):

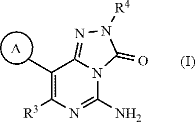

or a pharmaceutically acceptable salt thereof, is provided, wherein:
ring A can be:

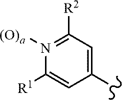

;
each R¹ and each R², independently, can be halo, C₁₋₃alkyl, -O-C₁₋₃alkyl, —CO₂R^{a}, or —NR⁷R⁸;
   wherein alkyl is optionally substituted with one or more substituents independently
   selected from —OR^{a} and halo;
R³ can be C₁₋₆alkyl, C₂₋₆alkenyl, C₂₋₆alkynyl, aryl, heterocyclyl, heteroaryl, halo, —OR^{a}, —NR^{a}R^{b}, —CO₂R^{a}, —CONR^{a}R^{b}, —NR^{a}C(O)—R^{a}, or —NHC(O)—OR^{a};
   wherein heterocyclyl and heteroaryl, independently, include from 1 to 4 heteroatoms independently selected from N, O, and S(O)ₖ;
   wherein R³ is optionally substituted with from one to three substituents selected from halo, cyano, —R^{a}, and —OR^{a};
R⁴ can be —(CHR^{c})ᵢ—(NR^{a})ⱼ—R⁵;
R⁵ can be a 5-membered heterocyclyl or 5-membered heteroaryl, each including from 1 to 4 heteroatoms independently selected from N, O, and S(O)ₖ;
   wherein one or two ring atoms of R⁵ is optionally replaced by —C(═O)—;
   wherein R⁵ is optionally substituted with from one to four groups —X—R⁶;
each X, independently, can be a bond, —O—, —NR^{a}—, —S(O)ₖ—, —(CH₂)ₘ—, or —C(O)—;
each R⁶, independently, can be H, halo, —OR^{a}, C₁₋₆alkyl, C₃₋₈cycloalkyl, heterocyclyl, heteroaryl, aryl, —CO₂R^{a}, —C(O)NR^{a}R^{b}, —(CH₂)ₙ—NR^{a}R^{b}, or cyano;
   wherein each of heterocyclyl and heteroaryl includes from 1 to 4 heteroatoms
   independently selected from N, O, and S(O)ₖ;
   wherein one or two ring atoms of each C₃₋₈cycloalkyl, heterocyclyl, heteroaryl, or aryl, independently is optionally replaced by —C(═O)—;
   wherein each of alkyl, cycloalkyl, heterocyclyl, heteroaryl, and aryl is optionally substituted with one or more substituents independently selected from —R^{a}, —OR^{a}, —(CH₂)ₙ—NR^{a}R^{b}, and halo;
each R⁷ and each R⁸, independently, can be R^{a};
or R⁷ and R⁸ together with the atom to which they are attached can form a 3- to 8-membered heterocyclyl optionally substituted with one or more substituents independently selected from —OR^{a} and halo;
each R^{a} and each R^{b}, independently, can be H, C₁₋₆alkyl, C₃₋₈cycloalkyl, or C₄₋₉cycloalkylalkyl;
   wherein each R^{a} and each R^{b}, independently, is optionally substituted with one or more
   substituents independently selected from —OH and halo;
each R^{c}, independently, can be H, halo, C₁₋₃alkyl, or —(CH₂)ₙ—NR^{a}R^{b};
   wherein alkyl is optionally substituted with one or more substituents independently
   selected from —OR^{a} and halo;
a can be 0 or 1;
i can be 0, 1, 2, or 3;
j can be 0 or 1;
each k, independently, can be 0, 1, or 2;
each m, independently, can be 1 or 2; and
each n, independently, can be 0 or 1.

The compound of Formula (I) can be a selective adenosine receptor antagonist with respect to CB-1. The compound can have a Ki for at least one of A2aR and A2bR of 100 nM or less, and can have a Ki for CB-1 of 10,000 nM or more.

In some embodiments, i can be 1 and R^{c} can be H or C₁₋₃ alkyl. R⁵ can be selected from imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups -X-R⁶. X can be a bond and R⁶ can be C₁₋₆alkyl.

In some embodiments, R³ can be phenyl optionally substituted with fluoro or chloro. R¹ and R² can be each independently selected from halo, —CH₃, —CH₂OH, or —OCH₃.

In another aspect, a compound of Formula (II):

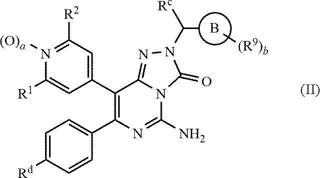

or a pharmaceutically acceptable salt thereof, is provided, wherein:
each R¹ and each R², independently, can be halo, C₁₋₃alkyl, or -O-C₁₋₃alkyl;
   wherein alkyl is optionally substituted with one or more substituents independently
   selected from —OH and halo;
ring B can be a 5-membered heterocyclyl or 5-membered heteroaryl, each including from 1 to 4 heteroatoms independently selected from N and O;
each R⁹, independently, can be halo or C₁₋₃alkyl;
   wherein alkyl is optionally substituted with one or more substituents independently
   selected from —OH and halo;
each R^{a} and each R^{b}, independently, can be H, C₁₋₆alkyl, C₃₋₈cycloalkyl, or C₄₋₉cycloalkylalkyl;
   wherein each R^{a} and each R^{b}, independently, is optionally substituted with one or more
   substituents independently selected from —OH and halo;
R^{c} can be H, halo, C₁₋₃alkyl, or —(CH₂)ₙ—NR^{a}R^{b};
   wherein alkyl is optionally substituted with one or more substituents independently
   selected from —OR^{a} and halo;
R^{d} can be H or halo;
a can be 0 or 1;
b can be 0, 1, or 2; and
n can be 0 or 1.

In some embodiments ring B can be tetrahydrofuranyl or 1,3-oxazolyl, each of which is optionally substituted with 1 to 3 substituents selected from -C₁₋₃alkyl.

In another aspect, a compound, or a pharmaceutically acceptable salt thereof, is provided, wherein the compound is selected from the group consisting of:
5-amino-8-(2,6-dimethyl-4-pyridyl)-7-phenyl-2-(2-pyrazol-1-ylethyl)-[1,2,4]triazolo[4,3-c]pyrimidin-3-one;
5-amino-8-(2,6-dimethyl-4-pyridyl)-2-[2-(1-methylimidazol-2-yl)ethyl]-7-phenyl-[1,2,4]triazolo[4,3-c]pyrimidin-3-one;
5-amino-8-(2,6-dimethyl-4-pyridyl)-2-[2-(1H-imidazol-2-yl)ethyl]-7-phenyl-[1,2,4]triazolo[4,3-c]pyrimidin-3-one;
5-amino-8-(2,6-dimethyl-4-pyridyl)-7-phenyl-2-[2-(1H-tetrazol-5-yl)ethyl]-[1,2,4]triazolo[4,3-c]pyrimidin-3-one;
5-amino-8-(2,6-dimethyl-4-pyridyl)-2-[2-(1-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one;
5-amino-8-(2,6-dimethyl-4-pyridyl)-7-phenyl-2-[1-(2-thienyl)ethyl]-[1,2,4]triazolo[4,3-c]pyrimidin-3-one;
5-amino-8-(2,6-dimethyl-4-pyridyl)-2-(oxazol-2-ylmethyl)-7-phenyl-[1,2,4]triazolo[4,3-c]pyrimidin-3-one;
5-amino-8-(2,6-dimethyl-4-pyridyl)-2-(oxazol-4-ylmethyl)-7-phenyl-[1,2,4]triazolo[4,3-c]pyrimidin-3-one;
5-amino-8-(2,6-dimethyl-4-pyridyl)-2-[(1-methylimidazol-2-yl)methyl]-7-phenyl-[1,2,4]triazolo[4,3-c]pyrimidin-3-one;
5-amino-8-(2,6-dimethyl-4-pyridyl)-2-[(2-methylpyrazol-3-yl)methyl]-7-phenyl-[1,2,4]triazolo[4,3-c]pyrimidin-3-one;
5-amino-8-(2,6-dimethyl-4-pyridyl)-2-(isoxazol-3-ylmethyl)-7-phenyl-[1,2,4]triazolo[4,3-c]pyrimidin-3-one;
5-amino-8-(2,6-dimethyl-4-pyridyl)-2-[(5-methylisoxazol-3-yl)methyl]-7-phenyl-[1,2,4]triazolo[4,3-c]pyrimidin-3-one;
5-amino-8-(2,6-dimethyl-4-pyridyl)-2-[(2-methyloxazol-4-yl)methyl]-7-phenyl-[1,2,4]triazolo[4,3-c]pyrimidin-3-one;
5-amino-8-(2,6-dimethyl-4-pyridyl)-2-[(1-methylpyrazol-3-yl)methyl]-7-phenyl-[1,2,4]triazolo[4,3-c]pyrimidin-3-one;
5-amino-8-(2,6-dimethyl-4-pyridyl)-2-[(5-methyl-1,3,4-oxadiazol-2-yl)methyl]-7-phenyl-[1,2,4]triazolo[4,3-c]pyrimidin-3-one;
5-amino-8-(2,6-dimethyl-4-pyridyl)-2-[(3-methylimidazol-4-yl)methyl]-7-phenyl-[1,2,4]triazolo[4,3-c]pyrimidin-3-one;
5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one;
5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one;
5-amino-8-(2,6-dimethyl-4-pyridyl)-2-[(3-methyl-1,2,4-oxadiazol-5-yl)methyl]-7-phenyl-[1,2,4]triazolo[4,3-c]pyrimidin-3-one;
5-amino-8-(2,6-dimethyl-4-pyridyl)-2-[(5-methyloxazol-4-yl)methyl]-7-phenyl-[1,2,4]triazolo[4,3-c]pyrimidin-3-one;
5-amino-8-(2,6-dimethyl-4-pyridyl)-2-(1H-imidazol-5-ylmethyl)-7-phenyl-[1,2,4]triazolo[4,3-c]pyrimidin-3-one;
5-amino-8-(2,6-dimethyl-4-pyridyl)-2-(1H-imidazol-2-ylmethyl)-7-phenyl-[1,2,4]triazolo[4,3-c]pyrimidin-3-one;
5-amino-8-(2,6-dimethyl-4-pyridyl)-7-phenyl-2-(1H-pyrazol-5-ylmethyl)-[1,2,4]triazolo[4,3-c]pyrimidin-3-one;
5-amino-8-(2,6-dimethyl-4-pyridyl)-7-phenyl-2-(2H-tetrazol-5-ylmethyl)-[1,2,4]triazolo[4,3-c]pyrimidin-3-one;
5-amino-8-(2,6-dimethyl-4-pyridyl)-2-(1,3,4-oxadiazol-2-ylmethyl)-7-phenyl-[1,2,4]triazolo[4,3-c]pyrimidin-3-one;
5-amino-8-(2,6-dimethyl-4-pyridyl)-2-[(4-methyl-1,2,4-triazol-3-yl)methyl]-7-phenyl-[1,2,4]triazolo[4,3-c]pyrimidin-3-one;
5-amino-8-(2,6-dimethyl-4-pyridyl)-2-[(5-methyl-1H-triazol-4-yl)methyl]-7-phenyl-[1,2,4]triazolo[4,3-c]pyrimidin-3-one;
5-amino-8-(2,6-dimethyl-4-pyridyl)-2-[(2-methyltriazol-4-yl)methyl]-7-phenyl-[1,2,4]triazolo[4,3-c]pyrimidin-3-one;
5-amino-8-(2,6-dimethyl-4-pyridyl)-7-(4-fluorophenyl)-2-[(5-methyloxazol-4-yl)methyl]-[1,2,4]triazolo[4,3-c]pyrimidin-3-one;
5-amino-2-[(2,5-dimethyloxazol-4-yl)methyl]-8-(2,6-dimethyl-4-pyridyl)-7-(4-fluorophenyl)-[1,2,4]triazolo[4,3-c]pyrimidin-3-one;
5-amino-2-[[1-benzyl-3-(3-methoxyphenyl)pyrazol-4-yl]methyl]-8-(2,6-dimethyl-4-pyridyl)-7-phenyl-[1,2,4]triazolo[4,3-c]pyrimidin-3-one;
5-amino-8-(2,6-dimethyl-1-oxido-pyridin-1-ium-4-yl)-2-[(5-methyloxazol-4-yl)methyl]-7-phenyl-[1,2,4]triazolo[4,3-c]pyrimidin-3-one;
5-amino-2-[(2,5-dimethyloxazol-4-yl)methyl]-8-(2,6-dimethyl-1-oxido-pyridin-1-ium-4-yl)-7-(4-fluorophenyl)-[1,2,4]triazolo[4,3-c]pyrimidin-3-one;
5-amino-8-(2,6-dimethyl-1-oxido-pyridin-1-ium-4-yl)-2-[(1-methylimidazol-2-yl)methyl]-7-phenyl-[1,2,4]triazolo[4,3-c]pyrimidin-3-one;
5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one;
5-amino-8-(2-methoxy-6-methyl-4-pyridyl)-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-2-ylmethyl)-7-phenyl-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-4-ylmethyl)-7-phenyl-[1,2,4]triazolo[4,3-c]pyrimidin-3-one;
5-amino-8-[2-(hydroxymethyl)-6-methyl-4-pyridyl]-2-[(5-methylisoxazol-3-yl)methyl]-7-phenyl-[1,2,4]triazolo[4,3-c]pyrimidin-3-one;
5-amino-2-[(3,5-dimethylimidazol-4-yl)methyl]-8-[2-(hydroxymethyl)-6-methyl-4-pyridyl]-7-phenyl-[1,2,4]triazolo[4,3-c]pyrimidin-3-one;
5-amino-8-[2-(hydroxymethyl)-6-methyl-4-pyridyl]-2-[(1-methylpyrazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-(oxazol-4-ylmethyl)-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methyl-4-pyridyl]-2-[(1-methylimidazol-2-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(1-methylpyrazol-3-yl)methyl]-[1,2,4]triazolo[4,3-c]pyrimidin-3-one;
5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-7-(4-fluorophenyl)-2-[(1-methylimidazol-2-yl)methyl]-[1,2,4]triazolo[4,3-c]pyrimidin-3-one;
5-amino-8-[2-chloro-6-(hydroxymethyl)-4-pyridyl]-7-(4-fluorophenyl)-2-[(5-methyloxazol-4-yl)methyl]-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methoxy-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methoxy-4-pyridyl]-2-[(1-methylimidazol-2-yl)methyl]-[1,2,4]triazolo[4,3-c]pyrimidin-3-one;
5-amino-8-[2-(hydroxymethyl)-6-(trifluoromethyl)-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-(trifluoromethyl)-4-pyridyl]-2-[(1-methylimidazol-2-yl)methyl]-7-phenyl-[1,2,4]triazolo[4,3-c]pyrimidin-3-one;
tert-butyl 3-[5-amino-8-(2,6-dimethyl-4-pyridyl)-3-oxo-7-phenyl-[1,2,4]triazolo[4,3-c]pyrimidin-2-yl]pyrrolidine-1-carboxylate;
5-amino-8-(2,6-dimethyl-4-pyridyl)-7-phenyl-2-pyrrolidin-3-yl-[1,2,4]triazolo[4,3-c]pyrimidin-3-one;
5-amino-8-(2,6-dimethyl-4-pyridyl)-2-(1-methylpyrrolidin-3-yl)-7-phenyl-[1,2,4]triazolo[4,3-c]pyrimidin-3-one;
tert-butyl 2-[[5-amino-8-(2,6-dimethyl-4-pyridyl)-3-oxo-7-phenyl-[1,2,4]triazolo[4,3-c]pyrimidin-2-yl]methyl]pyrrolidine-1-carboxylate;
5-amino-8-(2,6-dimethyl-4-pyridyl)-7-phenyl-2-(pyrrolidin-2-ylmethyl)-[1,2,4]triazolo[4,3-c]pyrimidin-3-one;
5-amino-8-(2,6-dimethyl-4-pyridyl)-2-[(1-methylpyrrolidin-2-yl)methyl]-7-phenyl-[1,2,4]triazolo[4,3-c]pyrimidin-3-one;
5-amino-8-(2,6-dimethyl-4-pyridyl)-2-[[(2R)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-(2-methoxyethyl)pyrrolidin-2-yl]methyl]-7-phenyl-[1,2,4]triazolo[4,3-c]pyrimidin-3-one;
5-amino-8-(2,6-dimethyl-4-pyridyl)-2-[[(2R)-1-(2-methoxyethyl)pyrrolidin-2-yl]methyl]-7-phenyl-[1,2,4]triazolo[4,3-c]pyrimidin-3-one;
5-amino-2-[[(2S)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2S)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one;
5-amino-8-(2,6-dimethyl-4-pyridyl)-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[[(2S)-tetrahydrofuran-2-yl]methyl]-[1,2,4]triazolo[4,3-c]pyrimidin-3-one;
5-amino-8-(2,6-dimethyl-4-pyridyl)-2-[[(2R,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-7-phenyl-2-(2-pyrrolidin-1-ylethyl)-[1,2,4]triazolo[4,3-c]pyrimidin-3-one;
5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2S)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methyl-4-pyridyl]-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-phenyl-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(4-fluorophenyl)-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methoxy-4-pyridyl]-2-[[(2R)-tetrahydrofuran-2-yl]methyl]-[1,2,4]triazolo[4,3-c]pyrimidin-3-one;
5-amino-8-[2-(hydroxymethyl)-6-(trifluoromethyl)-4-pyridyl]-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one;
5-amino-8-(2,6-dimethyl-4-pyridyl)-7-phenyl-2-[2-[[1-(pyridine-3-carbonyl)pyrrolidin-3-yl] amino] ethyl]-[1,2,4] triazolo [4,3-c]pyrimidin-3-one;
5-amino-8-(2,6-dimethyl-4-pyridyl)-2-[2-[methyl(1H-pyrazol-4-yl)amino]ethyl]-7-phenyl-[1,2,4]triazolo[4,3-c]pyrimidin-3-one;
5-amino-2-[[1-[[2-(aminomethyl)phenyl]methyl]pyrrolidin-2-yl]methyl]-8-(2,6-dimethyl-4-pyridyl)-7-phenyl-[1,2,4]triazolo[4,3-c]pyrimidin-3-one; and
5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one.

In some embodiments, the compound, or pharmaceutically acceptable salt thereof, can be selected from the group consisting of:
5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one; and
5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one.

In another aspect, a pharmaceutical composition comprising a compound of Formula (I) or Formula (II), or a pharmaceutically acceptable salt thereof, and a pharmaceutically acceptable carrier, diluent, or excipient, is provided.

In another aspect, the use of a compound of Formula (I) or Formula (II), or a pharmaceutically acceptable salt thereof, for the treatment of a disease or condition mediated by the adenosine receptor is provided.

In some embodiments, the disease or condition mediated by the adenosine receptor is lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor.

Other features, objects, and advantages will be apparent from the description and from the claims.

### DESCRIPTION

Compounds of Formula (I) and Formula (II), or pharmaceutically acceptable salts thereof, are useful as adenosine receptor antagonists.

A compound of Formula (I):

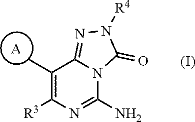

or a pharmaceutically acceptable salt thereof, is described herein.

Ring A is:

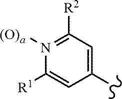

Each R¹ and each R², independently, is halo, C₁₋₃alkyl, -O-C₁₋₃alkyl, —CO₂R^{a}, or —NR⁷R⁸; wherein alkyl is optionally substituted with one or more substituents independently selected from —OR^{a} and halo.

R³ is C₁₋₆alkyl, C₂₋₆alkenyl, C₂₋₆alkynyl, aryl, heterocyclyl, heteroaryl, halo, —OR^{a}, —NR^{a}R^{b}, —CO₂R^{a}, —CONR^{a}R^{b}, —NR^{a}C(O)—R^{a}, or —NHC(O)—OR^{a}; wherein heterocyclyl and heteroaryl, independently, include from 1 to 4 heteroatoms independently selected from N, O, and S(O)ₖ; wherein R³ is optionally substituted with from one to three substituents selected from halo, cyano, —R^{a}, and —OR^{a}.

R⁴ is —(CHR^{c})ᵢ₋(NR^{a})ⱼ—R⁵.

R⁵ is a 5-membered heterocyclyl or 5-membered heteroaryl, each including from 1 to 4 heteroatoms independently selected from N, O, and S(O)ₖ; wherein one or two ring atoms of R⁵ is optionally replaced by —C(═O)—; wherein R⁵ is optionally substituted with from one to four groups —X—R⁶.

Each X, independently, is a bond, —O—, —NR^{a}—, —S(O)ₖ—, —(CH₂)ₘ—, or —C(O)—.

Each R⁶, independently, is H, halo, —OR^{a}, C₁₋₆alkyl, C₃₋₈cycloalkyl, heterocyclyl, heteroaryl, aryl, —CO₂R^{a}, —C(O)NR^{a}R^{b}, —(CH₂)ₙ—NR^{a}R^{b}, or cyano; wherein each of heterocyclyl and heteroaryl includes from 1 to 4 heteroatoms independently selected from N, O, and S(O)ₖ; wherein one or two ring atoms of each C₃₋₈cycloalkyl, heterocyclyl, heteroaryl, or aryl, independently is optionally replaced by —C(═O)—; wherein each of alkyl, cycloalkyl, heterocyclyl, heteroaryl, and aryl is optionally substituted with one or more substituents independently selected from —R^{a}, —OR^{a}, —(CH₂)ₙ—NR^{a}R^{b}, and halo.

Each R⁷ and each R⁸, independently, is R^{a};or R⁷ and R⁸ together with the atom to which they are attached form a 3- to 8-membered heterocyclyl optionally substituted with one or more substituents independently selected from —OR^{a} and halo.

Each R^{a} and each R^{b}, independently, is H, C₁₋₆alkyl, C₃₋₈cycloalkyl, or C₄₋₉cycloalkylalkyl; wherein each R^{a} and each R^{b}, independently, is optionally substituted with one or more substituents independently selected from —OH and halo.

Each R^{c}, independently, is H, halo, C₁₋₃alkyl, or —(CH₂)ₙ—NR^{a}R^{b}; wherein alkyl is optionally substituted with one or more substituents independently selected from —OR^{a} and halo.

a is 0 or 1.

i is 0, 1, 2, or 3.

j is 0 or 1.

Each k, independently, is 0, 1, or 2.

Each m, independently, is 1 or 2.

Each n, independently, is 0 or 1.

In some embodiments, i is 1 and R^{c} is H or C₁₋₃ alkyl.

In some embodiments, R⁵ is 5-membered heterocyclyl. In other embodiments, R⁵ is 5-membered heteroaryl.

In some embodiments, R⁵ is selected from imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups —X—R⁶.

In some embodiments, R⁵ is tetrahydrofuranyl or 1,3-oxazolyl, each of which is optionally substituted with —CH₃.

In some embodiments, i is 1; R^{c} is H; j is 0; and R⁵ is selected from 1,3-oxazolyl and tetrahydrofuranyl, each of which is optionally substituted with —CH₃.

In some embodiments, R¹ and R² are each independently —CH₃ or —CH₂OH; i is 1; R^{c} is H; j is 0; and R⁵ is tetrahydrofuranyl or 1,3-oxazolyl, each of which is optionally substituted with —CH₃.

In some embodiments, X is a bond and R⁶ is C₁₋₆alkyl.

In some embodiments, R³ is phenyl optionally substituted with fluoro or chloro.

In some embodiments, R¹ and R² are each independently selected from halo, —CH₃, —CH₂OH, or —OCH₃.

In some embodiments, R¹ and R² are each independently selected from halo, —CH₃, —CH₂OH, or —OCH₃; and R³ is phenyl optionally substituted with fluoro or chloro.

In some embodiments, R¹ and R² are each independently selected from halo, —CH₃, —CH₂OH, or —OCH₃; R³ is phenyl optionally substituted with fluoro or chloro; i is 1; and R^{c} is H.

In some embodiments, R¹ and R² are each independently —CH₃ or —CH₂OH; R³ is phenyl optionally substituted with fluoro or chloro; i is 1; R^{c} is H; j is 0; and R⁵ is tetrahydrofuranyl or 1,3-oxazolyl, each of which is optionally substituted with —CH₃.

In some embodiments, R³ is phenyl optionally substituted with fluoro or chloro; i is 1; R^{c} is H or C₁₋₃alkyl; j is 0; and R⁵ is selected from imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups —X—R⁶.

In some embodiments, R³ is phenyl optionally substituted with fluoro or chloro; i is 1; R^{c} is H or C₁₋₃alkyl; j is 0; and R⁵ is selected from imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups —X—R⁶, wherein X is a bond and R⁶ is C₁₋₆alkyl.

In some embodiments, R³ is phenyl optionally substituted with fluoro or chloro; i is 1; R^{c} is H or C₁₋₃alkyl; j is 0; and R⁵ is tetrahydrofuranyl or 1,3-oxazolyl, each of which is optionally substituted with —CH₃.

A compound of Formula (II):

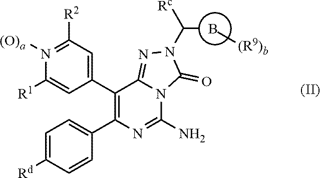

or a pharmaceutically acceptable salt thereof, is provided. Compounds of Formula (II) are encompassed by the broader Formula (I).

Each R¹ and each R², independently, is halo, C₁₋₃alkyl, or -O-C₁₋₃alkyl; wherein alkyl is optionally substituted with one or more substituents independently selected from —OH and halo.

Ring B is a 5-membered heterocyclyl or 5-membered heteroaryl, each including from 1 to 4 heteroatoms independently selected from N and O.

Each R⁹, independently, is halo or C₁₋₃alkyl; wherein alkyl is optionally substituted with one or more substituents independently selected from —OH and halo.

Each R^{a} and each R^{b}, independently, is H, C₁₋₆alkyl, C₃₋₈cycloalkyl, or C₄₋₉cycloalkylalkyl; wherein each R^{a} and each R^{b}, independently, is optionally substituted with one or more substituents independently selected from —OH and halo.

R^{c} is H, halo, C₁₋₃alkyl, or —(CH₂)ₙ—NR^{a}R^{b}; wherein alkyl is optionally substituted with one or more substituents independently selected from —OR^{a} and halo.

R^{d} is H or halo.

a is 0 or 1.

b is 0, 1, or 2.

n is 0 or 1.

In some embodiments, R^{d} is halo.

In some embodiments, R¹ and R² are each independently selected from halo, —CH₃, —CH₂OH, or —OCH₃.

In some embodiments, R¹ and R² are each independently —CH₃ or —CH₂OH.

In some embodiments, ring B is imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups —X—R⁶.

In some embodiments, ring B is tetrahydrofuranyl or 1,3-oxazolyl, b is 0 or 1, and each R⁹, independently, is C₁₋₃alkyl.

In some embodiments, ring B is tetrahydrofuranyl.

In some embodiments, ring B is 1,3-oxazolyl.

In some embodiments, R¹ and R² are each independently —CH₃ or —CH₂OH; ring B is tetrahydrofuranyl or 1,3-oxazolyl; b is 0 or 1; and R^{c} is H.

The term “halo” refers to fluoro, chloro, bromo and iodo.

The term “alkyl” refers to a fully saturated straight-chain or branched aliphatic group, having the number of carbon atoms specified, if designated (e.g., C₁₋₁₀alkyl refers to an alkyl group having one to ten carbons). Examples include as methyl, ethyl, n-propyl, isopropyl, n-butyl, t-butyl, isobutyl, sec-butyl, n-pentyl, n-hexyl, n-heptyl, n-octyl, and the like. If no size is designated, “alkyl” refers to a group having from 1 to 10 carbon atoms.

The term “alkenyl” refers to an unsaturated straight-chain or branched aliphatic group, which contain at least one carbon-carbon double bond, and having the number of carbon atoms specified, if designated. Examples of alkenyl groups include, but are not limited to, vinyl, allyl, 1-propenyl, 2-butenyl, 3-butenyl, 3-methylbut-1-enyl, 1-pentenyl and 4-hexenyl. If no size is designated, “alkenyl” refers to a group having from 2 to 10 carbon atoms.

The term “alkynyl” refers to an unsaturated straight-chain or branched aliphatic group, which contain at least one carbon-carbon triple bond, and having the number of carbon atoms specified, if designated. Examples of alkynyl groups include, but are not limited to, ethynyl, propargyl, and but-2-ynyl. If no size is designated, “alkynyl” refers to a group having from 2 to 10 carbon atoms.

Alkenyl and alkynyl groups can contain more than one unsaturated bond, or a mixture of double and triple bonds.

The term “cycloalkyl” refers to a saturated or unsaturated aliphatic ring containing from 3 to 10 carbon ring atoms, where one or more carbon ring atoms can optionally be replaced by —C(═O)—. A cycloalkyl group can contain fused and/or bridged rings, including where the fused or bridged ring(s) are cycloalkyl. Suitable examples of “cycloalkyl” include, but are not limited to, cyclopropyl, cyclopentyl, cyclobutyl, cyclohexyl, cyclohexenyl, cyclohexynyl, cycloheptyl, norbornyl, 4-oxocyclohex-1-yl and 3-oxocyclohept-5-en-1-yl.

The term “heterocyclyl” refers to a saturated or unsaturated heterocyclic ring containing from 3 to 10 ring atoms, where from 1 to 4 ring atoms are independently N, O, or S; and one or more carbon ring atoms can optionally be replaced by —C(═O)—. A ring nitrogen or a ring sulfur atom, independently, can optionally be oxidized, including for example —N(O)—, —S(O)—, or —S(O)₂—. A ring nitrogen atom in a heterocyclyl group can optionally be quaternized, for example, —N⁺(CH₃)₂—. A heterocyclyl group can contain fused and/or bridged rings, including where the fused or bridged ring(s) are cycloalkyl or heterocyclyl groups. Examples of heterocyclic groups include, but are not limited to, pyrrolidinyl, piperidinyl, piperazinyl, tetrahydrofuranyl, morpholinyl, thiomorphonlinyl, dihydropyranyl, dihydropyridinyl, tetrahydropyranyl, octahydroquinolinyl, octahydroindolizinyl, and decahydroquinolinyl.

The term “aryl” refers to a monocyclic, bicyclic or tricyclic aromatic hydrocarbon group containing from 6 to 14 ring atoms. Aryl may contain fused rings, including aryl rings fused to cycloalkyl, heterocyclyl, or aryl rings. Examples of aryl groups include, but are not limited to, phenyl, naphthyl, anthracenyl, tetrahydronaphthyl, and dihydro-1H-indenyl.

The term “heteroaryl” refers to a monocyclic, bicyclic or tricyclic aromatic group containing from 6 to 14 ring atoms, where from 1 to 4 ring atoms are independently N, O, or S. A ring nitrogen or a ring sulfur atom, independently, can optionally be oxidized, including for example —N(O)—, —S(O)—, or —S(O)₂—. A heteroaryl group can contain fused and/or bridged rings, including where the fused or bridged ring(s) are cycloalkyl, heterocyclyl, aryl, or heteroaryl groups. Examples of heteroaryl groups include, but are not limited to, pyrrolyl, furanyl, pyridyl, imidazolyl, oxazolyl, thiazolyl, pyrimidinyl, 5,6,7,8-tetrahydroquinolinyl, benzofuranyl, pyrrolopyridinyl, pyrrolopyrimidinyl, triazinyl, and tetrazolyl.

The term “multicyclic ring system” refers to a cycloalkyl, heterocyclyl, aryl, or heteroaryl group which includes two or more fused and/or bridged rings.

Some compounds described herein can exist in more than one stereoisomeric form. Descriptions of such compounds, unless otherwise specified, are intended to encompass all geometric and optical isomers, including racemates.

Some compounds described herein can exhibit tautomerism. The structural drawings herein typically represent only one of the possible tautomeric forms of such compounds. It will be understood that the structural drawings are intended to encompass all tautomeric forms of such compounds.

The term “pharmaceutically acceptable salts” refers those salts of the compounds of Formula (I) which retain the biological activity of the free compounds and which can be administered as a pharmaceutical to humans and/or animals. The desired salt of a basic functional group of a compound may be prepared by treating the compound with an acid. Some examples of suitable inorganic acids include, but are not limited to, hydrochloric acid, hydrobromic acid, sulfuric acid, nitric acid, and phosphoric acid. Some examples of suitable organic acids include, but are not limited to, formic acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, sulfonic acids, and salicylic acid. The desired salt of an acidic functional group of a compound can be prepared by treating the compound with a base. Some examples of suitable inorganic salts of acid compounds include, but are not limited to, alkali metal and alkaline earth salts, such as sodium salts, potassium salts, magnesium salts, and calcium salts; ammonium salts; and aluminum salts. Some examples of suitable of organic salts of acid compounds include, but are not limited to, procaine, dibenzylamine, N-ethylpiperidine, N,N′-dibenzylethylenediamine, and triethylamine salts.

Compounds of Formula (I) may contain the stated atoms in any of their isotopic forms. In this respect, embodiments of the invention that may be mentioned include those in which: (a) the compound of Formula (I) is not isotopically enriched or labelled with respect to any atoms of the compound; and (b) the compound of Formula (I) is isotopically enriched or labelled with respect to one or more atoms of the compound.

The use of “〰” in formulas herein denotes the point of attachment between different groups.

Illustrative compounds of Formula (I), or a pharmaceutically acceptable salt thereof, include:
5-amino-8-(2,6-dimethyl-4-pyridyl)-7-phenyl-2-(2-pyrazol-1-ylethyl)-[1,2,4]triazolo[4,3-c]pyrimidin-3-one;
5-amino-8-(2,6-dimethyl-4-pyridyl)-2-[2-(1-methylimidazol-2-yl)ethyl]-7-phenyl-[1,2,4]triazolo[4,3-c]pyrimidin-3-one;
5-amino-8-(2,6-dimethyl-4-pyridyl)-2-[2-(1H-imidazol-2-yl)ethyl]-7-phenyl-[1,2,4]triazolo[4,3-c]pyrimidin-3-one;
5-amino-8-(2,6-dimethyl-4-pyridyl)-7-phenyl-2-[2-(1H-tetrazol-5-yl)ethyl]-[1,2,4]triazolo[4,3-c]pyrimidin-3-one;
5-amino-8-(2,6-dimethyl-4-pyridyl)-2-[2-(1-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one;
5-amino-8-(2,6-dimethyl-4-pyridyl)-7-phenyl-2-[1-(2-thienyl)ethyl]-[1,2,4]triazolo[4,3-c]pyrimidin-3-one;
5-amino-8-(2,6-dimethyl-4-pyridyl)-2-(oxazol-2-ylmethyl)-7-phenyl-[1,2,4]triazolo[4,3-c]pyrimidin-3-one;
5-amino-8-(2,6-dimethyl-4-pyridyl)-2-(oxazol-4-ylmethyl)-7-phenyl-[1,2,4]triazolo[4,3-c]pyrimidin-3-one;
5-amino-8-(2,6-dimethyl-4-pyridyl)-2-[(1-methylimidazol-2-yl)methyl]-7-phenyl-[1,2,4]triazolo[4,3-c]pyrimidin-3-one;
5-amino-8-(2,6-dimethyl-4-pyridyl)-2-[(2-methylpyrazol-3-yl)methyl]-7-phenyl-[1,2,4]triazolo[4,3-c]pyrimidin-3-one;
5-amino-8-(2,6-dimethyl-4-pyridyl)-2-(isoxazol-3-ylmethyl)-7-phenyl-[1,2,4]triazolo[4,3-c]pyrimidin-3-one;
5-amino-8-(2,6-dimethyl-4-pyridyl)-2-[(5-methylisoxazol-3-yl)methyl]-7-phenyl-[1,2,4]triazolo[4,3-c]pyrimidin-3-one;
5-amino-8-(2,6-dimethyl-4-pyridyl)-2-[(2-methyloxazol-4-yl)methyl]-7-phenyl-[1,2,4]triazolo[4,3-c]pyrimidin-3-one;
5-amino-8-(2,6-dimethyl-4-pyridyl)-2-[(1-methylpyrazol-3-yl)methyl]-7-phenyl-[1,2,4]triazolo[4,3-c]pyrimidin-3-one;
5-amino-8-(2,6-dimethyl-4-pyridyl)-2-[(5-methyl-1,3,4-oxadiazol-2-yl)methyl]-7-phenyl-[1,2,4]triazolo[4,3-c]pyrimidin-3-one;
5-amino-8-(2,6-dimethyl-4-pyridyl)-2-[(3-methylimidazol-4-yl)methyl]-7-phenyl-[1,2,4]triazolo[4,3-c]pyrimidin-3-one;
5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one;
5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one;
5-amino-8-(2,6-dimethyl-4-pyridyl)-2-[(3-methyl-1,2,4-oxadiazol-5-yl)methyl]-7-phenyl-[1,2,4]triazolo[4,3-c]pyrimidin-3-one;
5-amino-8-(2,6-dimethyl-4-pyridyl)-2-[(5-methyloxazol-4-yl)methyl]-7-phenyl-[1,2,4]triazolo[4,3-c]pyrimidin-3-one;
5-amino-8-(2,6-dimethyl-4-pyridyl)-2-(1H-imidazol-5-ylmethyl)-7-phenyl-[1,2,4]triazolo[4,3-c]pyrimidin-3-one;
5-amino-8-(2,6-dimethyl-4-pyridyl)-2-(1H-imidazol-2-ylmethyl)-7-phenyl-[1,2,4]triazolo[4,3-c]pyrimidin-3-one;
5-amino-8-(2,6-dimethyl-4-pyridyl)-7-phenyl-2-(1H-pyrazol-5-ylmethyl)-[1,2,4]triazolo[4,3-c]pyrimidin-3-one;
5-amino-8-(2,6-dimethyl-4-pyridyl)-7-phenyl-2-(2H-tetrazol-5-ylmethyl)-[1,2,4]triazolo[4,3-c]pyrimidin-3-one;
5-amino-8-(2,6-dimethyl-4-pyridyl)-2-(1,3,4-oxadiazol-2-ylmethyl)-7-phenyl-[1,2,4]triazolo[4,3-c]pyrimidin-3-one;
5-amino-8-(2,6-dimethyl-4-pyridyl)-2-[(4-methyl-1,2,4-triazol-3-yl)methyl]-7-phenyl-[1,2,4]triazolo[4,3-c]pyrimidin-3-one;
5-amino-8-(2,6-dimethyl-4-pyridyl)-2-[(5-methyl-1H-triazol-4-yl)methyl]-7-phenyl-[1,2,4]triazolo[4,3-c]pyrimidin-3-one;
5-amino-8-(2,6-dimethyl-4-pyridyl)-2-[(2-methyltriazol-4-yl)methyl]-7-phenyl-[1,2,4]triazolo[4,3-c]pyrimidin-3-one;
5-amino-8-(2,6-dimethyl-4-pyridyl)-7-(4-fluorophenyl)-2-[(5-methyloxazol-4-yl)methyl]-[1,2,4]triazolo[4,3-c]pyrimidin-3-one;
5-amino-2-[(2,5-dimethyloxazol-4-yl)methyl]-8-(2,6-dimethyl-4-pyridyl)-7-(4-fluorophenyl)-[1,2,4]triazolo[4,3-c]pyrimidin-3-one;
5-amino-2-[[1-benzyl-3-(3-methoxyphenyl)pyrazol-4-yl]methyl]-8-(2,6-dimethyl-4-pyridyl)-7-phenyl-[1,2,4]triazolo[4,3-c]pyrimidin-3-one;
5-amino-8-(2,6-dimethyl-1-oxido-pyridin-1-ium-4-yl)-2-[(5-methyloxazol-4-yl)methyl]-7-phenyl-[1,2,4]triazolo[4,3-c]pyrimidin-3-one;
5-amino-2-[(2,5-dimethyloxazol-4-yl)methyl]-8-(2,6-dimethyl-1-oxido-pyridin-1-ium-4-yl)-7-(4-fluorophenyl)-[1,2,4]triazolo[4,3-c]pyrimidin-3-one;
5-amino-8-(2,6-dimethyl-1-oxido-pyridin-1-ium-4-yl)-2-[(1-methylimidazol-2-yl)methyl]-7-phenyl-[1,2,4]triazolo[4,3-c]pyrimidin-3-one;
5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one;
5-amino-8-(2-methoxy-6-methyl-4-pyridyl)-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-2-ylmethyl)-7-phenyl-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-4-ylmethyl)-7-phenyl-[1,2,4]triazolo[4,3-c]pyrimidin-3-one;
5-amino-8-[2-(hydroxymethyl)-6-methyl-4-pyridyl]-2-[(5-methylisoxazol-3-yl)methyl]-7-phenyl-[1,2,4]triazolo[4,3-c]pyrimidin-3-one;
5-amino-2-[(3,5-dimethylimidazol-4-yl)methyl]-8-[2-(hydroxymethyl)-6-methyl-4-pyridyl]-7-phenyl-[1,2,4]triazolo[4,3-c]pyrimidin-3-one;
5-amino-8-[2-(hydroxymethyl)-6-methyl-4-pyridyl]-2-[(1-methylpyrazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-(oxazol-4-ylmethyl)-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methyl-4-pyridyl]-2-[(1-methylimidazol-2-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(1-methylpyrazol-3-yl)methyl]-[1,2,4]triazolo[4,3-c]pyrimidin-3-one;
5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-7-(4-fluorophenyl)-2-[(1-methylimidazol-2-yl)methyl]-[1,2,4]triazolo[4,3-c]pyrimidin-3-one;
5-amino-8-[2-chloro-6-(hydroxymethyl)-4-pyridyl]-7-(4-fluorophenyl)-2-[(5-methyloxazol-4-yl)methyl]-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methoxy-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methoxy-4-pyridyl]-2-[(1-methylimidazol-2-yl)methyl]-[1,2,4]triazolo[4,3-c]pyrimidin-3-one;
5-amino-8-[2-(hydroxymethyl)-6-(trifluoromethyl)-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-(trifluoromethyl)-4-pyridyl]-2-[(1-methylimidazol-2-yl)methyl]-7-phenyl-[1,2,4]triazolo[4,3-c]pyrimidin-3-one;
tert-butyl 3-[5-amino-8-(2,6-dimethyl-4-pyridyl)-3-oxo-7-phenyl-[1,2,4]triazolo[4,3-c]pyrimidin-2-yl]pyrrolidine-1-carboxylate;
5-amino-8-(2,6-dimethyl-4-pyridyl)-7-phenyl-2-pyrrolidin-3-yl-[1,2,4]triazolo[4,3-c]pyrimidin-3-one;
5-amino-8-(2,6-dimethyl-4-pyridyl)-2-(1-methylpyrrolidin-3-yl)-7-phenyl-[1,2,4]triazolo[4,3-c]pyrimidin-3-one;
tert-butyl 2-[[5-amino-8-(2,6-dimethyl-4-pyridyl)-3-oxo-7-phenyl-[1,2,4]triazolo[4,3-c]pyrimidin-2-yl]methyl]pyrrolidine-1-carboxylate;
5-amino-8-(2,6-dimethyl-4-pyridyl)-7-phenyl-2-(pyrrolidin-2-ylmethyl)-[1,2,4]triazolo[4,3-c]pyrimidin-3-one;
5-amino-8-(2,6-dimethyl-4-pyridyl)-2-[(1-methylpyrrolidin-2-yl)methyl]-7-phenyl-[1,2,4]triazolo[4,3-c]pyrimidin-3-one;
5-amino-8-(2,6-dimethyl-4-pyridyl)-2-[[(2R)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-(2-methoxyethyl)pyrrolidin-2-yl]methyl]-7-phenyl-[1,2,4]triazolo[4,3-c]pyrimidin-3-one;
5-amino-8-(2,6-dimethyl-4-pyridyl)-2-[[(2R)-1-(2-methoxyethyl)pyrrolidin-2-yl]methyl]-7-phenyl-[1,2,4]triazolo[4,3-c]pyrimidin-3-one;
5-amino-2-[[(2S)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2S)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one;
5-amino-8-(2,6-dimethyl-4-pyridyl)-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[[(2S)-tetrahydrofuran-2-yl]methyl]-[1,2,4]triazolo[4,3-c]pyrimidin-3-one;
5-amino-8-(2,6-dimethyl-4-pyridyl)-2-[[(2R,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-7-phenyl-2-(2-pyrrolidin-1-ylethyl)-[1,2,4]triazolo[4,3-c]pyrimidin-3-one;
5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2S)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methyl-4-pyridyl]-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-phenyl-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(4-fluorophenyl)-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methoxy-4-pyridyl]-2-[[(2R)-tetrahydrofuran-2-yl]methyl]-[1,2,4]triazolo[4,3-c]pyrimidin-3-one;
5-amino-8-[2-(hydroxymethyl)-6-(trifluoromethyl)-4-pyridyl]-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one;
5-amino-8-(2,6-dimethyl-4-pyridyl)-7-phenyl-2-[2-[[1-(pyridine-3-carbonyl)pyrrolidin-3-yl]amino]ethyl]-[1,2,4]triazolo[4,3-c]pyrimidin-3-one;
5-amino-8-(2,6-dimethyl-4-pyridyl)-2-[2-[methyl(1H-pyrazol-4-yl)amino]ethyl]-7-phenyl-[1,2,4]triazolo[4,3-c]pyrimidin-3-one;
5-amino-2-[[1-[[2-(aminomethyl)phenyl]methyl]pyrrolidin-2-yl]methyl]-8-(2,6-dimethyl-4-pyridyl)-7-phenyl-[1,2,4]triazolo[4,3-c]pyrimidin-3-one; and
5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one.

Compounds of Formula (I) can be adenosine receptor antagonists, i.e. antagonists of one or more of A1R, A2aR, A2bR, and A3R. The term “adenosine receptor antagonist” refers to a compound, e.g., a compound of Formula (I) that binds to the adenosine receptor and antagonizes its activity.

In some cases, the compound of Formula (I) is a selective adenosine receptor antagonist. The term “selective” refers the property of a compound of Formula (I) that is an adenosine receptor antagonist but is substantially inactive at other biological targets. The term “substantially inactive” as used herein describes a compound that (i) has significantly weaker affinity for a given receptor as compared to its affinity for the adenosine receptor; (ii) does not show substantial agonist or antagonist activity at a given receptor; or both (i) and (ii).

The term “selective adenosine receptor antagonist” refers to a compound that shows binding affinity for one or more adenosine receptor subtypes that is at least 100 times greater, at least 1,000 times greater, or at least 10,000 times greater than its affinity for a given receptor. In other words, the ratio of binding Ki values (given receptor:adenosine receptor) can be at least 100, at least 1,000, or at least 10,000.

In particular, a selective adenosine receptor antagonist can be substantially inactive toward other G-protein coupled receptors, such as the cannabinoid receptors, referred to as CB-1 and CB-2.

A compound of Formula (I) can have a binding affinity Ki for A2aR of, e.g., 100 nM or less, 10 nM or less, or 1 nM or less.

A compound of Formula (I) can have a binding affinity Ki for A2bR of, e.g., 100 nM or less, 10 nM or less, or 1 nM or less.

A compound of Formula (I) can have a binding affinity Ki for CB-1 of, e.g., 1,000 nM or greater, 10,000 nM or greater, 13,000 nM or greater.

A compound of Formula (I) can be a selective adenosine receptor antagonist with respect to CB-1.

A compound of Formula (I) can be active as an adenosine receptor antagonist but substantially inactive at CB-1.

The compounds of Formula (I) can also be selective between the different subtypes of adenosine receptor. In some embodiments, the compounds of Formula (I) are A2aR-selective; A2bR-selective; or dual A2aR/A2bR-selective.

An A2aR-selective compound shows a binding affinity for A2aR that is at least 100 times stronger, at least 1,000 times stronger, or at least 10,000 times stronger than its binding affinity for each of A1R, A2bR, and A3R.

An A2bR-selective compound that is at least 100 times stronger, at least 1,000 times stronger, or at least 10,000 times stronger than its binding affinity for each of A1R, A2aR, and A3R.

A dual A2aR/A2bR-selective compound shows a binding affinity for A2aR that is at least 100 times stronger, at least 1,000 times stronger, or at least 10,000 times stronger than its binding affinity for each of A1R and A3R. A dual A2aR/A2bR-selective also shows a binding affinity for A2bR that is at least 100 times stronger, at least 1,000 times stronger, or at least 10,000 times stronger than its binding affinity for each of A1R and A3R. In addition, for a dual A2aR/A2bR-selective compound, the ratio of binding affinity for A2aR to binding affinity for A2bR is less than 100.

In one embodiment, there is provided a pharmaceutical composition which includes a compound of Formula (I), or a pharmaceutically acceptable salt thereof, and a pharmaceutically acceptable carrier, diluent, or excipient.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

Suitable pharmaceutically acceptable excipients for a tablet formulation include, for example, inert diluents such as lactose, sodium carbonate, calcium phosphate or calcium carbonate; granulating and disintegrating agents such as corn starch or algenic acid; binding agents such as starch; lubricating agents such as magnesium stearate, stearic acid or talc; preservative agents such as ethyl or propyl p-hydroxybenzoate; and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Compounds of Formula (I) are useful in the treatment of diseases or conditions mediated by the adenosine receptor. In one embodiment, there is provided a compound of Formula (I) or a pharmaceutically acceptable salt thereof, for use in the treatment of diseases or conditions mediated by the adenosine receptor. In some embodiments the disease or condition is mediated by A2aR; in other embodiments, by A2bR; in still other embodiments, by both A2aR and A2bR.

Some examples of disease or conditions mediated by the adenosine receptor include cancer, such as lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor; movement disorders, such as Parkinson’s disease and Huntington’s disease; and attention disorders, such as attention deficit disorder and attention deficit-hyperactivity disorder. Other diseases and conditions mediated by the adenosine receptor are known.

In one embodiment, there is provided a compound of Formula (I), or a pharmaceutically acceptable salt thereof, for use in the treatment of a disease or condition mediated by the adenosine receptor.

In one embodiment, there is provided a compound of Formula (I), or a pharmaceutically acceptable salt thereof, for use in the treatment of cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor.

In one embodiment, there is provided a compound of Formula (I), or a pharmaceutically acceptable salt thereof, for use in the treatment of a disease or condition mediated by the adenosine receptor, wherein the compound is a selective adenosine receptor antagonist with respect to CB-1.

In one embodiment, there is provided a compound of Formula (I), or a pharmaceutically acceptable salt thereof, for use in the treatment of cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor, wherein the compound is a selective adenosine receptor antagonist with respect to CB-1.

In one embodiment, there is provided a method of treating a disease or condition mediated by the adenosine receptor, which includes administering an effective amount of a compound of Formula (I), or a pharmaceutically acceptable salt thereof, to a subject in need of such treatment.

In one embodiment, there is provided a method of treating cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor), which includes administering an effective amount of a compound of Formula (I), or a pharmaceutically acceptable salt thereof, to a subject in need of such treatment.

In one embodiment, there is provided a method of treating a disease or condition mediated by the adenosine receptor, which includes administering an effective amount of a compound of Formula (I), or a pharmaceutically acceptable salt thereof, to a subject in need of such treatment, wherein the compound is a selective adenosine receptor antagonist with respect to CB-1.

In one embodiment, there is provided a method of treating cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor), which includes administering an effective amount of a compound of Formula (I), or a pharmaceutically acceptable salt thereof, to a subject in need of such treatment, wherein the compound is a selective adenosine receptor antagonist with respect to CB-1.

In one embodiment, there is provided a compound of Formula (I), or a pharmaceutically acceptable salt thereof, for use in the manufacture of a medicament for use in the treatment of a disease or condition mediated by the adenosine receptor.

In one embodiment, there is provided a compound of Formula (I), or a pharmaceutically acceptable salt thereof, for use in the manufacture of a medicament for use in the treatment of cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor).

In one embodiment, there is provided a compound of Formula (I), or a pharmaceutically acceptable salt thereof, for use in the manufacture of a medicament for use in the treatment of a disease or condition mediated by the adenosine receptor, wherein the compound is a selective adenosine receptor antagonist with respect to CB-1.

In one embodiment, there is provided a compound of Formula (I), or a pharmaceutically acceptable salt thereof, for use in the manufacture of a medicament for use in the treatment of cancer (including lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor, wherein the compound is a selective adenosine receptor antagonist with respect to CB-1.

Compounds of Formula (I) can be prepared according to the following general schemes.

Schemes 1a and 1b illustrate the preparation of intermediate 6-substituted-4-hydrazino-2-aminopyrimidine compounds of Formula (IV).

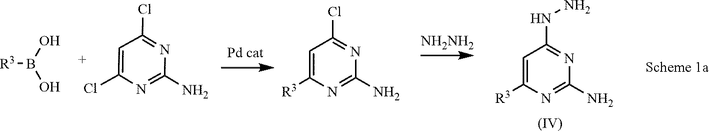

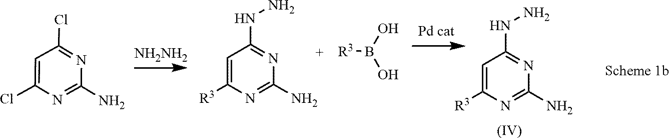

Scheme 2 illustrates the conversion of compounds of Formula (IV) into the intermediate 7-substituted-5-amino-8-bromo-[1,2,4]triazolo[4,3-c]pyrimidin-3-one compounds of Formula (V). Briefly, the compound of Formula (IV) is treated with triphosgene to effect closure of the triazolone ring, followed by bromination with (CH₃₎₃PhN⁺Br₃⁻.

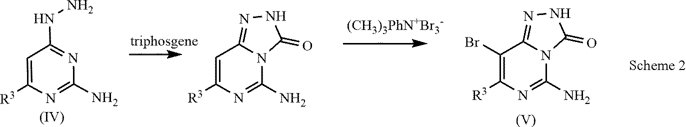

Scheme 3a illustrates the conversion of compounds of Formula (V) into compound of Formula (I). The alkylation of the compound of Formula (V) with R⁴ can be carried out using a variety of methods, for example, Mitsonobu reaction; alcohol mesylation followed by an alkylation reaction; alcohol tosylation followed by an alkylation reaction; or alcohol chlorination followed by an alkylation reaction.

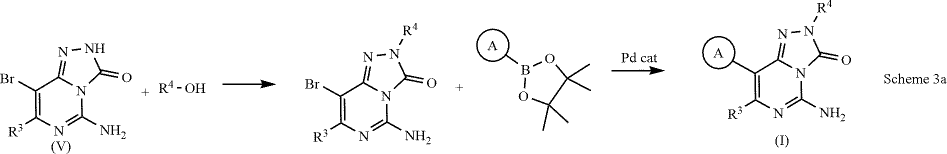

Alternatively, a compound such as R⁴—Br may be used in a direct alkylation of the compound of Formula (V).

Optionally, R⁴ can be further modified after alkylation of the compound of Formula (V).

Scheme 3b illustrates an alternate route for the conversion of compounds of Formula (V) into compounds of Formula (I). In Scheme 3b, [Pg] represents a suitable reagent for installing the protecting group denoted Pg. The alkylation of the compound of Formula (Va) with R⁴ can be carried out using a variety of methods, for example, Mitsonobu reaction; alcohol mesylation followed by an alkylation reaction; alcohol tosylation followed by an alkylation reaction; alcohol chlorination followed by an alkylation reaction.

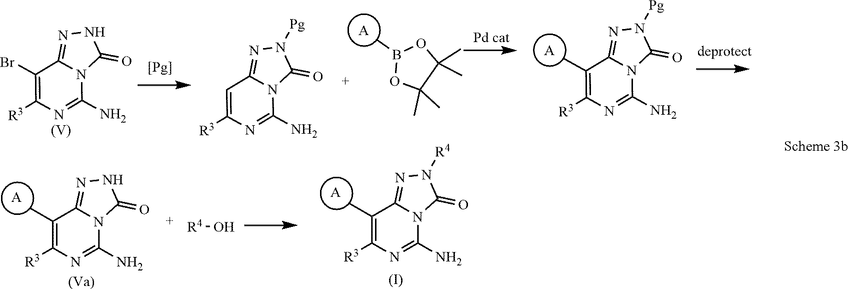

Alternatively, a compound such as R⁴—Br may be used in a direct alkylation of the compound of Formula (Va).

Optionally, R⁴ can be further modified after alkylation of the compound of Formula (V).

Optionally, a compound of Formula (I) can be further modified, for example, to form a different compound of Formula (I).

### EXAMPLES

### General Techniques

### LCMS Method A

Instrument: Agilent Technologies 1200 Series, Agilent LC/MSD SL, Column: Waters XBridge C8 3.5 µm, 4.6 x 50 mm. Gradient [time (min)/solvent B(%)]:0.0/5,8.0/100,8.1/100,8.5/5,10.0/5. (Solvent A=1 mL of TFA in 1000 mL of Milli-Q Water; Solvent B=1 mL of TFA in 1000 mL of MeCN); Injection volume 1 µL (may vary); UV detection 220 to 400 nm; Column temperature 25° C.; 2.0 mL/min.

For UV inactive compounds an ELSD detector (Polymer Laboratories PL-ELS 2100 ICE) is connected with the above instrument.

### LCMS Method B

Instrument: Agilent Technologies 1200 Series, Agilent LC/MSD SL, Column: Atlantis dC18 5 µm, 4.6×50 mm. Gradient [time (min)/solvent B (%)]:0.0/10, 2.5/95, 4.5/95, 4.6/10, 6.0/10. (Solvent A=1 mL of TFA in 1000 mL of Milli-Q Water; Solvent B=1 mL of TFA in 1000 mL of MeCN); Injection volume 1 µL (may vary); UV detection 210 to 400 nm; Column temperature 25° C.; 1.5 mL/min.

### LCMS Method C

Instrument: Agilent Technologies 1200 Series, Agilent 6130 Quadrupole LC/MS, Column: Zorbax C18 5 µm, 4.6 × 50 mm. Gradient [time (min)/solvent B (%)]:0.0/10, 2.5/95, 4.5/95, 4.6/10, 6.0/10. (Solvent A=1 mL of Formic Acid in 1000 mL of Milli-Q Water; Solvent B= MeCN); Injection volume 1 µL (may vary); UV detection 210 to 400 nm; column temperature 25° C.; 1.5 mL/min.

### LCMS Method D

Instrument: Agilent Technologies 1200 Series, Agilent 6130 Quadrupole LC/MS, Column: Zorbax C18 5 µm, 4.6 × 50 mm. Gradient [time (min)/solvent B (%)]:0.0/10, 4.0/95, 5.0/95, 5.5/10, 7.0/10. (Solvent A=770.08 mg of Ammonium acetate in 1000 mL of Milli-Q Water; Solvent B= MeCN); Injection volume 1 µL (may vary); UV detection 210 to 400 nm; column temperature 25° C.; 1.2 mL/min.

### LCMS Method E

Instrument: Agilent Technologies 1200 Series, Agilent 6130 Quadrupole LC/MS, Column: XBridge C8 3.5 µm, 4.6 × 50 mm. Gradient [time (min)/solvent B (%)]:0.0/5, 8.0/100, 8.1/100, 8.5/5, 10.0/5. (Solvent A=790.06 mg of Ammonium bicarbonate is added to 1000 mL of Milli-Q Water; Solvent B= MeCN); Injection volume 1 µL (may vary); UV detection 210 to 400 nm; column temperature 25° C.; 1.0 mL/min.

### LCMS Method F

Instrument: Agilent 1100 Series LC/MSD. Column: Zorbax SB-C18 1.8 µm 4.6×15 mm. Gradient [time (min)/solvent A(%)]:0.0/100; 0.01/100; 1.5/0; 1.8/0; 1.81/100. (Solvent A = H₂O; Solvent B = MeCN, both modified with 0.1% formic acid). Injection volume 1 µL (may vary). UV detection 215 nm. Column temperature 60° C.

### LCMS Method G

Instrument: Waters Acquity UPLC with Waters ELSD and Waters SQD mass spectrometer, Column: Waters Acquity HSS T3 1.8 µm, 2.1 × 30 mm. Gradient [time (min)/solvent B (%)]:0.0/2, 1.5/98, 1.9/98, 1.95/2, 2.0/2. (Solvent A=1 mL of Formic Acid in 1000 mL of HPLC-grade Water; Solvent B= 1 mL of Formic Acid in 1000 mL of MeCN); Injection volume 1 µL; UV detection 210 to 400 nm; column temperature 25° C.; 1 mL/min.

### Prep-HPLC Method A

Instrument: Agilent Technologies 1260 Infinity II Series LC. Solvent: A- 0.1% TFA in H₂O, B-MeOH, Column: YMC Actus Triart C18 (30 mm × 250 mm) 5 µm. Gradient [time (min)/solvent B (%)]:0.0/10, 20/95, 23/95, 24/10, 26/10.

### Prep-HPLC Method B

Instrument: Agilent Technologies 1260 Infinity II Series LC. Solvent: A- 0.1% HCOOH in H₂O, B- MeCN, Column: YMC Actus Triart C8 (20 mm × 250 mm) 5 µm. Gradient [time (min)/solvent B (%)]:0.0/10, 20/95, 23/95, 24/10, 26/10.

### Prep-HPLC Method C

Instrument: Agilent Technologies 1260 Infinity II Series LC. Solvent: A- 10 mM NH₄HCO₃ in H₂O, B-MeOH or MeCN, Column: XBridge C8 (19 mm × 150 mm), 5 um or YMC Actus Triart C18 (30 mm × 250 mm) 5 µm. Gradient [time (min)/solvent B (%)]:0.0/10, 15/95, 18/95, 19/10, 21/10.

### Prep-HPLC Method D

Instrument: Agilent Technologies 1260 Infinity II Series LC. Mobile Phase: HEXANE B: IPA (60:40), Column: YMC Silica (19x150) mm, 5 µm, Flow: 15 mL/min. Note: Gradient may vary from sample to sample based on sample separation and Polarity.

### Prep-HPLC Method E

Instrument: Agilent Technologies 1260 Infinity II Series LC. Solvent: A - H₂O, B-MeOH or MeCN. Column: Waters Sunfire C18 OBD Prep Column, 100 Å, 5 µm, 19 mm×100 mm. Gradient [time (min)/solvent B (%)]:0.0/10, 20/95, 23/95, 24/10, 26/10.

### SFC Method A

Instrument: Thar Multigram III Preparative SFC. Solvent: A- CO₂, B-2 mL of NH₄OH in 1000 mL of MeOH, Column: CHIRALPAK IB 5 µm, 21 × 250 mm. Isocratic 15%; Outlet Pressure 100 bar; UV detection 220 nm; Column temperature 35° C.; 70.0 mL/min.

### Synthetic Routes for Intermediates

Synthetic routes 1 to 8, used to prepare Intermediates used in the synthesis of compounds of Formula (I), are described below. The details of synthetic routes 1 to 8 are illustrative of the techniques used in the preparation of other Intermediates as detailed in Table 1 below.

### Synthetic Route 1: Procedure For The Preparation of Intermediate 1

Intermediate 1: 5-amino-8-bromo-7-phenyl-[1,2,4]triazolo[4,3-c]pyrimidin-3(2H)-one

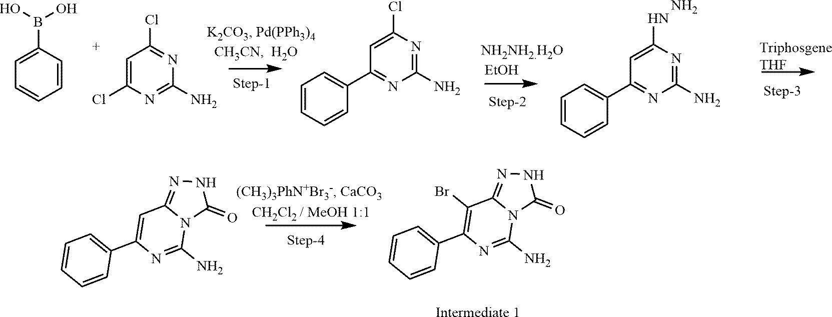

Step 1: This reaction was performed as 2 × 250 g batches. To a degassed suspension of phenyl boronic acid (250 g, 2.05 mol), 4,6-dichloro-2-aminopyrimidine (672 g, 4.10 mol) and K₂CO₃ (848 g, 6.15 mol) in CH₃CN (15 L) and H₂O (2 L) at room temperature was added Pd(PPh₃)₄ (118 g, 0.10 mol) and the resultant reaction mixture was heated to 90° C. for 6 h. The reaction mixture was concentrated under reduced pressure. The residue obtained was vigorously stirred with H₂O (4 L) and DCM (10 L), undissolved solids were filtered-off through a Buchner funnel and rinsed with DCM (3 L). The filtrate was taken in a separating funnel, the organic layer was separated, dried over anhydrous Na₂SO₄ and concentrated under reduced pressure. The crude product was purified by flash chromatography using 230-400 silica mesh and was eluted with 0-15% EtOAc in Pet-Ether to afford 4-chloro-6-phenylpyrimidin-2-amine (350 g, 41%) as an off-white solid.

LCMS (Method A): m/z 206 (M+H)⁺ (ES⁺), at 2.53 min, UV active.

¹H NMR: (400 MHz, DMSO-d6) δ: 8.05 - 8.03 (m, 2H), 7.52 - 7.47 (m, 3 H), 7.21 (s, 1H). Exchangeable —NH2 protons were not observed.

Step 2: To a stirred suspension of 4-chloro-6-phenylpyrimidin-2-amine (350 g, 1.70 mol) in EtOH (4.0 L), hydrazine hydrate (255 g, 5.1 mol) was added and the mixture was heated to 90° C. for 15 h. The reaction was concentrated under reduced pressure. The residue obtained was triturated with diethyl ether (1 L) and 10% sodium bicarbonate solution (1 L). The solid obtained was collected by filtration through a Buchner funnel, rinsed with Diethyl ether (200 mL) and dried under vacuum to afford 4-hydrazinyl-6-phenylpyrimidin-2-amine (250 g, 73%) as an off-white solid.

LCMS (Method C): m/z 202 (M+H)⁺ (ES⁺), at 0.69 min, UV active.

¹H NMR: (400 MHz, DMSO-d6) δ: 7.94 - 7.91 (m, 2H), 7.84 (s, 1H), 7.48 - 7.42 (m, 3H), 6.47 (s, 1H), 6.00 (s, 2H), 4.25 (s, 2H).

Step 3: To a solution of 4-hydrazinyl-6-phenylpyrimidin-2-amine (250 g, 1.24 mol) in dry THF (3.0 L) under N₂, cooled to -30° C. was added triphosgene (735 g, 2.48 mol) portion wise and the mixture was stirred at same temperature for 45 min. The reaction was quenched cautiously into ice cold water (10 L) with vigorous stirring. After the effervescence stopped, the reaction mass was concentrated under reduced pressure. The resulting solid was collected by filtration through a Buchner funnel, rinsed with water (1 L) and dried under vacuum to afford 5-amino-7-phenyl-[1,2,4]triazolo[4,3-c]pyrimidin-3(2H)-one (200 g, 70%) as a yellow solid.

LCMS (Method C): m/z 228 (M+H)⁺ (ES⁺), at 1.64 min, UV active.

¹H NMR: (400 MHz, DMSO-d6) δ: 12.46 (s, 1H), 8.05 - 7.98 (m, 3H), 7.65 (s, 1H), 7.50 - 7.44 (m, 3H), 6.93 (s, 1H).

Step 4: To a suspension of 5-amino-7-phenyl-[1,2,4]triazolo[4,3-c]pyrimidin-3(2H)-one (200 g, 0.88 mol) in DCM/MeOH 1:1 (2 L) under N₂ atmosphere, CaCO₃ (88 g, 0.88 mol) followed by (CH₃)₃PhN⁺ Br₃⁻ (331 g, 0.88 mol) were added and the mixture was stirred at room temperature for 1 h. The reaction mixture was filtered through a Buchner funnel, rinsed with small portions of MeOH/DCM (1:1) and dried under vacuum to afford Intermediate 1, 5-amino-8-bromo-7-phenyl-[1,2,4]triazolo[4,3-c]pyrimidin-3(2H)-one (160 g, 59%) as a light brown solid.

LCMS (Method C): m/z 306 (M+H)⁺ (ES⁺), at 1.78 min, UV active.

¹H NMR: (400 MHz, DMSO-d6) δ: 12.57 (s, 1H), 7.62 - 7.60 (m, 2H), 7.45 - 7.41 (m, 3H). Exchangeable —NH₂ protons were not observed.

### Synthetic Route 2: Procedure For The Preparation of Intermediate 6

Intermediate 6: 5-amino-8-(2,6-dimethylpyridin-4-yl)-7-phenyl-[1,2,4]triazolo[4,3-c]pyrimidin-3(2H)-one

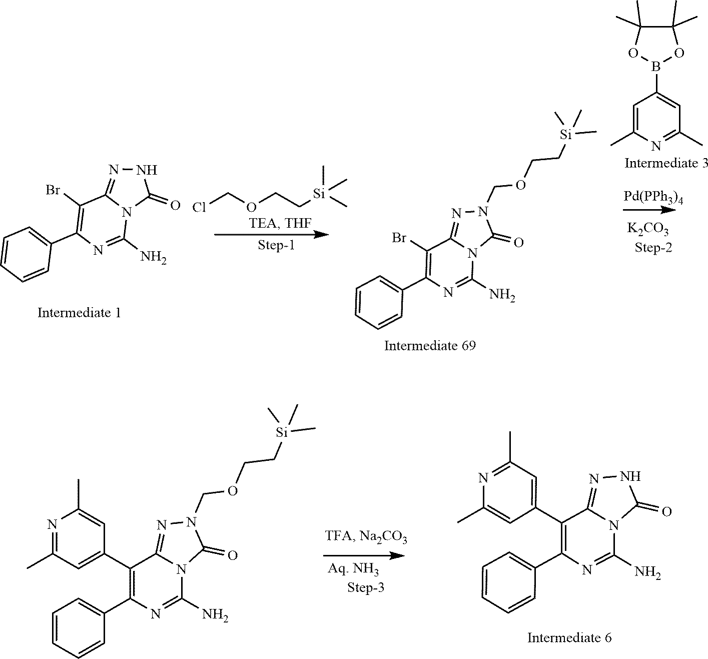

Step 1: To a suspension of 5-amino-8-bromo-7-phenyl-[1,2,4]triazolo[4,3-c]pyrimidin-3(2H)-one (16.2 g, 53 mmol) in THF (200 mL) at 0° C. was added TEA (19 mL, 136.3 mmol) followed by the dropwise addition of (2-(chloromethoxy)ethyl)trimethylsilane (11.3 g, 67.8 mmol). The reaction was stirred at 0° C. for 1 h then partitioned between EtOAc (250 mL) and water (200 mL). The organic layer was separated, dried over anhydrous Na₂SO₄ and concentrated under reduced pressure. The crude product was purified by Biotage-Isolera using 100 g silica snap and eluted with gradient 0-30% EtOAc in Hexane to afford Intermediate 69, 5-amino-8-bromo-7-phenyl-2-((2-(trimethylsilyl)ethoxy)methyl)-[1,2,4]triazolo[4,3-c]pyrimidin-3(2H)-one (12 g, 52%) as an off-white solid.

LCMS (Method B): m/z 436 (M+H)⁺ (ES⁺), at 3.25 min, UV active.

¹H NMR: (400 MHz, DMSO-d6) δ: 8.56 (s, 2H), 7.62 (d, J = 7.1 Hz, 2H), 7.45 (d, J = 6.6 Hz, 3H), 5.18 (s, 2H), 3.66 (t, J = 8.2 Hz, 2H), 0.91 (t, J = 8.2 Hz, 2H), 0.04 (s, 9H).

Step 2: To a degassed suspension of 5-amino-8-bromo-7-phenyl-2-((2-(trimethylsilyl)ethoxy)methyl)-[1,2,4]triazolo[4,3-c]pyrimidin-3(2H)-one (11 g, 25 mmol), 2,6-dimethyl-4-(4,4,5,5-tetramethyl-1,3,2-dioxaborolan-2-yl)pyridine (6.5 g, 28 mmol) and K₂CO₃ (8.6 g, 62.5 mmol) in 1,4-Dioxane (150 mL) and water (30 mL) at room temperature was added Pd(PPh₃)₄ (1.44 g, 1.25 mmol) and the reaction mixture was heated at 120° C. for 5 h. The reaction mixture was partitioned between EtOAc (300 mL) and water (200 mL). The organic layer was separated, dried over anhydrous Na₂SO₄ and concentrated under reduced pressure. The crude product was purified by Biotage-Isolera using 100 g silica snap and eluted with gradient 0-80% EtOAc in Hexane to afford 5-amino-8-(2,6-dimethylpyridin-4-yl)-7-phenyl-2-((2-(trimethylsilyl)ethoxy)methyl)-[1,2,4]triazolo[4,3-c]pyrimidin-3(2H)-one (7.5 g, 64%) as a yellow solid.

LCMS (Method B): m/z 462 (M+H)⁺ (ES⁺), at 2.55 min, UV active.

¹H NMR: (400 MHz, DMSO-d6) δ: 7.30 - 7.26 (m, 5H), 6.82 (s, 2H), 5.13 (s, 2H), 3.63 (t, J = 7.4 Hz, 2H), 2.29 (s, 6H), 0.88 (t, J = 7.4 Hz, 2H), 0.06 (s, 9H). Exchangeable —NH₂ protons were not observed.

Step 3: 5-amino-8-(2,6-dimethylpyridin-4-yl)-7-phenyl-2-((2-(trimethylsilyl)ethoxy)methyl)-[1,2,4]triazolo[4,3-c]pyrimidin-3(2H)-one (7 g, 15 mmol) was dissolved in TFA (40 mL) and stirred at room temperature for 30 min. The reaction mixture was concentrated under reduced pressure and dried under hi-vacuum. The residue obtained was taken in EtOH (30 mL) and cautiously added Aq. NH₄OH (50 mL) and the reaction mixture was heated at 60° C. for 2 h. The solid was collected by filtration through a Buchner funnel, washed with water (10 mL) and EtOH (10 mol) and dried under vacuum to afford Intermediate 6, 5-amino-8-(2,6-dimethylpyridin-4-yl)-7-phenyl-[1,2,4]triazolo[4,3-c]pyrimidin-3(2H)-one (4.5 g, 89%) as a yellow solid.

LCMS (Method A): m/z 333 (M+H)⁺ (ES⁺), at 1.98 min, UV active.

¹H NMR: (400 MHz, DMSO-d6) δ: 12.25 (s, 1H), 8.14 (s, 2H), 7.29 - 7.25 (m, 5H), 6.82 (s, 2H), 2.29 (s, 6H).

### Synthetic Route 3: Procedure For The Preparation of Intermediate 26

Intermediate 26: 5-amino-8-bromo-7-(4-fluorophenyl)-[1,2,4]triazolo[4,3-c]pyrimidin-3(2H)-one

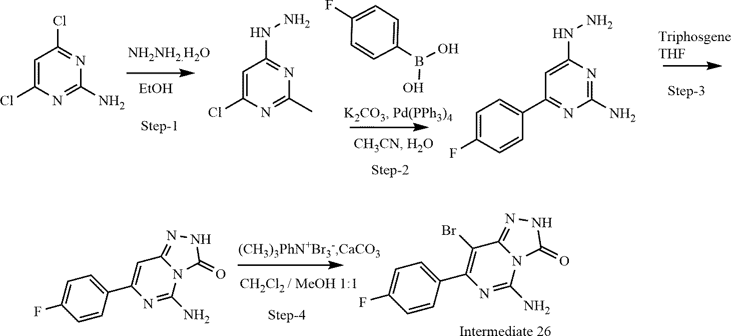

Step 1: To a stirred suspension of 4,6-dichloropyrimidin-2-amine (400 g, 2.43 mol) in EtOH (5 L), was added hydrazine hydrate (365 g, 7.31 mol) and the mixture was heated to 90° C. for 15 h. The reaction mass was concentrated under reduced pressure. The residue obtained was triturated with diethyl ether (1 L) and 10% sodium bicarbonate solution (1 L). The solid obtained was collected by filtration through a Buchner funnel, rinsed with Diethyl ether (200 mL) and dried under vacuum to afford 4-chloro-6-hydrazineylpyrimidin-2-amine (300 g, 77%) as an off-white solid.

LCMS (Method C): m/z 160 (M+H)⁺ (ES⁺), at 0.37 min, UV active.

¹H NMR: (400 MHz, DMSO-d6) δ: 8.10 (s, 1H), 6.36 (s, 2H), 5.97 (s, 1H), 4.26 (s, 2H).

Step 2: To a degassed suspension of 4-chloro-6-hydrazineylpyrimidin-2-amine (300 g, 1.87 mol), 4-Fluorophenyl boronic acid (313 g, 2.24 mol), and K₂CO₃ (774 g, 5.61 mol) in 1,4-dioxane (6 L) and H₂O (1 L) at room temperature was added Pd(PPh₃)₄ (107 g, 0.093 mol) and the resultant reaction mixture was heated to 110° C. for 15 h. The reaction mixture was concentrated under reduced pressure to remove the 1,4-dioxane. The residue obtained was vigorously stirred with H₂O (4 L) to obtain a solid, which was filtered through Buchner funnel and rinsed with MeOH (1 L). The solid was dried under vacuum to afford 4-(4-fluorophenyl)-6-hydrazineylpyrimidin-2-amine (200 g, 49%) as a green solid.

LCMS (Method C): m/z 220 (M+H)⁺ (ES⁺), at 0.76 min, UV active.

¹H NMR: (400 MHz, DMSO-d6) δ: 8.00 - 7.96 (m, 2H), 7.854 (s,1H), 7.29 - 7.24 (m, 2H), 6.45 (s, 1H), 6.01 (s, 2H), 4.24 (s, 2H).

Step 3: To a solution of 4-(4-fluorophenyl)-6-hydrazineylpyrimidin-2-amine (200 g, 0.91 mol) in dry THF (3.0 L) under N₂, cooled to -30° C. was added triphosgene (538 g, 1.82 mol) portionwise and the mixture was stirred at same temperature for 1 h. The reaction was quenched cautiously into ice cold water (10 L) with vigorous stirring. After the effervescence stopped, the reaction mass was concentrated under reduced pressure. The resulting solid was collected by filtration through a Buchner funnel, rinsed with water (1 L) and dried under vacuum to afford 5-amino-7-(4-fluorophenyl)-[1,2,4]triazolo[4,3-c]pyrimidin-3(2H)-one (150 g, 67%) as yellow solid.

LCMS (Method C): m/z 246 (M+H)⁺ (ES⁺), at 1.77 min, UV active.

¹H NMR: (400 MHz, DMSO-d6) δ: 12.43 (s, 1H), 8.19 - 8.01 (m, 2H), 7.95 - 7.52 (m, 2H), 7.50 - 7.27 (m, 2H), 6.92 (s, 1H).

Step 4: This reaction was performed on 2 × 75 g batches. To a suspension of 5-amino-7-(4-fluorophenyl)-[1,2,4]triazolo[4,3-c]pyrimidin-3(2H)-one (150 g, 0.66 mol) in DCM/MeOH 1:1 (2 L) under N₂ atmosphere, CaCO₃ (66 g, 0.66 mol) followed by (CH₃)₃PhN⁺ Br₃⁻ (250 g, 0.66 mol) were added and the mixture was stirred at room temperature for 1 h. The reaction mixture was filtered through a Buchner funnel, rinsed with small portions of MeOH/DCM (1:1) and dried under vacuum to afford Intermediate 26, 5-amino-8-bromo-7-(4-fluorophenyl)-[1,2,4]triazolo[4,3-c]pyrimidin-3(2H)-one (120 g, 60%) as light brown solid.

LCMS (Method C): m/z 323 (M+H)⁺ (ES⁺), at 1.87 min, UV active.

¹H NMR: (400 MHz, DMSO-d6) δ: 12.58 (s, 1H), 8.19 - 8.01 (m, 2H), 7.70 - 7.67 (m, 2 H), 7.32 - 7.27 (m, 2H).

### Synthetic Route 4: Typical Procedure for the Preparation of Alkylated Triazolopyrimidinones Via Tosylation And Displacement Reaction

Intermediate 27: 5-amino-8-bromo-7-(4-fluorophenyl)-2-((5-methyloxazol-4-yl)methyl)-[1,2,4]triazolo[4,3-c]pyrimidin-3(2H)-one

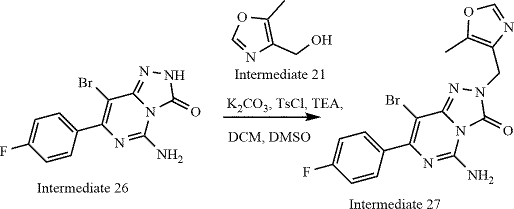

To a solution of tosyl chloride (2.67 g, 14.0 mmol), TEA (5.42 mL, 37.62 mmol) and DMAP (0.197 g, 1.617 mmol) in DCM (15 mL) at 0° C., (5-methyloxazol-4-yl)methanol (1.46 g, 12.9 mmol) was added and the resultant reaction mixture was stirred at room temperature for 30 min. The reaction mixture was partitioned between DCM (20 mL) and water (20 mL). The organic layer was separated and concentrated under reduced pressure to obtain tosylated intermediate. The tosylated intermediate was taken in DMSO (30 mL) and 5-amino-8-bromo-7-(4-fluorophenyl)-[1,2,4]triazolo[4,3-c]pyrimidin-3(2H)-one (3.5 g, 10.82 mmol) and K₂CO₃ (4.47 g, 32.3 mmol) were added and the reaction mixture was heated to 80° C. for 2 h. The reaction mixture was partitioned between EtOAc (30 mL) and water (30 mL). The organic layer was separated and concentrated under reduced pressure. The crude product was purified by Biotage-Isolera using 25 g silica snap and eluted with 0-100% EtOAc in pet-ether gradient to afford Intermediate 27, 5-amino-8-bromo-7-(4-fluorophenyl)-2-((5-methyloxazol-4-yl)methyl)-[1,2,4]triazolo[4,3-c]pyrimidin-3(2H)-one as an off white solid.

LCMS (Method C): m/z 419 (M+H)⁺ (ES⁺), at 2.20 min, UV active.

¹H NMR: (400 MHz, DMSO-d6) δ: 8.19 (s, 1H), 7.79-7.61 (m, J = 7.6, 2H), 7.38-7.28 (m, 2H), 4.91 (s, 2H), 3.77 (s, 3H). Exchangeable —NH₂ protons were not observed.

### Synthetic Route 5: Typical Procedure For The Preparation of Pyridyl Boronate Esters

Intermediate 32: 2-methoxy-6-methyl-4-(4,4,5,5-tetramethyl-1,3,2-dioxaborolan-2-yl)pyridine

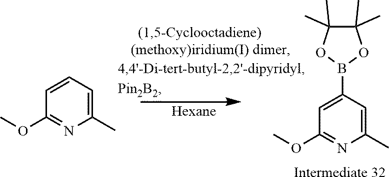

To a degassed solution of (1,5-Cyclooctadiene)(methoxy)iridium(I) dimer (108 mg, 0.16 mmol), 4,4′-Di-tert-butyl-2,2′-dipyridyl (54 mg, 0.20 mmol) in dry hexane was added Bispinacolato diborane (1.2 g, 4.87 mmol) and heated at 60° C. for 10 min. 2-methoxy-6-methylpyridine (500 mg, 0.4.05 mmol) was added to the reaction mixture and heated at 60° C. for 14 h. The reaction was concentrated under reduced pressure to afford crude Intermediate 32, 2-methoxy-6-methyl-4-(4,4,5,5-tetramethyl-1,3,2-dioxaborolan-2-yl)pyridine (800 mg) as a brown gum, which was used in the next step without further purification.

### Synthetic Route 6: Typical Procedure For The Preparation of Triazolopyrimidine Analogues Via Suzuki Coupling, Utilising SEM-Protection

Intermediate 33: methyl 4-(5-amino-3-oxo-7-phenyl-2,3-dihydro-[1,2,4]triazolo[4,3-c]pyrimidin-8-yl)-6-methylpicolinate

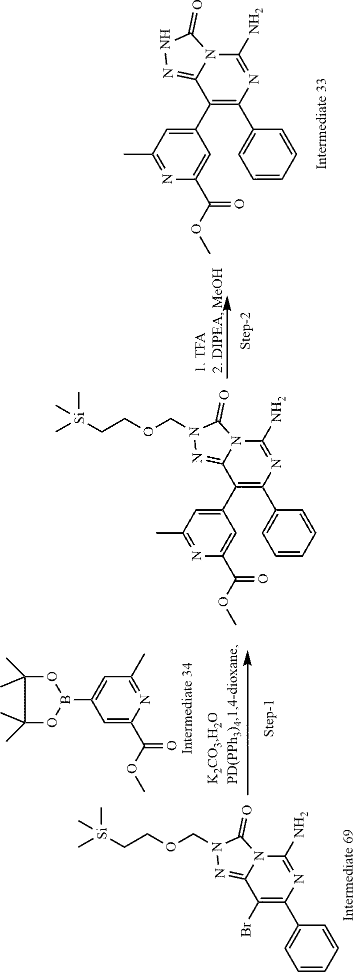

Step 1: Prepared in a similar fashion to Synthetic Route a (see below), step 2, using Intermediate 34, to afford methyl 4-(5-amino-3-oxo-7-phenyl-2-((2-(trimethylsilyl)ethoxy)methyl)-2,3-dihydro-[1,2,4]triazolo[4,3-c]pyrimidin-8-yl)-6-methyl picolinate (6 g, 64%) as a yellow solid.

LCMS (Method A): m/z 507 (M+H)⁺ (ES⁺), at 2.46 min, UV active.

¹H NMR: (400 MHz, DMSO-d6) δ: 7.67 (s, 1H), 7.32-7.27 (m, 6H), 5.14 (s, 2H), 3.81 (s, 3H), 3.64 (t, J = 7.8 Hz, 2H), 2.40 (s, 3H), 0.89 (t, J = 7.8 Hz, 2H), 0.01 (s, 9H). Exchangeable —NH2 protons were not observed.

Step 2: A solution of methyl 4-(5-amino-3-oxo-7-phenyl-2-((2-(trimethylsilyl)ethoxy)methyl)-2,3-dihydro-[1,2,4]triazolo[4,3-c]pyrimidin-8-yl)-6-methylpicolinate (1 g, 1.9 mmol) in TFA (15 mL) was stirred at room temperature for 30 minutes. After the completion of starting material, monitored by TLC, reaction mixture was concentrated under reduced pressure. The residue obtained was dissolved in MeOH (20 mL), DIPEA (1.7 mL, 9.8 mmol) was added and the resultant reaction mixture was heated to 60° C. for 4 h. The precipitate was collected by filtration, washed with MeOH (2x2 mL) and dried under vacuum to afford Intermediate 33, methyl 4-(5-amino-3-oxo-7-phenyl-2,3-dihydro-[1,2,4]triazolo[4,3-c]pyrimidin-8-yl)-6-methylpicolinate (0.55 g, 67%) as a yellow solid.

LCMS (Method A): m/z 377 (M+H)⁺ (ES⁺), at 1.65 min, UV active.

¹H NMR: (400 MHz, DMSO-d6) δ: 12.46 (s, 1H), 8.41 (s, 1H), 7.70 (s, 1H), 7.32-7.25(m, 6H), 3.85 (s, 3H), 2.47 (s, 3H). One of the exchangeable —NH₂ protons was not observed.

### Synthetic Route 7: Typical Procedure For The Alkylation of Amines

Intermediate 55: (S)-(1-(2-methoxyethyl)pyrrolidin-2-yl)methanol

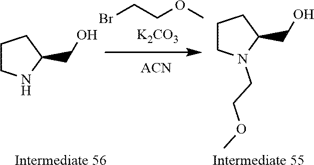

To a suspension of (S)-pyrrolidin-2-ylmethanol (400 mg, 3.96 mmol) and K₂CO₃ (1.09 g, 7.92 mmol) in MeCN (20 mL) at room temperature was added 1-bromo-2-methoxyethane (0.66 g, 4.75 mmol) and the suspension was heated at 80° C. for 15 h. The reaction mixture was partitioned between EtOAc (50 mL) and H₂O (50 mL). The organic layer was separated, dried over anhydrous Na₂SO₄ and concentrated to afford Intermediate 55, (S)-(1-(2-methoxyethyl)pyrrolidin-2-yl)methanol (400 mg, 63%) as a yellow gum.

¹H NMR: (400 MHz, DMSO-d6) δ: 4.33 (s, 1H), 3.41 - 3.37 (m, 3H), 3.23 (s, 3H), 3.20 -3.19 (m, 1H), 3.01 - 2.93 (m, 2H), 2.43 - 2.42 (m, 2H), 2.19 - 2.16 (m, 1H), 1.77 - 1.74 (m, 1H), 1.62 - 1.51 (m, 3H).

### Synthetic Route 8: Procedure For The Preparation of Intermediate 71

Intermediate 71, 2-(5-amino-8-(2,6-dimethylpyridin-4-yl)-3-oxo-7-phenyl-[1,2,4]triazolo[4,3-c]pyrimidin-2(3H)-yl)ethyl methanesulfonate

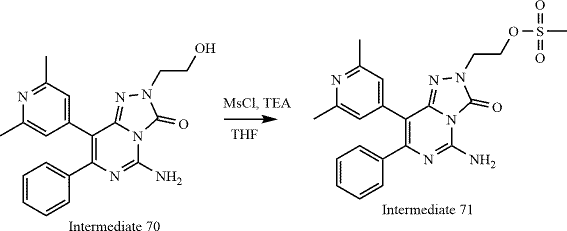

To a suspension 5-amino-8-(2,6-dimethylpyridin-4-yl)-2-(2-hydroxyethyl)-7-phenyl-[1,2,4]triazolo[4,3-c]pyrimidin-3(2H)-one (4 g, 0.01 mol) and TEA (4 mL, 0.03 mol) in THF (60 mL) at 0° C. was added methane sulfonyl chloride (1 mL, 0.012 mol) dropwise over 10 min. After the completion of starting material by TLC, reaction mixture was partitioned between EtOAc (50 mL) and brine solution (50 mL). The organic layer was separated, dried over anhydrous Na₂SO₄ and concentrated under reduced pressure. The crude product was triturated with n-Hexane (2 × 20 mL), decanted and dried under hi-vacuum to afford Intermediate 71, 2-(5-amino-8-(2,6-dimethylpyridin-4-yl)-3-oxo-7-phenyl-[1,2,4]triazolo[4,3-c]pyrimidin-2(3H)-yl)ethyl methanesulfonate (3.4 g, 70%) as a yellow solid.

LCMS (Method B): m/z 349 (M+H)⁺ (ES⁺), at 2.14 min, UV active.

¹H NMR: (400 MHz, DMSO-d6) δ: 7.62 - 7.60 (m, 2H), 7.46 - 7.44 (m, 3H), 4.83 (t, J = 6.0 Hz, 1H), 3.95-3.81 (m, 5.6 Hz, 2H), 3.72 - 3.68 (m, 2H). Exchangeable —NH₂ protons not observed.

Intermediates used in the preparation of the Examples below are listed in Table 1. Compounds were prepared according to the methods of the synthetic route indicated (“Rte.”). Where no route number or data is shown, commercially available materials were used. LCMS and ¹H NMR data are shown for purified products (or indicated as ‘used crude’ if no purification was performed). In some cases, intermediates used for the preparation of another intermediate are shown in parentheses; for example, Intermediate 28 was prepared via synthetic route 4, using Intermediate 26 and Intermediate 29.

TABLE 1

Intermediates

| Int. | Rte. | Name | Data |
|---|---|---|---|
| 1 | 1 | 5-amino-8-bromo-7-phenyl-[1,2,4]triazolo[4,3-c]pyrimidin-3(2H)-one | LCMS (Method C): m/z 306 (M+H)⁺ (ES⁺), at 1.78 min, UV active. |
| | | | ¹H NMR: (400 MHz, DMSO-d6) δ: 12.57 (s, 1H), 7.62 - 7.60 (m, 2H), 7.45 - 7.41 (m, 3H). |
| | | | Exchangeable —NH₂ protons were not observed. |
| 2 | | 2-(1H-pyrazol-1-yl)ethan-1-ol | |
| 3 | | 2,6-dimethylpyridine-4-boronic acid pinacol ester | |
| 4 | | 2-(1-methyl-1H-imidazol-2-yl)ethan-1-ol | |
| 5 | | 2-(1H-imidazol-2-yl)ethan-1-ol | |
| 6 | 2 | 5-amino-8-(2,6-dimethylpyridin-4-yl)-7-phenyl-[1,2,4]triazolo[4,3-c]pyrimidin-3(2H)-one | LCMS (Method A): m/z 333 (M+H)⁺ (ES⁺), at 1.98 min, UV active. |
| | | | ¹H NMR: (400 MHz, DMSO-d6) δ: 12.25 (s, 1H), 8.14 (s, 2H), 7.29 - 7.25 (m, 5H), 6.82 (s, 2H), 2.29 (s, 6H). |
| 7 | | 2-(1-ethyl-1H-pyrazol-5-yl)ethan-1-ol | |
| 8 | | 1-(thiophen-2-yl)ethan-1-ol | |
| 9 | | Oxazol-2-ylmethanol | |
| 10 | | 4-Oxazolemethanol | |
| 11 | | (1-methyl-1H-imidazol-2-yl)methanol | |
| 12 | | (1-methyl-1H-pyrazol-5-yl)methanol | |
| 13 | | isoxazol-3-ylmethanol | |
| 14 | | (5-methylisoxazol-3-yl)methanol | |
| 15 | | (2-methyloxazol-4-yl)methanol | |
| 16 | | (1-methyl-1H-pyrazol-3-yl)methanol | |
| 17 | | (5-methyl-1,3,4-oxadiazol-2-yl)methanol | |
| 18 | | (1-methyl-1H-imidazol-5-yl)methanol | |
| 19 | | 3-(Bromomethyl)-4-methyl-1,2,5-oxadiazole | |
| 20 | | (3-methyl-1,2,4-oxadiazol-5-yl)methanol | |
| 21 | | 5-methyloxazol-4-yl)methanol | |
| 22 | | (1H-imidazol-5-yl)methanol | |
| 23 | | (1H-imidazol-2-yl)methanol | |
| 24 | | (1H-pyrazol-5-yl)methanol | |
| 25 | | (4-methyl-4H-1,2,4-triazol-3-yl)methanol | |
| 26 | 3 | 5-amino-8-bromo-7-(4-fluorophenyl)-[1,2,4]triazolo[4,3-c]pyrimidin-3(2H)-one | LCMS (Method C): m/z 323 (M+H)⁺ (ES⁺), at 1.87 min, UV active. |
| | | | ¹H NMR: (400 MHz, DMSO-d6) δ: 12.58 (s, 1H), 8.19 - 8.01 (m, 2H), 7.70 - 7.67 (m, 2H), 7.32 - 7.27 (m, 2H). |
| 27 | 4 | 5-amino-8-bromo-7-(4-fluorophenyl)-2-((5-methyloxazol-4-yl)methyl)-[1,2,4]triazolo[4,3-c]pyrimidin-3(2H)-one | LCMS (Method C): m/z 419 (M+H)⁺ (ES⁺), at 2.20 min, UV active. |
| | | | ¹H NMR: (400 MHz, DMSO-d6) δ: 8.19 (s, 1H), 7.79-7.61 (m, J = 7.6, 2H), 7.38-7.28 (m, 2H), 4.91 (s, 2H), 3.77 (s, 3H). |
| | | | Exchangeable —NH₂ protons were not observed. |
| 28 | 4 | 5-amino-8-bromo-2-((2,5-dimethyloxazol-4-yl)methyl)-7-(4-fluorophenyl)-[1,2,4]triazolo[4,3-c]pyrimidin-3(2H)-one | LCMS (Method C): m/z 433 (M+H)⁺ (ES⁺), at 2.24 min, UV active. |
| | (26, 29) | | ¹H NMR: δ: 7.69-7.66 (m, 2H), 7.32-7.27 (m, 2H), 4.83 (s, 2H), 2.32 (s, 6H). |
| | | | Exchangeable —NH₂ protons were not observed. |
| 29 | | (2,5-dimethyloxazol-4-yl)methanol | |
| 30 | | [1-benzyl-3-(3-methoxyphenyl)-1H-pyrazol-4-yl]methanol | |
| 31 | g | 5-amino-8-bromo-2-((1-methyl-1H-imidazol-2-yl)methyl)-7-phenyl-[1,2,4]triazolo[4,3-c]pyrimidin-3(2H)-one | LCMS (Method A): m/z 400 (M+H)⁺ (ES⁺), at 2.21 min, UV active. |
| | (11) | | ¹H NMR: (400 MHz, DMSO-d6) δ: 7.61 (s, 2H), 7.47-7.44 (m, 5H), 7.43 (s, 1H), 6.82 (s, 1H), 5.11 (s, 2H), 3.72 (s, 3H). |
| 32 | 5 | 2-methoxy-6-methyl-4-(4,4,5,5-tetramethyl-1,3,2-dioxaborolan-2-yl)pyridine | Used crude |
| 33 | 6 | methyl 4-(5-amino-3-oxo-7-phenyl-2,3-dihydro-[1,2,4]triazolo[4,3-c]pyrimidin-8-yl)-6-methylpicolinate | LCMS (Method A): m/z 377 (M+H)⁺ (ES⁺), at 1.65 min, UV active. |
| | | | ¹H NMR: (400 MHz, DMSO-d6) δ: 12.46 (s, 1H), 8.41 (s, 1H), 7.70 (s, 1H), 7.32-7.25 (m, 6H), 3.85 (s, 3H), 2.47 (s, 3H). |
| | | | One of the exchangeable —NH₂ protons was not observed. |
| 34 | 5 | methyl 6-methyl-4-(4,4,5,5-tetramethyl-1,3,2-dioxaborolan-2-yl)picolinate | Used crude |
| | (35) | | |
| 35 | | Methyl 6-methylpyridine-2-carboxylate | |
| 36 | | 4-(chloromethyl)oxazole | |
| 37 | | (1,4-dimethyl-1H-imidazol-5-yl)methanol | |
| 38 | 2 | methyl 4-(5-amino-3-oxo-7-(phenyl-d5)-2,3-dihydro-[1,2,4]triazolo[4,3-c]pyrimidin-8-yl)-6-methylpicolinate | ¹H NMR: (400 MHz, DMSO-d6) δ: 12.45 (s, 1H), 8.32 (s, 2H), 7.79 (s, 1H), 7.24 (s, 1H), 3.90 (s, 3H), 2.38 (s, 3H). |
| | (39, 34) | | |
| 39 | 1 | 5-amino-8-bromo-7-(phenyl-d5)-[1,2,4]triazolo[4,3-c]pyrimidin-3(2H)-one | ¹H NMR: (400 MHz, DMSO-d6) δ: 12.57 (s, 1H), 8.30 (s, 1H), 7.59 (s, 1H). |
| | (40) | | |
| 40 | | (phenyl-d5)boronic acid | |
| 41 | | (4-methyloxazol-5-yl)methanol | |
| 42 | 2 | methyl 4-(5-amino-7-(4-fluorophenyl)-3-oxo-2,3-dihydro-[1,2,4]triazolo[4,3-c]pyrimidin-8-yl)-6-methylpicolinate | LCMS (Method C): m/z 395 (M+H)⁺ (ES⁺), at 1.51 min, UV active. |
| | (26, 34) | | ¹H NMR: (400 MHz, DMSO-d6) δ: 12.45 (s, 1H), 8.41 (s, 2H), 7.68 (s, 1H), 7.32-7.26 (m, 3H), 7.11 (m, 2H), 3.81 (s, 3H), 2.40 (s, 3H). |
| 43 | | 3-(Chloromethyl)-1-methyl-1H-pyrazole | |
| 44 | 5 | methyl 6-chloro-4-(4,4,5,5-tetramethyl-1,3,2-dioxaborolan-2-yl)picolinate | Used crude |
| | (45) | | |
| 45 | | 2-Pyridinecarboxylic acid, 6-chloro-,methyl ester | |
| 46 | 5 | methyl 6-methoxy-4-(4,4,5,5-tetramethyl-1,3,2-dioxaborolan-2-yl)picolinate | Used crude |
| | (47) | | |
| 47 | | methyl 6-methoxypicolinate | |
| 48 | 5 | methyl 4-(4,4,5,5-tetramethyl-1,3,2-dioxaborolan-2-yl)-6-(trifluoromethyl)picolinate | Used crude |
| | (49) | | |
| 49 | | methyl 6-(trifluoromethyl)picolinate | |
| 50 | | tert-butyl 3-hydroxypyrrolidine-1-carboxylate | |
| 51 | | 1-Methyl-3-pyrrolidinol | |
| 52 | | tert-butyl 2-(hydroxymethyl)pyrrolidine-1-carboxylate | |
| 53 | | (2R)-1-Methyl-2-pyrrolidinemethanol | |
| 54 | | (2S)-1-Methyl-2-pyrrolidinemethanol | |
| 55 | 7 | (S)-(1-(2-methoxyethyl)pyrrolidin-2-yl)methanol | ¹H NMR: (400 MHz, DMSO-d6) δ: 4.33 (s, 1H), 3.41 - 3.37 (m, 3H), 3.23 (s, 3H), 3.20 - 3.19 (m, 1H), 3.01 - 2.93 (m, 2H), 2.43 - 2.42 (m, 2H), 2.19 - 2.16 (m, 1H), 1.77 -1.74 (m, 1H), 1.62 - 1.51 (m, 3H). |
| 56 | | (S)-pyrrolidin-2-ylmethanol | |
| 57 | 7 | (R)-(1-(2-methoxyethyl)pyrrolidin-2-yl)methanol | ¹H NMR: (400 MHz, DMSO-d6) δ: 4.33 (s, 1H), 3.35 - 3.33 (m, 3H), 3.27 (s, 3H), 3.23 - 3.17 (m, 1H), 3.03 - 2.93 (m, 2H), 2.43 - 2.34 (m, 2H), 2.19 - 2.16 (m, 1H), 1.80 - 1.71 (m, 1H), 1.70 - 1.46 (m, 3H). |
| | (58) | | |
| 58 | | (R)-pyrrolidin-2-ylmethanol | |
| 59 | | 1-(tert-butyl) 2-methyl (2S,4R)-4-hydroxypyrrolidine-1,2-dicarboxylate | |
| 60 | | 1-(tert-butyl) 2-methyl (2R,4R)-4-hydroxypyrrolidine-1,2-dicarboxylate | |
| 61 | | 2-amino-1-(oxolan-3-yl)ethan-1-ol | |
| 62 | | (R)-(tetrahydrofuran-2-yl) methanol | |
| 63 | | (S)-(tetrahydrofuran-2-yl)methanol | |
| 64 | | 1-(tert-butyl) 2-methyl (2R,4S)-4-hydroxypyrrolidine-1,2-dicarboxylate | |
| 65 | | 1-(tert-butyl) 2-methyl (2S,4S)-4-hydroxypyrrolidine-1,2-dicarboxylate | |
| 66 | | 1-Pyrrolidineethanol | |
| 67 | | 2-fluoro-6-methylpyridine | |
| 68 | 5 | 2-fluoro-6-methyl-4-(4,4,5,5-tetramethyl-1,3,2-dioxaborolan-2-yl)pyridine | Used crude |
| | (67) | | |
| 69 | | 5-amino-8-bromo-7-phenyl-2-((2-(trimethylsilyl)ethoxy)methyl)-[1,2,4]triazolo[4,3-c]pyrimidin-3(2H)-one | LCMS (Method B): m/z 436 (M+H)⁺ (ES⁺), at 3.25 min, UV active. ¹H NMR: (400 MHz, DMSO-d6) δ: 8.56 (s, 2H), 7.62 (d, J = 7.1 Hz, 2H), 7.45 (d, J = 6.6 Hz, 3H), 5.18 (s, 2H), 3.66 (t, J = 8.2 Hz, 2H), 0.91 (t, J = 8.2 Hz, 2H), 0.04 (s, 9H). |
| 70 | 7 | 5-amino-8-bromo-2-(2-hydroxyethyl)-7-phenyl-[1,2,4]triazolo[4,3-c]pyrimidin-3(2H)-one | LCMS (Method B): m/z 349 (M+H)⁺ (ES⁺), at 2.14 min, UV active. |
| | (1, 78) | | ¹H NMR: (400 MHz, DMSO-d6) δ: 7.62 - 7.60 (m, 2H), 7.46 - 7.44 (m, 3H), 4.83 (t, J = 6.0 Hz, 1H), 3.95-3.81 (m, 5.6 Hz, 2H), 3.72 - 3.68 (m, 2H). |
| | | | Exchangeable —NH₂ protons not observed. |
| 71 | 8 | 2-(5-amino-8-(2,6-dimethylpyridin-4-yl)-3-oxo-7-phenyl-[1,2,4]triazolo[4,3-c]pyrimidin-2(3H)-yl)ethylmethanesulfonate | LCMS (Method A): m/z 455 (M+H)⁺ (ES⁺), at 2.34 min, UV active. ¹H NMR: (400 MHz, DMSO-d6 δ: 7.28 - 7.27 (m, 5H), 6.84 (s, 2H), 4.47 (t, J = 4.5 Hz, 2 H), 4.13 (t, J = 4.5 Hz, 2H), 3.17 (s, 3H), 2.30 (s, 6H). |
| | | | Exchangeable —NH₂ protons not observed. |
| 72 | | 2-(thiophen-2-yl)pyrrolidine | |
| 73 | | 1-(pyridine-3-carbonyl)pyrrolidin-3-amine | |
| 74 | | N-methyl-1H-pyrazol-4-amine dihydrochloride | |
| 75 | | (1-{[2-(aminomethyl)phenyl]methyl}pyrrolidin-2-yl)methanol | |
| 76 | | 4,6-dichloro-5-iodopyrimidin-2-amine | |
| 77 | 1, steps 1-3 only (76, 3) | 5-amino-7-chloro-8-(2,6-dimethylpyridin-4-yl)-[1,2,4]triazolo[4,3-c]pyrimidin-3(2H)-one | LCMS (Method B): m/z 291 (M+H)⁺ (ES⁺), at 1.58 min, UV active. ¹H NMR: (400 MHz, DMSO-d6 δ: 8.43 (s, 1H), 8.04 (s, 1H), 7.25 (s, 1H), 7.11 (s, 2H), 2.44 (s, 6H). |
| 78 | | 2-Bromoethanol | |

### Synthetic Routes for Examples 1-1 to 4-1

Synthetic routes a to ae, used to prepare compounds of Examples 1-1 to 4-1, are described below. The methods of synthetic routes a to ae are illustrative of the techniques used in the preparation of other compounds, as detailed in Table 2 below.

### Synthetic Route a

Example 1-1: 5-amino-8-(2,6-dimethyl-4-pyridyl)-7-phenyl-2-(2-pyrazol-1-ylethyl)-[1,2,4]triazolo[4,3-c]pyrimidin-3-one

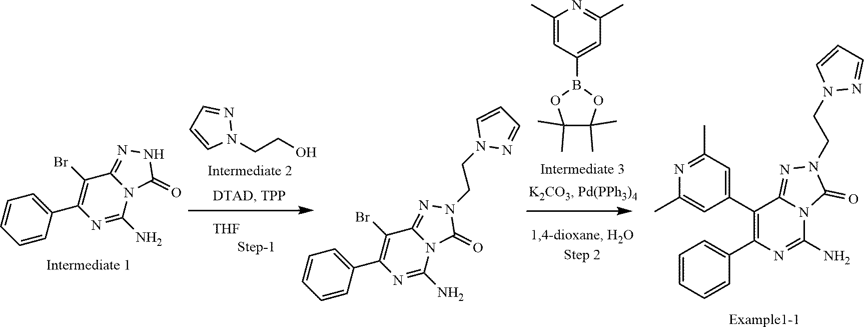

Step 1: To a suspension of Intermediate 1, 5-amino-8-bromo-7-phenyl-[1,2,4]triazolo[4,3-c]pyrimidin-3(2H)-one (0.3 g, 0.98 mmol), 2-(1H-pyrazol-1-yl)ethan-1-ol (0.10 g, 0.89 mmol) and Triphenyl phosphine (0.38 g, 1.47 mmol) in THF (10 mL) at room temperature was added di-tertiary butyl azo-dicarboxylate (0.33 g, 1.47 mmol) and the reaction mixture was stirred at room temperature for 10 min. After the completion of the reaction by TLC, the reaction mixture was concentrated under reduced pressure and the residue obtained was purified by Biotage-Isolera using 10 g silica snap and was eluted with gradient 0-50% EtOAc in Hexane to afford 2-(2-(1H-pyrazol-1-yl)ethyl)-5-amino-8-bromo-7-phenyl-[1,2,4]triazolo[4,3-c]pyrimidin-3(2H)-one as an off white solid (200 mg, 51%).

LCMS (Method B): m/z 400 (M+H)⁺ (ES⁺), at 3.52 min, UV active.

¹H NMR: (400 MHz, DMSO-d6) δ: 7.71 - 7.55 (m, 2H), 7.46 - 7.44 (m, 5H), 6.22 (s, 1H), 4.20 (t, J = 7.6 Hz, 2H), 3.72 (t, J = 7.6 Hz, 2H). Exchangeable —NH₂ protons were not observed.

Step 2: A mixture of 2-(2-(1H-pyrazol-1-yl)ethyl)-5-amino-8-bromo-7-phenyl-[1,2,4]triazolo[4,3-c]pyrimidin-3(2H)-one (0.20 g, 0.49 mmol), Intermediate 3, 2,6-dimethylpyridine-4-boronic acid pinacol ester (0.12 g, 0.54 mmol) and K₂CO₃ (137 mg, 0.99 mmol) in 1,4-dioxane/H₂O (4 mL/1 mL) was degassed for few minutes, Pd(PPh₃)₄ (29 mg, 0.02 mmol) was added, the vessel was sealed and heated to 120° C. for 5 h. After cooling to r.t. the reaction mixture was partitioned between H₂O (5 mL) and EtOAc (10 mL). The organic layer was separated, dried over anhydrous Na₂SO₄ and concentrated. The crude product was purified by Biotage-Isolera using 10 g silica snap and eluted with gradient 0-100% EtOAc in Hexane to afford Example 1-1,5-amino-8-(2,6-dimethyl-4-pyridyl)-7-phenyl-2-(2-pyrazol-1-ylethyl)-[1,2,4]triazolo[4,3-c]pyrimidin-3-one (35 mg, 16%) as a yellow solid.

LCMS (Method A): m/z 427 (M+H)⁺ (ES⁺), at 2.46 min, UV active

¹H NMR: (400 MHz, DMSO-d6) δ 7.68 (d, J = 2.0 Hz, 1H), 7.44 (d, J = 1.2 Hz, 1H), 7.27 - 7.24 (m, 5H), 6.77 (s, 2H), 6.28-6.01 (m, 1H), 4.42 (t, J = 6.0 Hz, 2H), 4.14 (t, J = 6.0 Hz, 2H), 2.28 (s, 6 H). Exchangeable —NH₂ protons were not observed

### Synthetic Route B

Example 1-4: 5-amino-8-(2,6-dimethyl-4-pyridyl)-7-phenyl-2-[2-(1H-tetrazol-5-yl)ethyl]-[1,2,4]triazolo[4,3-c]pyrimidin-3-one

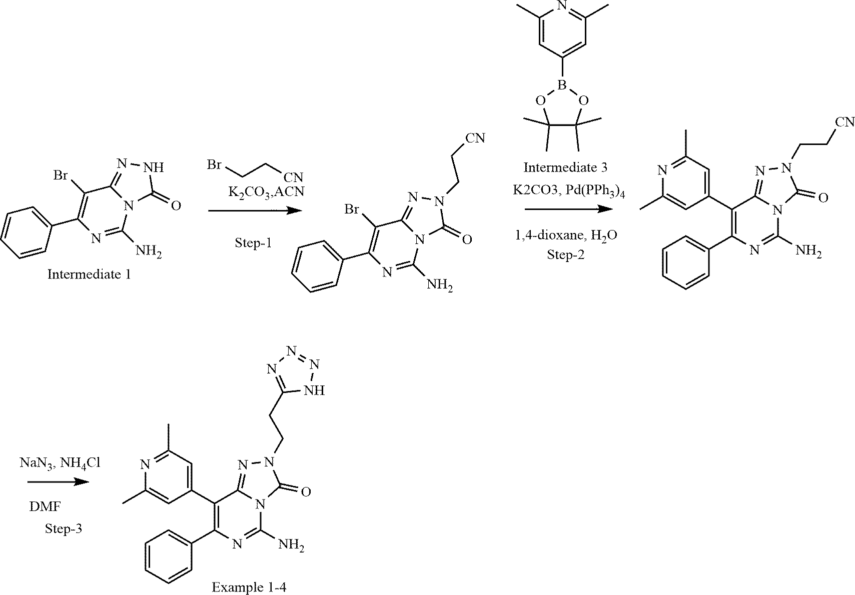

Step 1: To a suspension of 5-amino-8-bromo-7-phenyl-[1,2,4]triazolo[4,3-c]pyrimidin-3(2H)-one (300 mg, 0.98 mmol) and K₂CO₃ (406 mg, 2.9 mmol) in MeCN (3 mL) at room temperature was added 3-bromopropanenitrile (196 mg, 0.147 mmol). The reaction mixture was heated at 75° C. for 12 h. The reaction mass was cooled to room temperature and partitioned between EtOAc (10 mL) and H₂O (10 mL). Organic layer was dried over anhydrous Na₂SO₄, concentrated under reduced pressure and purified by Biotage-Isolera using 10 g silica snap and eluted with gradient 50-70% EtOAc in hexane to afford 3-(5-amino-8-bromo-3-oxo-7-phenyl-[1,2,4]triazolo[4,3-c]pyrimidin-2(3H)-yl)propanenitrile (90 mg, 25%) as a white solid.

LCMS (Method B): m/z 359 (M+H)⁺ (ES⁺), at 2.44 min, UV active.

¹H NMR: (400 MHz, DMSO-d6) δ: 8.60 - 7.90 (s, 2H), 7.64 - 7.61 (m, 2H), 7.47 - 7.44 (m, 3H), 4.13 (t, J = 6.4 Hz, 2H), 3.01 (t, J = 6.4 Hz, 2H)

Step 2: Prepared in a similar fashion to route a, step 2.

Step 3: A mixture of 3-(5-amino-8-(2,6-dimethylpyridin-4-yl)-3-oxo-7-phenyl-[1,2,4]triazolo[4,3-c]pyrimidin-2(3H)-yl)propanenitrile (202 mg, 0.52 mmol), NaN₃ (67 mg, 1.03 mmol) and ammonium chloride (69 mg, 1.3 mmol) in N,N-Dimethyl formamide (5 mL) was stirred at 120° C. for 15 h. The reaction mixture was diluted with EtOAc (20 mL) and filtered through a sintered funnel. The filtrate was concentrated under reduced pressure and the crude compound was purified by prep HPLC method (Method A). Fractions collected were concentrated under reduced pressure, the residue obtained was passed through an SCX cartridge and eluted with 2 N methanolic ammonia to afford Example 1-4, 2-(2-(1H-tetrazol-5-yl)ethyl)-5-amino-8-(2,6-dimethylpyridin-4-yl)-7-phenyl-[1,2,4]triazolo [4,3-c]pyrimidin-3(2H)-one (110 mg, 49%) as a yellow solid. The data for the title compound are in Table 2.

### Synthetic Route C

Examples 1-5 and 1-6: 5-amino-8-(2,6-dimethyl-4-pyridyl)-2-[2-(1-methyltetrazol-5-yl)ethyl]-7-phenyl-[1,2,4]triazolo[4,3-c]pyrimidin-3-one and 5-amino-8-(2,6-dimethyl-4-pyridyl)-2-[2-(2-methyltetrazol-5-yl)ethyl]-7-phenyl-[1,2,4]triazolo[4,3-c]pyrimidin-3-one

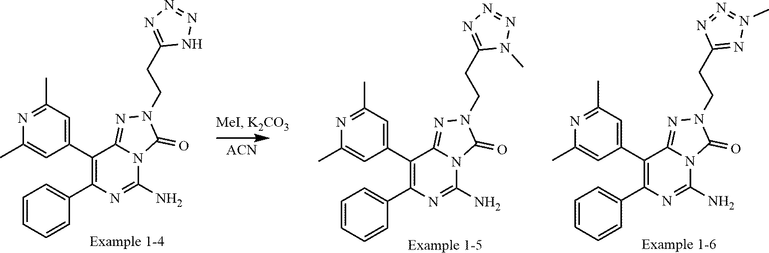

To a suspension of 2-(2-(1H-tetrazol-5-yl)ethyl)-5-amino-8-(2,6-dimethylpyridin-4-yl)-7-phenyl-[1,2,4]triazolo[4,3-c]pyrimidin-3(2H)-one (80 mg, 0.18 mmol) and K₂CO₃ (74 mg, 0.54 mmol) in MeCN (10 mL) at room temperature was added MeI (30 mg, 0.2 mmol) and the reaction was stirred at room temperature for 1 h. The reaction was filtered and concentrated. The crude products were purified by prep HPLC method (Method A). The first eluting peak was concentrated under reduced pressure and partitioned between 15% MeOH in DCM (10 mL) and 10% NaHCO₃ solution (10 mL). The organic layer was separated, dried over anhydrous Na₂SO₄, concentrated under reduced pressure and dried under hi-vacuum to afford Example 1-5, 5-amino-8-(2,6-dimethyl-4-pyridy1)-2-[2-(1-methyltetrazo1-5-y1)ethy1]-7-pheny1-[1,2,4]triazolo[4,3-c]pyrimidin-3-one (22 mg, 26%) as a yellow solid. The data for the title compound are in Table 3. The structure of this compound was confirmed by NOE study. The second eluting peak from the HPLC was concentrated under reduced pressure and partitioned between EtOAc (10 mL) and 10% NaHCO₃ solution (10 mL). The organic layer was separated, dried over anhydrous Na₂SO₄, concentrated under reduced pressure and dried under hi-vacuum to afford Example 1-6, 5-amino-8-(2,6-dimethyl-4-pyridyl)-2-[2-(2-methyltetrazol-5-yl)ethyl]-7-phenyl-[1,2,4]triazolo[4,3-c]pyrimidin-3-one (13 mg, 16%) as yellow solid. The data for the title compound are in Table 2.

### Synthetic Route D: Typical Procedure For The Preparation of Alkylated Triazolopyrimidinones Via Mitsunobu Reaction

Example 1-7: 5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one

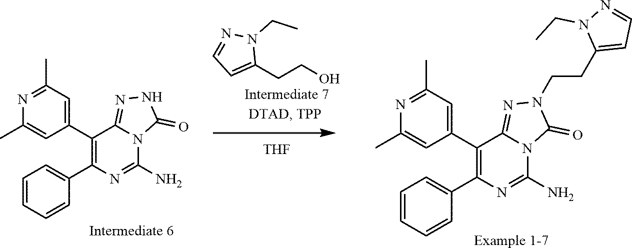

To a mixture of 5-amino-8-bromo-7-phenyl-[l,2,4]triazolo[4,3-c]pyrimidin-3(2H)-one (75 mg, 0.25 mmol), 2-(1-ethyl-1H-pyrazol-5-yl)ethan-1-ol (38 mg, 0.27 mmol) and triphenylphoshine (77 mg, 0.29 mmol) in THF (3 mL) was added di-tert-butyl-azodicarboxylate (77 mg, 0.27 mmol) and the reaction stirred at room temperature for 18 h. The mixture was concentrated under reduced pressure and the crude product was purified by prep HPLC (method E) to afford Example 1-7, 5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one (7.7 mg, 7%). The data for the title compound are in Table 2.

### Synthetic Route E: Typical Procedure For The Preparation of Alkylated Triazolopyrimidinones Via Alcohol Mesylation Followed By An Alkylation Reaction

Example 1-9, 5-amino-8-(2,6-dimethyl-4-pyridyl)-2-(oxazol-2-ylmethyl)-7-phenyl-[1,2,4]triazolo[4,3-c]pyrimidin-3-one

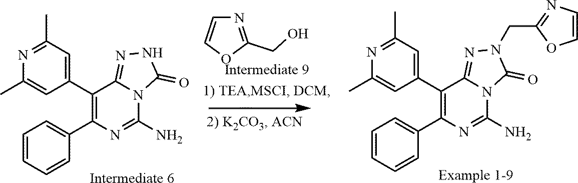

To a solution of oxazol-2-ylmethanol (36 mg, 0.36 mmol) and TEA (103 mg, 0.90 mmol) in DCM (10 mL) at 0° C. was added mesyl chloride (56 mg, 0.45 mmol) and the reaction mixture was stirred at room temperature for 30 min. The reaction mixture was partitioned between DCM (20 mL) and water (20 mL). The organic layer was separated, dried over anhydrous Na₂SO₄ and concentrated under reduced pressure to obtain mesylated intermediate. The mesylated intermediate was taken in MeCN (20 mL) and 5-amino-8-(2,6-dimethylpyridin-4-yl)-7-phenyl-[1,2,4]triazolo[4,3-c]pyrimidin-3(2H)-one (100 mg, 0.30 mmol), K₂CO₃ (125 mg, 0.90 mmol) were added and heated to 80° C. for 16 h in sealed vial. The reaction mixture was partitioned between EtOAc (20 mL) and water (20 mL). The organic layer was separated, dried over anhydrous Na₂SO₄ and concentrated under reduced pressure. The crude product was purified by Prep-HPLC (Method A). Fractions were concentrated and diluted with EtOAc (10 mL), washed with 10% sodium bicarbonate solution (10 mL). The organic layer was separated, dried over anhydrous Na₂SO₄ and concentrated to dryness to afford Example 1-9, 5-amino-8-(2,6-dimethyl-4-pyridyl)-2-(oxazol-2-ylmethyl)-7-phenyl-[1,2,4]triazolo[4,3-c]pyrimidin-3-one (21 mg, 0.16%) as yellow solid. The data for the title compound are in Table 2.

### Synthetic Route F: Typical Procedure For The Preparation Of Alkylated Triazolopyrimidinones Via Alcohol Tosylation Followed By An Alkylation Reaction

Example 1-12: 5-amino-8-(2,6-dimethyl-4-pyridyl)-2-[(2-methylpyrazol-3-yl)methyl]-7-phenyl-[1,2,4]triazolo[4,3-c]pyrimidin-3-one

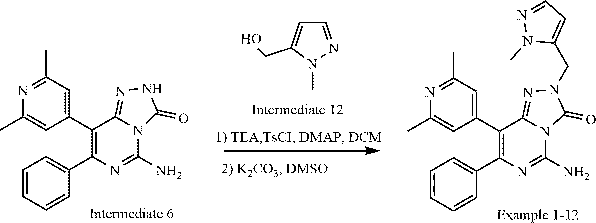

To a solution of N,N-dimethylpyridin-4-amine (3.6 mg, 0.03 mmol), TEA (45.6 mg, 0.45 mmol) and tosyl chloride (63.1 mg, 0.33 mmol) in DCM (10 mL) at 0° C. was added (1-methyl-1H-pyrazol-5-yl)methanol (40.4 mg, 0.36 mmol) and the reaction mixture was stirred at room temperature for 1 h. After the completion of starting material, monitored by TLC, reaction mixture was partitioned between DCM (20 mL) and water (20 mL). The organic layer was separated, dried over anhydrous Na₂SO₄ and concentrated under reduced pressure to obtain tosylated intermediate. The tosylated intermediate was taken in DMSO (10 mL), 5-amino-8-(2,6-dimethylpyridin-4-yl)-7-phenyl-[1,2,4]triazolo[4,3-c]pyrimidin-3(2H)-one (100 mg, 0.30 mmol) and K₂CO₃ (125 mg, 0.90 mmol) were added and heated to 80° C. for 16 h in sealed vial. After the completion of starting material, monitored by TLC, reaction mixture was partitioned between EtOAc (20 mL) and water (20 mL). The organic layer was separated, dried over anhydrous Na₂SO₄ and concentrated under reduced pressure. The crude product was purified by Prep-HPLC (Method A). Fractions were concentrated under reduced pressure and the residue obtained was diluted with EtOAc (10 mL), washed with 10% sodium bicarbonate solution (10 mL). The organic layer was separated, dried over anhydrous Na₂SO₄ and concentrated to afford Example 1-12, 5-amino-8-(2,6-dimethylpyridin-4-yl)-2-((1-methyl-1H-pyrazol-5-yl)methyl)-7-phenyl-[1,2,4]triazolo[4,3-c]pyrimidin-3(2H)-one (15 mg, 11%) as yellow solid. The data for the title compound are in Table 2.

### Synthetic Route G: Typical Procedure For The Preparation of Alkylated Triazolopyrimidinones Via Alcohol Chlorination Followed By An Alkylation Reaction

Example 1-16: 5-amino-8-(2,6-dimethyl-4-pyridyl)-2-[(1-methylpyrazol-3-yl)methyl]-7-phenyl-[1,2,4]triazolo[4,3-c]pyrimidin-3-one

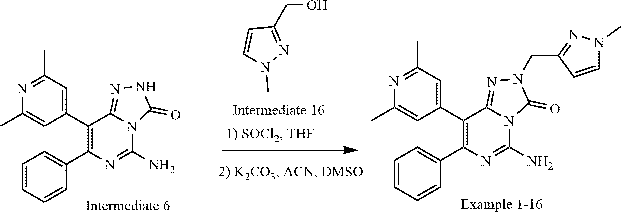

To a solution of (1-methyl-1H-pyrazol-3-yl)methanol (41 mg, 0.36 mmol) in THF (10 mL) at 0° C. was added SOCl₂ (1 mL) dropwise and stirred at 60° C. for 2 h. (Alternatively, toluene may be used as solvent and the reaction heated to 110° C.). After the completion of starting material, monitored by TLC, the reaction mixture was partitioned between EtOAc (20 mL) and 10% sodium bicarbonate solution (10 mL). The organic layer was separated, dried over anhydrous Na₂SO₄ and concentrated under reduced pressure to obtain Chloro-intermediate. This Chloro- intermediate was taken in MeCN/DMSO (20 mL/1 mL), 5-amino-8-(2,6-dimethylpyridin-4-yl)-7-phenyl-[1,2,4]triazolo[4,3-c]pyrimidin-3(2H)-one (100 mg, 0.30 mmol) and K₂CO₃ (125 mg, 0.90 mmol) were added and heated to 80° C. for 16 h in sealed vial. After the completion of starting material, monitored by TLC, reaction mixture was partitioned between EtOAc (20 mL) and water (20 mL). The organic layer was separated, dried over anhydrous Na₂SO₄ and concentrated under reduced pressure. The crude product was purified by Prep-HPLC (Method A). Fractions were concentrated and the residue obtained was diluted with EtOAc (10 mL) and washed with 10% sodium bicarbonate solution (10 mL). The organic layer was separated, dried over anhydrous Na₂SO₄ and concentrated to afford Example 1-16, 5-amino-8-(2,6-dimethyl-4-pyridyl)-2-[(1-methylpyrazol-3-yl)methyl]-7-phenyl-[1,2,4]triazolo[4,3-c]pyrimidin-3-one (18 mg, 14%) as yellow solid. The data for the title compound are in Table 2.

### Synthetic Route H: Typical Procedure for the Preparation of Alkylated Triazolopyrimidinones via an Alkylation Reaction

Example 1-19: 5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one

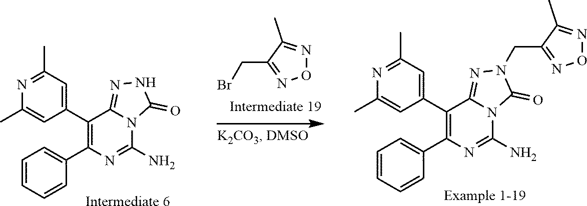

To a solution of 5-amino-8-(2,6-dimethylpyridin-4-yl)-7-phenyl-[1,2,4]triazolo[4,3-c]pyrimidin-3(2H)-one ( 50 mg, 0.15) and 3-(bromomethyl)-4-methyl-1,2,5-oxadiazole (26.6 mg, 0.15 mmol) in DMSO (5 mL) at room temperature was added K₂CO₃ (62.3 mg, 0.45 mmol) and the resultant reaction mixture was heated to 80° C. for 1 h. After the completion of starting material, monitored by TLC, reaction mixture was partitioned between EtOAc (10 mL) and water (10 mL). The organic layer was separated, dried over anhydrous Na₂SO₄ and concentrated under reduced pressure to obtain crude product. The crude compound was purified by Biotage-Isolera using 10 g silica gel snap and was eluted with 0-100% EtOAc in Pet ether gradient to afford Example 1-19, 5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one (22 mg, 34%) as yellow solid. The data for the title compound are in Table 2.

### Synthetic Route I

Example 1-20: 5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one

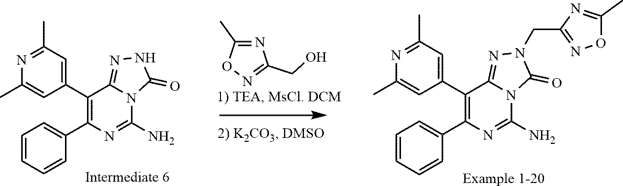

To a solution of (3-methyl-1,2,4-oxadiazol-5-yl)methanol (90 mg, 0.783 mmol) and TEA (0.4 mL, 3.012 mmol) in DCM (10 mL) at 0° C. was added mesyl chloride (0.07 mL, 0.903 mmol) and the reaction mixture was stirred at room temperature for 30 min. The reaction mixture was partitioned between DCM (20 mL) and water (10 mL). The organic layer was separated and concentrated under reduced pressure to obtain mesylated intermediate. The mesylated intermediate was taken in DMSO (10 mL) and 5-amino-8-(2,6-dimethylpyridin-4-yl)-7-phenyl-[1,2,4]triazolo[4,3-c]pyrimidin-3(2H)-one (200 mg, 0.602 mmol), K₂CO₃ (249 mg, 1.807 mmol) were added and the resultant reaction mixture was heated at 80° C. for 2 h in a sealed vial. The reaction mixture was quenched with ice cold water. The resultant solid was filtered, washed with EtOH and dried under vacuum to afford 5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one (49 mg, 0.18%) as an off white solid. The data for the title compound are in Table 2.

### Synthetic Route J

Example 1-26:5-amino-8-(2,6-dimethyl-4-pyridyl)-7-phenyl-2-(2H-tetrazol-5-ylmethyl)-[1,2,4]triazolo[4,3-c]pyrimidin-3-one

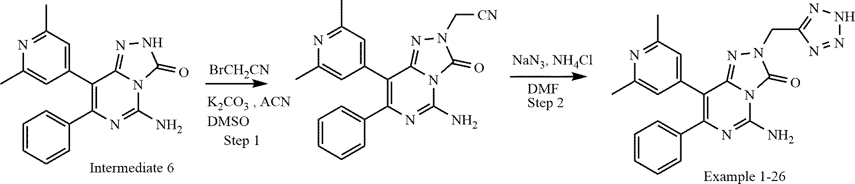

Step 1: To a solution of 5-amino-8-(2,6-dimethylpyridin-4-yl)-7-phenyl-[1,2,4]triazolo[4,3-c]pyrimidin-3(2H)-one (100 mg, 0.30 mmol) and K₂CO₃ (84 mg, 0.60 mmol) in MeCN (8 mL) and DMSO (2 mL), 2-bromoacetonitrile (36 mg, 0.30 mmol) was added dropwise and heated to 80° C. for 16 h in sealed tube. The reaction mixture was partitioned between EtOAc (20 mL) and water (20 mL), the organic layer was separated, dried over anhydrous Na₂SO₄ and concentrated under reduced pressure to afford crude 2-(5-amino-8-(2,6-dimethylpyridin-4-yl)-3-oxo-7-phenyl-[1,2,4]triazolo[4,3-c]pyrimidin-2(3H)-yl)acetonitrile (70 mg, 62%), which was taken onto the next step without further purification.

LCMS (Method A): m/z 372 (M+H)⁺ (ES⁺), at 2.15 min, UV active.

¹H NMR: (400 MHz, DMSO-d6) δ: 7.32 - 7.28 (m, 5H), 6.84 (s, 2H), 5.16 (s, 2H), 2.31 (s, 6H). Exchangeable —NH₂ protons were not observed.

Step 2: A solution of 2-(5-amino-8-(2,6-dimethylpyridin-4-yl)-3-oxo-7-phenyl-[1,2,4]triazolo[4,3-c]pyrimidin-2(3H)-yl)acetonitrile (70 mg, 0.18 mmol), NaN₃ (37 mg, 0.56 mmol) and NH₄Cl (30.2 mg, 0.56 mmol) in DMF (15 mL) was heated to 120° C. for 16 h in sealed tube. The reaction mixture was partitioned between EtOAc (20 mL) and water (20 mL), the organic layer was separated, dried over anhydrous Na₂SO₄ and concentrated. The crude was purified by Prep-HPLC (Method A). Fractions were concentrated and the residue obtained was diluted with EtOAc (10 mL) and washed with 10% sodium bicarbonate solution (10 mL). The organic layer was separated, dried over anhydrous Na₂SO₄ and concentrated to dryness to afford 5-amino-8-(2,6-dimethyl-4-pyridyl)-7-phenyl-2-(2H-tetrazol-5-ylmethyl)-[1,2,4]triazolo[4,3-c]pyrimidin-3-one (30 mg, 38%) as a yellow solid. The data for the title compound are in Table 2.

### Synthetic Route K

Example 1-27: 5-amino-8-(2,6-dimethyl-4-pyridyl)-2-(1,3,4-oxadiazol-2-ylmethyl)-7-phenyl-[1,2,4]triazolo[4,3-c]pyrimidin-3-one

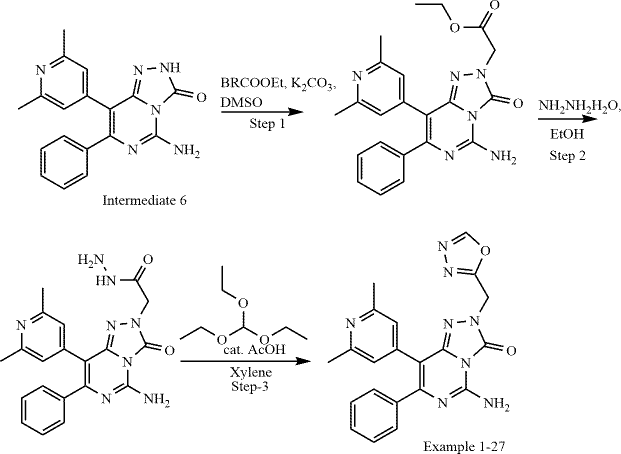

Step 1: To a solution of 5-amino-8-(2,6-dimethylpyridin-4-yl)-7-phenyl-[1,2,4]triazolo[4,3-c]pyrimidin-3(2H)-one (200 mg, 0.602 mmol) in DMSO (5 mL), K₂CO₃ (249 mg, 1.807 mmol) and ethylbromoacetate (73 mg, 0.662 mmol) were added and the resultant reaction mixture was stirred at 70° C. for 2 h. The reaction mixture was quenched with ice cold water and stirred. The precipitate was filtered and dried under reduced pressure to afford ethyl 2-(5-amino-8-(2,6-dimethylpyridin-4-yl)-3-oxo-7-phenyl-[1,2,4]triazolo[4,3-c]pyrimidin-2(3H)-yl)acetate (150 mg, 59%) as a yellow solid.

LCMS (Method A): m/z 419 (M+H)⁺ (ES⁺), at 2.00 min, UV active.

¹H NMR: (400 MHz, DMSO-d6) δ: 8.45 (s, 2H), 7.28 (s, 5H), 6.83 (d, J = 9.8 Hz, 2H), 4.73 (s, 2H), 4.19-4.14 (m, 2H), 2.29 (s, 6H), 1.22 (t, J = 14.1 Hz, 3H).

Step 2: To a solution of ethyl 2-(5-amino-8-(2,6-dimethylpyridin-4-yl)-3-oxo-7-phenyl-[1,2,4]triazolo[4,3-c]pyrimidin-2(3H)-yl)acetate (150 mg, 0.358 mmol) in EtOH (10 mL) was added hydrazine hydrate (44.8 mg, 0.897 mmol) and the resultant reaction mixture was heated at 90° C. for 16 h. The reaction mixture was evaporated under reduced pressure. The crude product was triturated with EtOAc (2×2 mL), decanted and dried under vacuum to afford 2-(5-amino-8-(2,6-dimethylpyridin-4-yl)-3-oxo-7-phenyl-[1,2,4]triazolo[4,3-c]pyrimidin-2(3H)-yl)acetohydrazide as pale green solid (75 mg, 51%), which was used without further purification.

LCMS (Method A): m/z 405 (M+H)⁺ (ES⁺), at 1.67 min, UV active.

Step 3: To a solution of 2-(5-amino-8-(2,6-dimethylpyridin-4-yl)-3-oxo-7-phenyl-[1,2,4]triazolo[4,3-c]pyrimidin-2(3H)-yl)acetohydrazide (75 mg, 0.185 mmol) in xylene (5 mL), triethylorthoformate (55 mg, 0.371 mmol) and a catalytic amount of AcOH were added and the resultant reaction mixture was heated to 130° C. for 16 h. The reaction mixture was evaporated under reduced pressure to remove the volatiles. The crude product was partitioned between EtOAc (10 mL) and water (5 mL). The organic layer was separated, washed with brine (5 mL), dried over anhydrous Na₂SO₄ and concentrated under reduced pressure. The crude compound was purified by Prep-TLC (GF254 silica coated glass plate (20x20 cm); Mobile Phase: 2% MeOH in DCM) to afford 5-amino-8-(2,6-dimethyl-4-pyridyl)-2-(1,3,4-oxadiazol-2-ylmethyl)-7-phenyl-[1,2,4]triazolo[4,3-c]pyrimidin-3-one (10 mg, 13%) as a yellow solid. The data for the title compound are in Table 2.

### Synthetic Route L

Example 1-29: 5-amino-8-(2,6-dimethyl-4-pyridyl)-2-[(5-methyl-1H-triazol-4-yl)methyl]-7-phenyl-[l,2,4]triazolo[4,3-c]pyrimidin-3-one

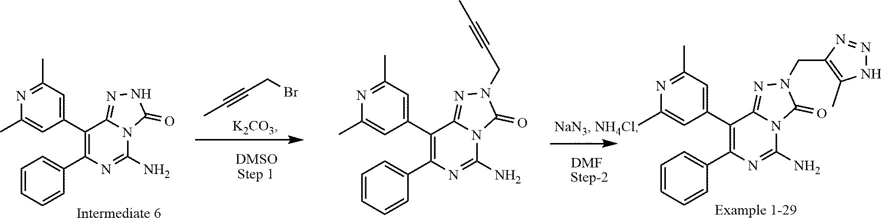

Step 1: To a stirred solution of 5-amino-8-(2-methylpyridin-4-yl)-7-phenyl-[1,2,4]triazolo[4,3-c]pyrimidin-3(2H)-one (100 mg, 0.301 mmol) in DMSO (3 mL) was added K₂CO₃ (103 mg) followed by the addition of 1-bromobut-2-yne (60 mg, 0.45 mmol). The reaction was heated to 50° C. for 1 h, then diluted with ice water and the precipitated compound was filtered and dried to afford 5-amino-2-(but-2-yn-1-yl)-8-(2,6-dimethylpyridin-4-yl)-7-phenyl-[1,2,4]triazolo[4,3-c]pyrimidin-3(2H)-one (70 mg, 63%) as yellow solid.

LCMS (Method C): m/z 385 (M+H)⁺ (ES⁺), at 1.67 min, UV active.

¹H NMR: (400 MHz, DMSO-d6) δ: 7.27-7.25 (m, 5H), 6.83 (s, 2H), 4.61 (d, J = 4.0 Hz, 2H), 2.30 (s, 6H), 1.79-1.78 (m, 3H). Exchangeable —NH₂ protons were not observed.

Step 2: A suspension of 5-amino-2-(but-2-yn-1-yl)-8-(2,6-dimethylpyridin-4-yl)-7-phenyl-[1,2,4]triazolo[4,3-c]pyrimidin-3(2H)-one (60 mg), NaN₃ (20 mg) and NH₄Cl (25 mg) in DMF (75 mL) was heated to 120° C. for 48 h. The reaction mixture was partitioned between ice water (10 mL) and EtOAc (10 mL). The organic layer was separated, dried over anhydrous Na₂SO₄ and concentrated under vacuum. The crude product was purified by prep-HPLC (Method-A). Fractions were concentrated under reduced pressure, residue obtained was partitioned between EtOAc (10 mL) and 10% NaHCO₃ solution (10 mL). The organic layer was separated, dried over anhydrous Na₂SO₄ and concentrated under reduced pressure to afford (5-amino-8-(2,6-dimethylpyridin-4-yl)-2-((5-methyl-1H-1,2,3-triazol-4-yl)methyl)-7-phenyl-[1,2,4]triazolo[4,3-c]pyrimidin-3(2H)-one (14 mg, 25%) as yellow solid. The data for the title compound are in Table 2.

### Synthetic Route M

Example 1-30: 5-amino-8-(2,6-dimethyl-4-pyridyl)-2-[(2-methyltriazol-4-yl)methyl]-7-phenyl-[1,2,4]triazolo[4,3-c]pyrimidin-3-one

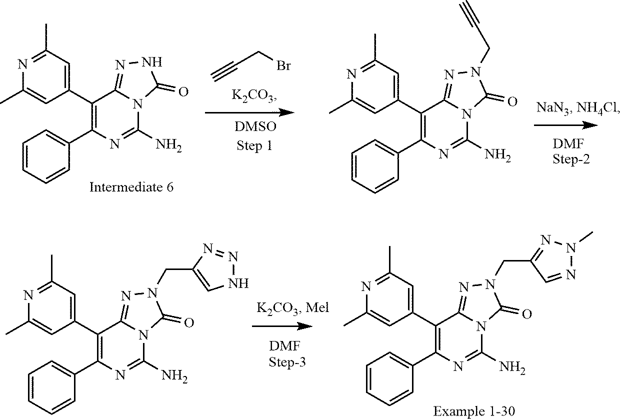

Step 1: To a stirred solution of 5-amino-8-(2,6-dimethylpyridin-4-yl)-7-phenyl-[1,2,4]triazolo[4,3-c]pyrimidin-3(2H)-one (250 mg, 0.75 mmol) and K₂CO₃ (311 mg, 2.25 mmol) in DMSO (8 mL) at 0° C., 3-bromoprop-1-yne (89.5 mg, 0.75 mmol) was added and the reaction mixture was heated to 50° C. for 1 h. The reaction mixture was diluted with ice cold water and the precipitated solid was collected by filtration, rinsed with water (10 mL) and dried under vacuum to afford 5-amino-8-(2,6-dimethylpyridin-4-yl)-7-phenyl-2-(prop-2-yn-1-yl)-[1,2,4]triazolo[4,3-c]pyrimidin-3(2H)-one (200 mg, 74%) as a yellow solid.

LCMS (Method C): m/z 371 (M+H)⁺ (ES⁺), at 1.08 min, UV active.

¹H NMR: (400 MHz, DMSO-d6) δ: 7.28-7.25 (m, 5H), 6.83 (s, 2H), 4.68 (d, J = 4.0 Hz, 2H), 3.34 (s, 1H), 2.33 (s, 6H). Exchangeable protons of —NH2 not observed.

Step 2: A suspension of 5-amino-8-(2,6-dimethylpyridin-4-yl)-7-phenyl-2-(prop-2-yn-1-yl)-[1,2,4]triazolo[4,3-c]pyrimidin-3(2H)-one (200 mg, 0.53 mmol), NaN₃(70 mg, 1.09 mmol) and NH₄Cl (85.04 mg, 1.59 mmol) in DMF (30 mL) was heated to 120° C. for 48 h. The reaction was diluted with ice cold water (10 mL) and extracted with EtOAc (15 mL). The organic layer was dried over anhydrous Na₂SO₄ and concentrated under vacuum. The crude product was purified by Biotage-Isolera using 230-400 silica mesh and eluted with 0-100% EtOAc in Hexane as a gradient to afford (2-((1H-1,2,3-triazol-4-yl)methyl)-5-amino-8-(2,6-dimethylpyridin-4-yl)-7-phenyl-[1,2,4]triazolo[4,3-c]pyrimidin-3(2H)-one (50 mg, 20%) as a yellow solid.

LCMS (Method D): m/z 414 (M+H)⁺ (ES⁺), at 2.39 min, UV active.

Step 3: To a stirred solution of 2-((1H-1,2,3-triazol-4-yl)methyl)-5-amino-8-(2,6-dimethylpyridin-4-yl)-7-phenyl-[1,2,4]triazolo[4,3-c]pyrimidin-3(2H)-one (50 mg, 0.121 mmol) and K₂CO₃ (49 mg, 0.36 mmol) in DMF (5 mL) at 0° C., Mel (0.08 ml, 0.75 mmol) was added and the reaction mixture was stirred at room temperature for 1 h. The reaction mixture was diluted with ice cold water and the precipitated solid was filtered to afford crude product. The crude material was purified by prep HPLC (Method A). Fractions were concentrated and the residue obtained was diluted with EtOAc (10 mL), washed with 10% sodium bicarbonate solution (10 mL). The organic layer was separated, dried over anhydrous Na₂SO₄ and concentrated to afford 5-amino-8-(2,6-dimethyl-4-pyridyl)-2-[(2-methyltriazol-4-yl)methyl]-7-phenyl-[1,2,4]triazolo[4,3-c]pyrimidin-3-one (10 mg, 19%) as pale a yellow solid. The data for the title compound are in Table 2.

### Synthetic Route N: Typical Procedure For The Preparation of Pyridine-N-Oxides

Example 1-34: 5-amino-8-(2,6-dimethyl-1-oxido-pyridin-1-ium-4-yl)-2-[(5-methyloxazol-4-yl)methyl]-7-phenyl-[1,2,4]triazolo[4,3-c]pyrimidin-3-one

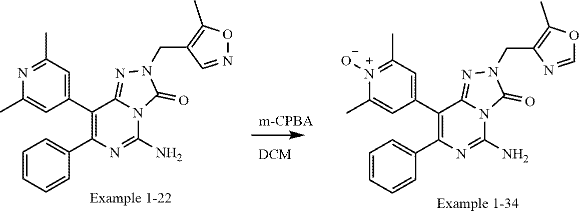

To a solution of 5-amino-8-(2,6-dimethylpyridin-4-yl)-2-((5-methyloxazol-4-yl)methyl)-7-phenyl-[1,2,4]triazolo[4,3-c]pyrimidin-3(2H)-one (95 mg, 0.22 mmol) in DCM at 0° C., m-CPBA (46 mg, 0.26 mmol) was added portion wise and the resultant reaction mixture was stirred at room temperature for 30 minutes. The reaction mixture was partitioned between EtOAc (15 mL) and water (10 mL). The organic layer was separated, dried over anhydrous Na₂SO₄ and concentrated under reduced pressure. The crude product was purified by Prep-HPLC (Method A), the fractions were concentrated and the residue obtained was diluted with EtOAc (10 mL), washed with 10% sodium bicarbonate solution (10 mL). The organic layer was separated, dried over anhydrous Na₂SO₄ and concentrated to afford 4-(5-amino-2-((5-methyloxazol-4-yl)methyl)-3-oxo-7-phenyl-2,3-dihydro-[1,2,4]triazolo[4,3-c]pyrimidin-8-yl)-2,6-dimethylpyridine 1-oxide (7.3 mg, 8%) as yellow solid. The data for the title compound are in Table 2.

### Synthetic Route O: Typical Procedure for the Preparation of Hydroxymethyl Pyridine Analogues Via Alcohol Chlorination and Alkylation Followed By Ester Reduction

Example 1-39: 5-amino-8-[2-(hydroxymethyl)-6-methyl-4-pyridyl]-2-[(5-methyloxazol-4-yl)methyl]-7-phenyl-[1,2,4]triazolo[4,3-c]pyrimidin-3-one

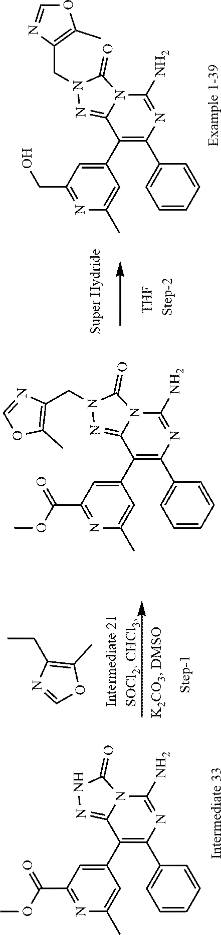

Step 1: To a solution of 5-methyloxazol-4-yl)methanol (36 mg, 0.32 mmol) in CH₃Cl (5 mL) at 0° C., thionyl chloride (0.05 mL, 0.66 mmol) was added drop wise and the resultant reaction mixture was heated to 50° C. for 60 min. After the completion of starting material, monitored by TLC, the reaction mixture was concentrated under reduced pressure to afford chlorinated intermediate. The chlorinated intermediate was taken in DMSO (2 mL) and methyl 4-(5-amino-3-oxo-7-phenyl-2,3-dihydro-[1,2,4]triazolo[4,3-c]pyrimidin-8-yl)-6-methylpicolinate (100 mg, 0.26 mmol) and K₂CO₃ (110 mg, 0.79 mmol) were added and the resultant reaction mixture was heated to 80° C. for 2 h in a sealed tube. After the completion of starting material, monitored by TLC, reaction mixture was partitioned between EtOAc (10 mL) and water (10 mL). The organic layer was separated and concentrated under reduced pressure. The crude product was purified by Biotage-Isolera using 10 g silica snap and eluted with 0-100% EtOAc in Pet-ether gradient to afford methyl 4-(5-amino-2-((5-methyloxazol-4-yl)methyl)-3-oxo-7-phenyl-2,3-dihydro-[1,2,4]triazolo[4,3-c]pyrimidin-8-yl)-6-methylpicolinate (80 mg, 66%) as yellow solid.

LCMS (Method C): m/z 472 (M+H)⁺ (ES⁺), at 1.69 min, UV active.

¹H NMR: (400 MHz, DMSO-d6) δ: 8.17 (s, 1H), 7.32 (s, 2H), 7.29-7.26 (m, 4H), 7.20 (s, 1H), 4.87 (s, 2H), 3.81 (s, 3H), 2.36 (s, 6H). Exchangeable —NH₂ protons were not observed.

Step 2: To a solution of methyl 4-(5-amino-2-((5-methyloxazol-4-yl)methyl)-3-oxo-7-phenyl-2,3-dihydro-[1,2,4]triazolo[4,3-c]pyrimidin-8-yl)-6-methylpicolinate (80 mg, 0.16 mmol) in THF (5 mL) at 0° C., lithium triethyl borohydride (1 M in THF, 0.33 mL, 0.33 mmol) was added dropwise and the reaction mixture was stirred at room temperature for 20 min. After the completion of starting material, monitored by TLC, the reaction mixture was partitioned between EtOAc (5 mL) and H₂O (5 mL). The organic layer was separated, dried over anhydrous sodium sulphate and concentrated under reduced pressure. The crude product was purified by Prep-HPLC (Method A). Fractions were concentrated and the residue obtained was diluted with EtOAc (10 mL), washed with 10% sodium bicarbonate solution (10 mL). The organic layer was separated, dried over anhydrous Na₂SO₄ and concentrated to afford 5-amino-8-(2-(hydroxymethyl)-6-methylpyridin-4-yl)-2-((5-methyloxazol-4-yl)methyl)-7-phenyl-[1,2,4]triazolo[4,3-c]pyrimidin-3(2H)-one (18 mg, 24%) as a yellow solid. The data for the title compound are in Table 2.

### Synthetic Route P: Typical Procedure For the Preparation of Hydroxymethyl Pyridine Analogues Via Alcohol Tosylation And Alkylation Followed By Ester Reduction

Example 1-40: 5-amino-8-[2-(hydroxymethyl)-6-methyl-4-pyridyl]-2-(oxazol-2-ylmethyl)-7-phenyl-[1,2,4]triazolo[4,3-c]pyrimidin-3-one

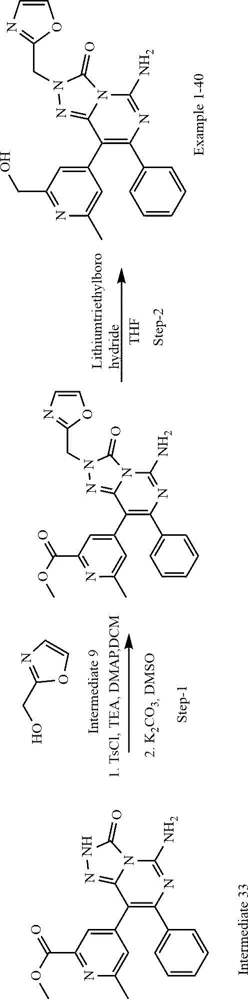

Step 1: To a solution of tosyl chloride (55.7 mg, 0.79 mmol), DMAP (3.2 mg, 0.02) and TEA (0.1 mL, 0.07 mmol) in DCM (10 mL) at 0° C. was added oxazol-2-ylmethanol (32 mg, 0.31 mmol) dissolved in DCM (0.5 mL) and the reaction mixture was stirred at room temperature for 30 min. After the completion of starting material, monitored by TLC, reaction mixture was partitioned between DCM (20 mL) and water (20 mL). The organic layer was separated and concentrated under reduced pressure to obtain tosylated intermediate. The tosylated intermediate was taken in DMSO (2 mL) and was added to a suspension of methyl 4-(5-amino-3-oxo-7-phenyl-2,3-dihydro-[1,2,4]triazolo[4,3-c]pyrimidin-8-yl)-6-methylpicolinate (100 mg, 0.26 mmol) and K₂CO₃ (110 mg, 0.29 mmol) in DMSO (2 mL) and the resultant reaction mixture was heated to 50° C. for 6 h in sealed vial. After the completion of starting material, monitored by TLC, the reaction mixture was partitioned between EtOAc (10 mL) and water (10 mL). The organic layer was separated and concentrated under reduced pressure. The crude product was purified by Biotage-Isolera using 10 g silica gel snap and was eluted with 0-100% EtOAc in Pet ether gradient to afford methyl 4-(5-amino-2-(oxazol-2-ylmethyl)-3-oxo-7-phenyl-2,3-dihydro-[1,2,4]triazolo[4,3-c]pyrimidin-8-yl)-6-methylpicolinate (70 mg, 57%) as a yellow solid.

LCMS (Method C): m/z 458 (M+H)⁺ (ES⁺), at 1.60 min, UV active.

¹H NMR: (400 MHz, DMSO-d6) δ: 8.12 (s, 1H),7.67 (s,1H) 7.27-7.21 (m, 5H), 6.82-6.76 (m, 2H), 3.80 (s, 2H), 2.95 (d, J = 5.2 Hz, 3H), 2.51 (s, 3H). Exchangeable —NH₂ Protons were not observed.

Step 2: Performed in a similar fashion to Route o, step 2 to afford 5-amino-8-[2-(hydroxymethyl)-6-methyl-4-pyridyl]-2-(oxazol-2-ylmethyl)-7-phenyl-[1,2,4]triazolo[4,3-c]pyrimidin-3-one (4.8 mg, 7.3%) as a yellow solid. The data for the title compound are in Table 2.

### Synthetic Route Q: Typical Procedure For the Preparation of Hydroxymethyl Pyridine Analogues Via Alcohol Tosylation and Displacement Followed By Suzuki Coupling and Ester Reduction

Example 2-29: 5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one

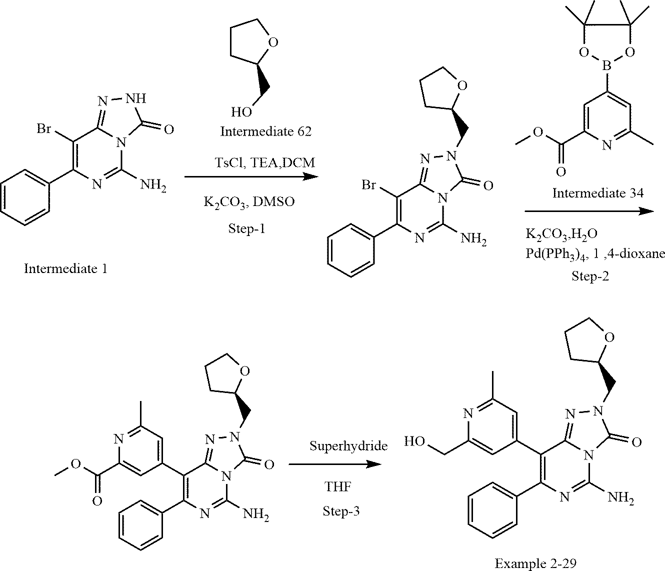

Step 1; To a solution of N,N-dimethylpyridin-4-amine (19.9 mg, 0.16 mmol), TEA (247.5 mg, 2.45 mmol) and tosyl chloride (342.0 mg, 1.79 mmol) in DCM (20 mL) at 0° C. was added (R)-(tetrahydrofuran-2-yl)methanol (199.9 mg, 1.96 mmol) and the reaction was stirred at room temperature for 1 h. The reaction mixture was partitioned between DCM (20 mL) and H₂O (20 mL). The organic layer was separated and concentrated under reduced pressure to obtain tosylated intermediate. The tosylated intermediate was taken in DMSO (30 mL) and 5-amino-8-bromo-7-phenyl-[1,2,4]triazolo[4,3-c]pyrimidin-3(2H)-one (500 mg, 1.63 mmol) and K₂CO₃ (676 mg, 4.09 mmol) were added, then heated to 80° C. for 4 h. The reaction mixture was partitioned between EtOAc (20 mL) and H₂O (20 mL). The organic layer was separated and concentrated under reduced pressure to afford (R)-5-amino-8-bromo-7-phenyl-2-((tetrahydrofuran-2-yl)methyl)-[1,2,4]triazolo[4,3-c]pyrimidin-3(2H)-one (400 mg, 62%) as off white solid. The crude product was used in the next step without further purification.

LCMS (Method A): m/z 390 (M+H)⁺ (ES⁺), at 2.38 min, UV active.

¹H NMR: (400 MHz, DMSO-d6) δ: 7.82 - 7.77 (m, 1H), 7.64 - 7.60 (m, 2H), 7.50 - 7.44 (m, 2H), 4.23 - 4.02 (m, 1H), 4.00 - 3.96 (m, 2H), 3.86 - 3.79 (m, 2H), 3.74 - 3.66 (m, 2H), 1.99 -1.91 (m, 2H). Exchangeable —NH₂ protons were not observed.

Step 2; To a suspension of (R)-5-amino-8-bromo-7-phenyl-2-((tetrahydrofuran-2-yl)methyl)-[1,2,4]triazolo[4,3-c]pyrimidin-3(2H)-one (200 mg, 0.51 mmol), methyl 6-methyl-4-(4,4,5,5-tetramethyl-1,3,2-dioxaborolan-2-yl)picolinate (170 mg, 0.61 mmol) and K₂CO₃ (212 mg, 1.53 mmol) in 1,4-dioxane (10 mL) and H₂O (5 mL) was added Pd(PPh₃)₄ (59 mg, 0.051 mmol) and heated to 110° C. for 5 h. The reaction mixture was partitioned between H₂O (20 mL) and EtOAc (30 mL). The organic layer was separated, dried over anhydrous Na₂SO₄ and concentrated under reduced pressure. The crude product was purified by Biotage-Isolera using 10 g silica snap and eluted with 0-100% EtOAc in Pet-ether gradient to afford methyl (R)-4-(5-amino-3-oxo-7-phenyl-2-((tetrahydrofuran-2-yl)methyl)-2,3-dihydro-[1,2,4]triazolo[4,3-c]pyrimidin-8-yl)-6-methylpicolinate (100 mg, 42%).

LCMS (Method B): m/z 461 (M+H)⁺ (ES⁺), at 2.15 min, UV active.

¹H NMR: (400 MHz, DMSO-d6) δ: 7.70 (s, 1H), 7.65 - 7.57 (m, 3H), 7.32 - 7.26 (m, 5H), 4.16 - 4.13 (m, 1H), 3.86 - 3.74 (m, 6H), 3.66 - 3.62 (m, 1H), 2.34 (s, 3 H), 2.00 - 1.67 (m, 3H), 1.25 - 1.18 (m, 1H).

Step 3; To a solution of methyl (R)-4-(5-amino-3-oxo-7-phenyl-2-((tetrahydrofuran-2-yl)methyl)-2,3-dihydro-[1,2,4]triazolo[4,3-c]pyrimidin-8-yl)-6-methylpicolinate (100 mg, 0.21 mmol) in THF at room temperature was added lithium triethyl borohydride (34.5 mg, 0.32 mmol) portion wise and stirred for 1 h. The reaction mixture was partitioned between ethyl acetate (20 mL) and H₂O (10 mL). The organic layer was separated, dried over anhydrous Na₂SO₄ and concentrated under reduced pressure. The crude product was purified by Prep-HPLC (Method A), fractions were concentrated and the residue was diluted with ethyl acetate (10 mL), washed with 10% sodium bicarbonate solution (10 mL). The organic layer was separated, dried over anhydrous Na₂SO₄ and concentrated to dryness to afford 5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one (23 mg, 24%) as a yellow solid.

Example 2-29 was further purified by SFC Method A. During purification, the enantiomer, Example 2-28 (5-amino-8-[2-(hydroxymethyl)-6-methyl-4-pyridyl]-7-phenyl-2-[[(2S)-tetrahydrofuran-2-yl]methyl]-[1,2,4]triazolo[4,3-c]pyrimidin-3-one) was isolated, presumably formed during the synthesis of Example 2-29, from a small quantity of the enantiomer of intermediate 62 present in the commercial sample. Using SFC Method A, the first eluting peak was Example 2-28, (9.73 min), and the second eluting peak was Example 2-29 (10.32 min).

### Synthetic Route R: Typical Procedure For the Preparation of Hydroxymethyl Pyridine Analogues Via Alkylation Followed By Suzuki And Ester Reduction

Example 1-42: 5-amino-8-[2-(hydroxymethyl)-6-methyl-4-pyridyl]-2-(oxazol-4-ylmethyl)-7-phenyl-[1,2,4]triazolo[4,3-c]pyrimidin-3-one

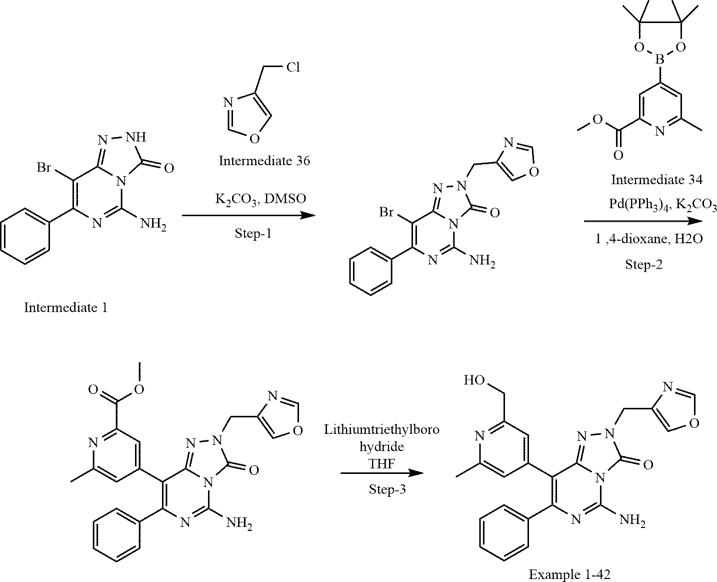

Step 1: Performed in a similar fashion to Route h, using Intermediate 36 to afford 5-amino-8-bromo-2-(oxazol-4-ylmethyl)-7-phenyl-[1,2,4]triazolo[4,3-c]pyrimidin-3(2H)-one as a white solid.

LCMS (Method C): m/z 388 (M+H)⁺ (ES⁺), at 1.82 min, UV active.

¹H NMR: (400 MHz, DMSO-d6) δ: 8.37 (s, 1H), 8.14 (s, 1H), 7.60 (d, J = 6.4 Hz, 2H), 7.49 - 7.39 (m, 3H), 4.96 (s, 2H). Exchangeable —NH₂ Protons were not observed.

Step 2: Performed in a similar fashion to Route a step 2, using Intermediate 34 to afford methyl 4-(5-amino-2-(oxazol-4-ylmethyl)-3-oxo-7-phenyl-2,3-dihydro-[1,2,4]triazolo[4,3-c]pyrimidin-8-yl)-6-methylpicolinate as a yellow solid.

LCMS (Method C): m/z 458 (M+H)⁺ (ES⁺), at 1.58 min, UV active.

¹H NMR: (400 MHz, DMSO-d6) δ: 8.36 (s, 1H), 8.09 (s, 1H), 7.67 (s, 1H), 7.31-7.23 (m, 6H), 4.94 (s, 2H), 3.80 (s, 3H), 2.28 (s, 3H). Exchangeable —NH₂ protons were not observed.

Step 3: Performed in a similar fashion to Route o, step 2 to afford 5-amino-8-[2-(hydroxymethyl)-6-methyl-4-pyridyl]-2-(oxazol-4-ylmethyl)-7-phenyl-[1,2,4]triazolo[4,3-c]pyrimidin-3-one as an off white solid. The data for the title compound are in Table 2.

### Synthetic Route S: Typical Procedure For The Preparation of Hydroxymethyl Pyridine Analogues Via Mitsunobu Reaction Followed By Ester Reduction

Example 1-43: 5-amino-8-[2-(hydroxymethyl)-6-methyl-4-pyridyl]-2-[(5-methylisoxazol-3-yl)methyl]-7-phenyl-[1,2,4]triazolo[4,3-c]pyrimidin-3-one

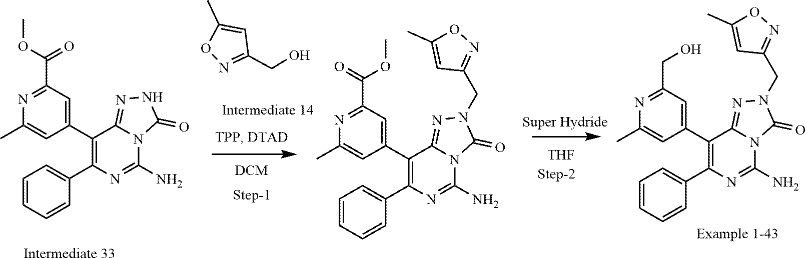

Step 1: Prepared in a similar fashion to route d, using intermediate 14 and DCM as solvent, then purified by Biotage-Isolera using 10 g silica snap and was eluted with 0-100% EtOAc in pet ether gradient to afford methyl 4-(5-amino-2-((5-methylisoxazol-3-yl)methyl)-3-oxo-7-phenyl-2,3-dihydro-[1,2,4]triazolo[4,3-c]pyrimidin-8-yl)-6-methylpicolinate as a pale yellow solid.

LCMS (Method C): m/z 472 (M+H)⁺ (ES⁺), at 1.82 min, UV active.

¹H NMR: (400 MHz, DMSO-d6) δ: 7.69 (s, 1H), 7.32-7.23 (m, 6H), 6.22 (s, 1H), 5.06 (s, 2H), 3.81 (s, 3H), 2.34 (s, 6H). Exchangeable —NH₂ protons were not observed.

Step 2: Performed in a similar fashion to Route o, step 2 to afford 5-amino-8-(2-(hydroxymethyl)-6-methylpyridin-4-yl)-2-((5-methylisoxazol-3-yl)methyl)-7-phenyl-[1,2,4]triazolo[4,3-c]pyrimidin-3(2H)-one as a yellow solid. The data for the title compound are in Table 2.

### Synthetic Route T: Typical Procedure For The Preparation of Hydroxymethyl Pyridine Analogues Via An Alkylation Reaction Followed by Ester Reduction

Example 1-46: 5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one

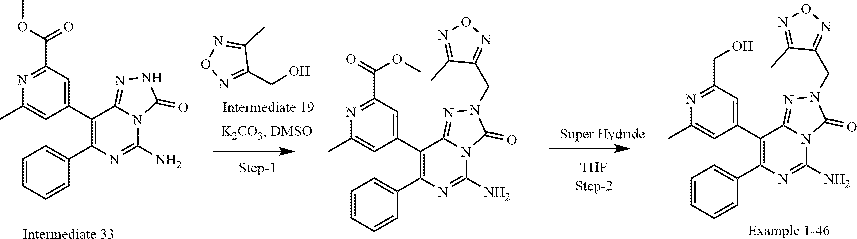

Step 1: Prepared in a similar fashion to route h, to afford methyl 4-(5-amino-2-((4-methyl-1,2,5-oxadiazol-3-yl)methyl)-3-oxo-7-phenyl-2,3-dihydro-[1,2,4]triazolo[4,3-c]pyrimidin-8-yl)-6-methylpicolinate as a yellow solid.

LCMS (Method B): m/z 473 (M+H)⁺ (ES⁺), at 1.91 min, UV active.

¹H NMR: (400 MHz, DMSO-d6) δ: 7.68 (s, 1H), 7.30 - 7.24 (m, 5H), 7.18 (s, 1H), 5.30 (s, 2H), 3.81 (s, 3H), 2.46 (s, 3H), 2.41 (s, 3H). Exchangeable —NH₂ protons were not observed.

Step 2: Performed in a similar fashion to Route o, step 2 to afford 5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one as a yellow solid. The data for the title compound are in Table 2.

### Synthetic Route U

Example 1-53: 5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one

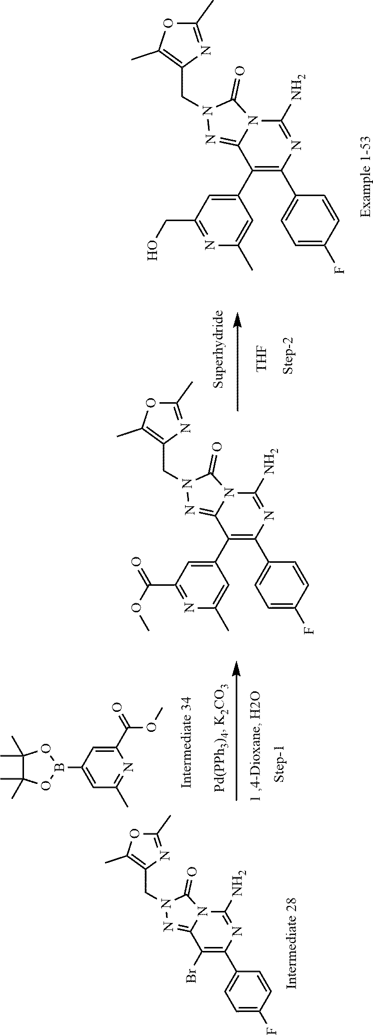

Step 1: Prepared in a similar fashion to route a, step 2, using intermediate 34, to afford methyl 4-(5-amino-2-((2,5-dimethyloxazol-4-yl)methyl)-7-(4-fluorophenyl)-3-oxo-2,3-dihydro-[1,2,4]triazolo[4,3-c]pyrimidin-8-yl)-6-methylpicolinate (1.6 g, 55%) as a yellow solid.

LCMS (Method C): m/z 504 (M+H)⁺ (ES⁺), at 1.76 min, UV active.

¹H NMR: (400 MHz, DMSO-d6) δ: 7.68 (s, 1H), 7.32-7.28 (m, 2H), 7.23 (s, 1H), 7.14-7.09 (m, 2H), 5.76 (s, 2H), 3.82 (s, 3H), 2.40 (s, 9H). Exchangeable —NH2 protons were not observed.

Step 2: Performed in a similar fashion to Route o, step 2 to afford 5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one (270 mg, 29%) as a yellow solid. The data for the title compound are in Table 2.

### Synthetic Route V: Typical Procedure For The Preparation of Hydroxymethyl Pyridine Analogues Via Alcohol Chlorination And Displacement, Followed by Suzuki Coupling and Ester Reduction

Example 1-55: 5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one

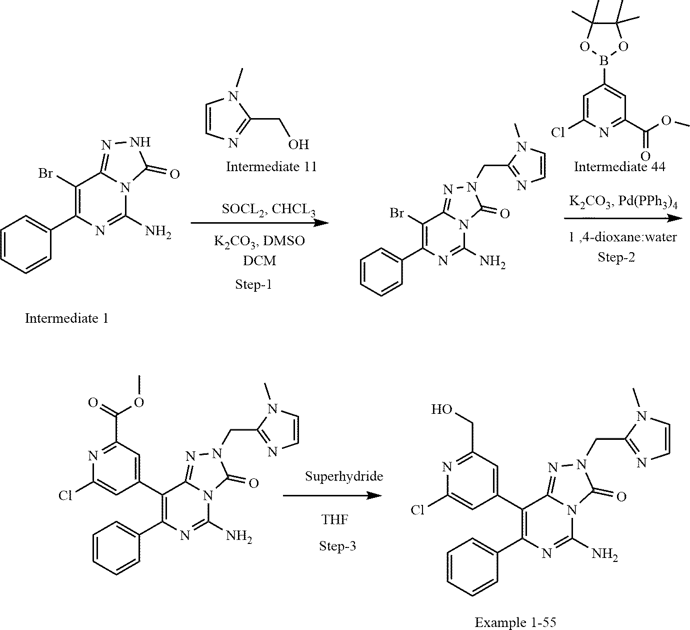

Step 1: To a solution of (1-methyl-1H-imidazol-2-yl)methanol (220 mg, 1.960 mmol) in CHCl₃, SOCl₂ (291 mg, 2.45 mmol) was added at 0° C. and the resultant reaction mixture was stirred at 50° C. for 2 h. After the completion of starting material, monitored by TLC, reaction mixture was concentrated under reduced pressure to afford the chlorinated intermediate. The chlorinated intermediate was taken in DMSO (20 mL), 5-amino-8-bromo-7-phenyl-[1,2,4]triazolo[4,3-c]pyrimidin-3(2H)-one (500 mg, 1.633 mmol) and K₂CO₃ (676 mg, 4.901 mmol) were added and the reaction mixture was heated to 60° C. for 2 h. After the completion of starting material, monitored by TLC, the reaction mixture was poured into ice water to obtain solid and the obtained solid was filtered through Buchner funnel and dried under vacuum to afford 5-amino-8-bromo-2-((1-methyl-1H-imidazol-2-yl)methyl)-7-phenyl-[1,2,4]triazolo[4,3-c]pyrimidin-3(2H)-one (600 mg, 87%) as yellow solid.

LCMS (Method C): m/z 400 (M+H)⁺ (ES⁺), at 2.27 min, UV active.

¹H NMR: (400 MHz, DMSO-d6) δ 7.62-7.60 (m, 2H), 7.45 (d, J = 6.4 Hz, 3H), 7.15 (s, 1H), 6.82 (s, 1H), 5.12 (s, 2H), 3.72 (s, 3H). Exchangeable —NH₂ protons were not observed.

(Alternatively, for some analogues, the reaction mixture was partitioned between EtOAc (30 mL) and water (3 × 20 mL). The organic layer was separated and concentrated under reduced pressure. The crude was purified by flash column chromatography by using silica mesh (230-400) and was eluted with 0-100% EtOAc in pet ether gradient to afford the desired product)

Step 2: Prepared in a similar fashion to route a, step 2, using intermediate 44, to afford methyl 4-(5-amino-2-((1-methyl-1H-imidazol-2-yl)methyl)-3-oxo-7-phenyl-2,3-dihydro-[1,2,4]triazolo[4,3-c]pyrimidin-8-yl)-6-chloropicolinate (100 mg, 26%) as yellow solid.

LCMS (Method C): m/z 491 (M+H)⁺ (ES⁺), at 1.29 min, UV active.

Step 3: Performed in a similar fashion to Route o, step 2 to afford 5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one (13 mg, 14%) as a yellow solid. The data for the title compound are in Table 2.

### Synthetic Route W: Typical Procedure For The Boc-Deprotection of Amine Analogues

Example 2-2: 5-amino-8-(2,6-dimethyl-4-pyridyl)-7-phenyl-2-pyrrolidin-3-yl-[1,2,4]triazolo[4,3-c]pyrimidin-3-one

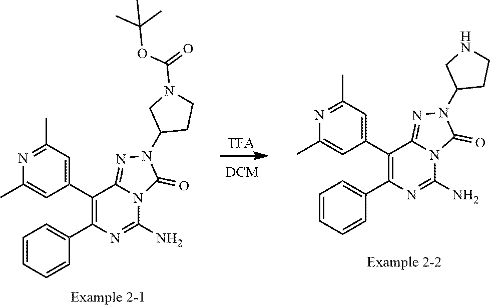

A solution of tert-butyl 3-(5-amino-8-(2,6-dimethylpyridin-4-yl)6-3-oxo-7-phenyl-[1,2,4]triazolo[4,3-c]pyrimidin-2(3H)-yl)pyrrolidine-1-carboxylate (120 mg, 0.23 mmol) in 20% TFA in DCM (5 mL) was stirred at room temperature for 15 h. After the completion of starting material confirmed by TLC, reaction mass was concentrated under reduced pressure. The crude mass was dissolved in MeOH (2 mL) and was passed through a DSC-SCX column (6 mL), washed with water (5 mL). The compound was eluted with 2 M ammonia in MeOH (10 mL), concentrated and lyophilized to afford 5-amino-8-(2,6-dimethyl-4-pyridyl)-7-phenyl-2-pyrrolidin-3-yl-[1,2,4]triazolo[4,3-c]pyrimidin-3-one (26 mg, 27%) as a yellow solid. The data for the title compound are in Table 2.

### Synthetic Route X: Typical Procedure For The Reductive Amination of Amines Using Formaldehyde

Example 2-6: 5-amino-8-(2,6-dimethyl-4-pyridyl)-2-[(1-methylpyrrolidin-2-yl)methyl]-7-phenyl-[1,2,4]triazolo[4,3-c]pyrimidin-3-one

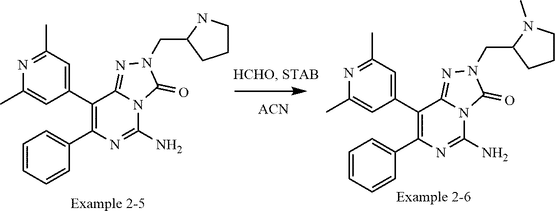

To a suspension of 5-amino-8-(2,6-dimethylpyridin-4-yl)-7-phenyl-2-(pyrrolidin-2-ylmethyl)-[1,2,4]triazolo[4,3-c]pyrimidin-3(2H)-one (90 mg, 0.21 mmol) in MeCN (10 mL) was added 36% formaldehyde solution (0.02 mL, 0.23 mmol) and sodium triacetoxy borohydride (133 mg, 0.63 mmol) and stirred for 1 h at room temperature. The reaction was quenched with water (5 mL) and extracted with EtOAc (2 × 10 mL). The combined organic layers were dried over anhydrous sodium sulphate and concentrated under reduced pressure to afford 5-amino-8-(2,6-dimethyl-4-pyridyl)-2-[(1-methylpyrrolidin-2-yl)methyl]-7-phenyl-[1,2,4]triazolo[4,3-c]pyrimidin-3-one (75 mg, 80%) as a yellow solid. The data for the title compound are in Table 2.

### Synthetic Route Y: Typical Procedure for the Preparation of Di-Fluorinated Pyrrolidine Analogues

Example 2-11: 5-amino-2-[[(2S)-4,4-difhioropyrrolidin-2-yl]methyl]-8-(2,6-dimethyl-4-pyridyl)-7-phenyl-[1,2,4]triazolo[4,3-c]pyrimidin-3-one

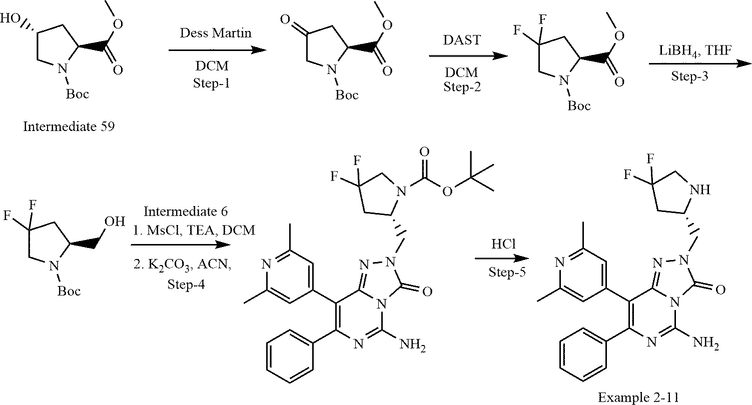

Step 1: To a suspension of 1-(tert-butyl) 2-methyl (2S,4R)-4-hydroxypyrrolidine-1,2-dicarboxylate (2 g, 8.22 mmol) in DCM (30 mL) at 0° C. was added Dess-Martin Periodinane (7 g, 16.44 mmol) and stirred at room temperature for 3 h. The reaction was partitioned between EtOAc (100 mL) and saturated NaHCO₃ solution (10 mL). The organic layer was separated, dried over anhydrous Na₂SO₄ and concentrated under reduced pressure. The crude compound was purified by Biotage-Isolera using 25 g silica snap and was eluted with gradient 0-20% EtOAc in Hexane to afford 1-(tert-butyl) 2-methyl (S)-4-oxopyrrolidine-1,2-dicarboxylate (1.6 g, 74%) as a colourless gum.

¹H NMR: (400 MHz, DMSO-d6) δ: 4.67 - 4.62 (m, 1H), 3.88 - 3.80 (m, 1H), 3.72 - 3.66 (m, 4H), 3.15 - 3.07 (m, 1H), 2.63 - 2.55 (m, 1H), 1.40 (d, J = 16.0 Hz, 9H).

Step 2: To a suspension of 1-(tert-butyl) 2-methyl (S)-4-oxopyrrolidine-1,2-dicarboxylate (1.5 g, 6.16 mmol) in DCM at -78° C. was added diethylaminosulfur trifluoride (1.98 g, 12.32 mmol) dropwise and stirred at room temperature for 15 h. The reaction was quenched with saturated NaHCO₃ solution (20 mL) and extracted with DCM (2 × 30 mL). The combined organic layers were dried over anhydrous Na₂SO₄ and concentrated under reduced pressure. The crude product was purified by Biotage-Isolera using 25 g silica snap and was eluted with gradient 0-10% EtOAc in Hexane to afford 1-(tert-butyl) 2-methyl (S)-4,4-difluoropyrrolidine-1,2-dicarboxylate (1.4 g, 94%) as a colourless gum.

¹H NMR: (400 MHz, DMSO-d6) δ: 4.58 - 4.45 (m, 1H), 3.91 - 3.77 (m, 5H), 2.76 - 2.67 (m, 1H), 2.54 - 2.44 (m, 1H), 1.44 (d, J = 18.0 Hz, 9H).

Step 3: To a solution of 1-(tert-butyl) 2-methyl (S)-4,4-difluoropyrrolidine-1,2-dicarboxylate (1.1 g, 4.15 mmol) in THF (20 mL) at 0° C. was added 2 M LiBH₄ solution (3.1 mL, 6.22 mmol) and stirred at room temperature for 2 h. The reaction was quenched by the dropwise addition of saturated NH₄Cl solution (25 mL) and extracted with EtOAc (30 mL). The organic layer was separated, dried over anhydrous Na₂SO₄ and concentrated under reduced pressure to afford tert-butyl (S)-4,4-difluoro-2-(hydroxymethyl)pyrrolidine-1-carboxylate (500 mg, 50%) as an off-white gum.

¹H NMR: (400 MHz, DMSO-d6) δ: 4.20 - 4.18 (m, 1H), 3.77 - 3.65 (m, 4H), 2.54 - 2.49 (m, 1H), 2.20 - 2.18 (m, 1H), 1.50 (s, 9H). Exchangeable —OH proton was not observed.

Step 4: Prepared in a similar fashion to route e, purified by Biotage-Isolera using 25 g silica snap and was eluted with gradient 0-100% EtOAc in hexane to afford tert-butyl (S)-2-((5-amino-8-(2,6-dimethylpyridin-4-yl)-3-oxo-7-phenyl-[1,2,4]triazolo[4,3-c]pyrimidin-2(3H)-yl)methyl)-4,4-difluoropyrrolidine-1-carboxylate (150 mg, 18%) as a yellow solid.

LCMS (Method A): m/z 552 (M+H)⁺ (ES⁺), at 3.68 min, UV active.

¹H NMR: (400 MHz, DMSO-d6) δ: 8.30 - 7.70 (m, 2H), 7.27 - 7.25 (m, 5H), 6.84 (s, 2H), 4.31 - 4.29 (m, 1H), 4.05 - 4.03 (m, 2H), 3.98 - 3.96 (m, 1H), 3.94 - 3.92 (m, 1H), 2.70 -2.60 (m, 2H), 2.22 (s, 6H), 1.34 (s, 9H).

Step 5: To a solution of tert-butyl (S)-2-((5-amino-8-(2,6-dimethylpyridin-4-yl)-3-oxo-7-phenyl-[1,2,4]triazolo[4,3-c]pyrimidin-2(3H)-yl)methyl)-4,4-difluoropyrrolidine-1-carboxylate (220 mg, 0.39 mmol) in 1,4-Dioxane (3 mL) at room temperature was added 4 N HCl in 1,4-Dioxane (3 mL) and the reaction mixture was stirred at room temperature for 5 h. The reaction mixture was concentrated under reduced pressure then partitioned between EtOAc (10 mL) and saturated NaHCO₃ solution (10 mL). The organic layer was separated, dried over anhydrous Na₂SO₄ and concentrated under reduced pressure to afford 5-amino-2-[[(2S)-4,4-difluoropyrrolidin-2-yl]methyl]-8-(2,6-dimethyl-4-pyridyl)-7-phenyl-[1,2,4]triazolo[4,3-c]pyrimidin-3-one (100 mg, 81%) as a yellow solid. The data for the title compound are in Table 2.

### Synthetic Route Z: Typical Procedure For The Preparation of Alkylated Triazolopyrimidinones Utilising a Boc-Protection Strategy

Example 2-15: 5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one

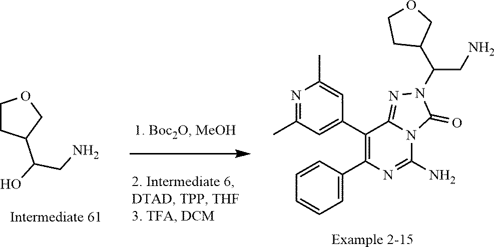

2-amino-1-(oxolan-3-yl)ethan-1-ol (131 mg, 1 mmol) was dissolved in MeOH (2 mL), the solution was cooled to 0° C. and Boc₂O (229 mg; 1.05 mmol) was added in one portion. The reaction mixture stirred at room temperature for 2-3 h. The mixture was concentrated under reduced pressure and the residue was crystallized from the mixture of i-Pr/hexane to give the Boc-protected amine. To a solution of 5-amino-8-(2,6-dimethylpyridin-4-yl)-7-phenyl-[1,2,4]triazolo[4,3-c]pyrimidin-3(2H)-one (66 mg; 0.20 mmol), Boc protected amine (28 mg, 0.22 mmol) and PPh3 (68 mg, 0.26 mmol) in THF (3 mL), was added di-t-Butyl-azodicarboxylate (51 mg, 0.22 mmol). The reaction mixture was stirred at room temperature for 16-18 h. The mixture was concentrated under reduced pressure, the residue was dissolved in DCM (3 mL) and TFA (0.5 mL) was added. The reaction mixture was sonicated at room temperature for 2 h, then concentrated under reduced pressure and the crude product was purified by prep HPLC-MS (C18 Column 100x19 mm, FLO/MeOH or MeCN) to afford 5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one (36 mg, 41%). The data for the title compound are in Table 2.

### Synthetic Route Aa: Typical Procedure for the Preparation of Fluorinated Pyrrolidine Analogues

Example 2-18: 5-amino-8-(2,6-dimethyl-4-pyridyl)-2-[[(2R,4S)-4-fluoropyrrolidin-2-yl]methyl]-7-phenyl-[1,2,4]triazolo[4,3-c]pyrimidin-3-one

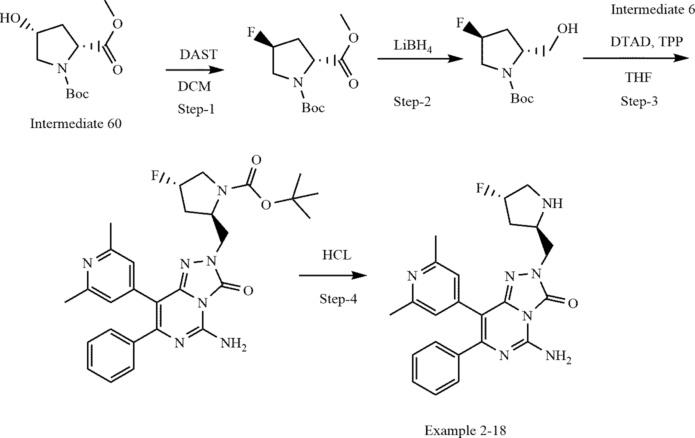

Step 1: To a suspension of 1-(tert-butyl) 2-methyl (2R,4R)-4-hydroxypyrrolidine-1,2-dicarboxylate (0.5 g, 2.038 mmol) in DCM at -78° C. was added diethylaminosulfur trifluoride (0.53 mL, 4.076 mmol) dropwise and stirred at room temperature for 15 h. The reaction mass was quenched with saturated NaHCO₃ solution (10 mL) and extracted with DCM (20 mL). The combined organic layers were dried over anhydrous Na₂SO₄ and concentrated under reduced pressure. The crude product was purified by Biotage-Isolera using 10 g silica snap and was eluted with gradient 0-10% EtOAc in Hexane to afford 1-(tert-butyl) 2-methyl (2R,4S)-4-fluoropyrrolidine-1,2-dicarboxylate (0.23 g, 45%) as a colourless gum.

¹H NMR: (400 MHz, CDCl₃) δ: 5.30 - 5.17 (m, 1H), 4.51 - 4.40 (m, 1H), 3.98 - 3.82 (m, 1H), 3.78 (s, 3H), 3.71 - 3.51 (m, 1H), 2.67 - 2.56 (m, 1H), 2.21 - 2.04 (m, 1H), 1.44 (s, 9H).

Step 2: Prepared in a similar fashion to route y step-3, to afford tert-butyl (2R, 4S)-4-fluoro-2-(hydroxymethyl) pyrrolidine-1-carboxylate (180 mg, 88%) as a colourless gum.

¹H NMR: (400 MHz, CDCl₃) δ: 5.19 - 5.06 (m, 1H), 4.79 (s, 2H), 4.17 - 3.79 (m, 3H), 3.62 - 3.50 (m, 1H), 3.43 - 3.41 (m, 1H), 2.42 - 2.32 (m, 1H), 1.50 (s, 9H).

Step 3: Prepared in a similar fashion to route d, purified by Biotage-Isolera using 10 g silica snap and was eluted with gradient 0-50% EtOAc in Hexane to afford tert-butyl (2R,4S)-2-((5-amino-8-(2,6-dimethylpyridin-4-yl)-3-oxo-7-phenyl-[1,2,4]triazolo[4,3-c]pyrimidin-2(3H)-yl)methyl)-4-fluoropyrrolidine-1-carboxylate (60 mg, 14%) as a yellow solid.

LCMS (Method A): m/z 533 (M+H)⁺ (ES⁺), at 3.40 min, UV active.

¹H NMR: (400 MHz, DMSO-d6) δ: 7.28 - 7.23 (m, 5H), 6.81 (s, 2H), 5.28 - 5.10 (m, 1H), 4.18 - 4.10 (m, 1H), 3.95 - 3.92 (m, 2H), 2.33 - 2.26 (m, 8H), 2.17 - 1.99 (m, 2H), 1.15 (s, 9H). Exchangeable –NH₂ protons were not observed.

Step 4: Prepared in a similar fashion to route y step 5, to afford 5-amino-8–(2,6–dimethyl-4–pyridyl)-2–[[(2R,4S)-4–fluoropyrrolidin-2–yl]methyl]-7-phenyl-[1,2,4]triazolo[4,3–c]pyrimidin-3-one (20 mg, 36%) as a yellow solid. The data for the title compound are in Table 2.

### Synthetic Route Ab: Typical Procedure for the Preparation of Alkylated Triazolopyrimidinones Utilising a MOM-Protection Strategy

Example 2–22: 5-amino-8-(2,6-dimethyl-4-pyridyl)-2-[[2S,4S)-4-hydroxypyrrolidin-2-yl]methyl]-7-phenyl-[1,2,4]triazolo[4,3–c]pyramidin-3–one

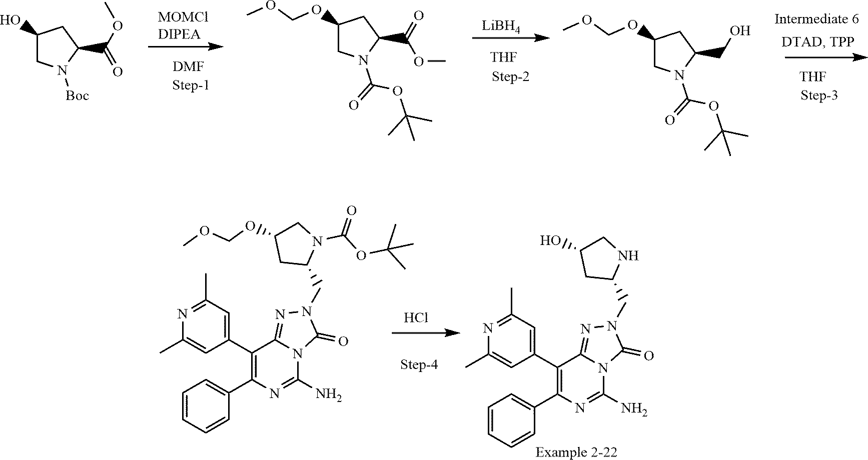

Step 1: To a suspension of 1-(tert-butyl) 2-methyl (2S,4S)-4-hydroxypyrrolidine-1,2-dicarboxylate (0.5 g, 2.0 mmol) in (50 mL) DMF was added di-isopropyl ethylamine (1.3 g, 1.0 mmol) at room temperature and was cooled to 0° C. and added MOM chloride (0.66 g, 8.15 mmol) dropwise. The reaction mixture was stirred at room temperature for 12 h. The reaction mass was quenched by adding water (20 mL) and extracted with EtOAc (2 × 20 mL). The combined organic layers were dried over anhydrous Na₂SO₄ and concentrated under reduced pressure. The crude compound was purified by Biotage-Isolera using 10 g silica snap and was eluted with gradient 0-50% EtOAc in Hexane to afford 1-(tert-butyl) 2-methyl (2S,4S)-4-(methoxymethoxy)pyrrolidine-1,2-dicarboxylate (0.4 g, 68%) as a yellow liquid.

¹H NMR: (400 MHz, DMSO-d6) δ: 4.56 (m, 2H), 4.29 - 4.24 (m, 2H), 3.58 - 3.58 (m, 4H), 3.23 (s, 4H), 3.23 (s, 1H), 2.01 - 1.99 (m, 1H), 1.20 (s, 9H).

Step 2: Prepared in a similar fashion to route y step-3, purified by Biotage-Isolera using 10 g silica snap and eluted with gradient 0-50% EtOAc in Hexane to afford tert-butyl (2S,4S)-2-(hydroxymethyl)-4-(methoxymethoxy)pyrrolidine-1-carboxylate (0.3 g, 83%) as a colourless liquid.

¹H NMR: (400 MHz, DMSO-d6) δ: 4.68 - 4.62 (m, 1H), 4.60 - 4.56 (m, 2H), 4.15 - 4.05 (m, 1H), 3.69 - 3.57 (m, 4H), 3.21 (s, 4H), 2.00 - 1.99 (m, 2H), 1.40 (s, 9H).

Step 3: Prepared in a similar fashion to route d, purified by Biotage-Isolera using 10 g silica snap and eluted with gradient 0-100% EtOAc in Hexane to afford tert-butyl (2S,4S)-2-((5-amino-8-(2,6-dimethylpyridin-4-yl)-3-oxo-7-phenyl-[1,2,4]triazolo[4,3-c]pyrimidin-2(3H)-yl)methyl)-4-(methoxymethoxy) pyrrolidine-1-carboxylate (130 mg, 30%) as yellow solid.

LCMS (Method B): m/z 576 (M+H)⁺ (ES⁺), at 2.29 min, UV active.

¹H NMR: (400 MHz, DMSO-d6) δ: 7.28 - 7.25 (m, 5H), 6.82 (s, 2H), 4.64 - 4.63 (m, 1H), 4.58 - 4.42 (m, 1H), 4.30 - 4.25 (m, 1H), 4.11 - 4.09 (m, 2H), 4.07 - 4.06 (m, 1H), 3.95 -3.93 (m, 1H), 3.24 (s, 3H), 2.28 (s, 6H), 2.11 - 2.06 (m, 3H), 1.33 (s, 9H). Exchangeable -NH₂ protons were not observed.

Step 4: Prepared in a similar fashion to route y, step-5 to afford 5-amino-8-(2,6-dimethyl-4-pyridyl)-2-[[(2S,4S)-4-hydroxypyrrolidin-2-yl]methyl]-7-phenyl-[1,2,4]triazolo[4,3-c]pyrimidin-3-one (70 mg, 71%) as a yellow solid. The data for the title compound are in Table 2.

### Synthetic Route Ac

Example 2-27: 5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3-one

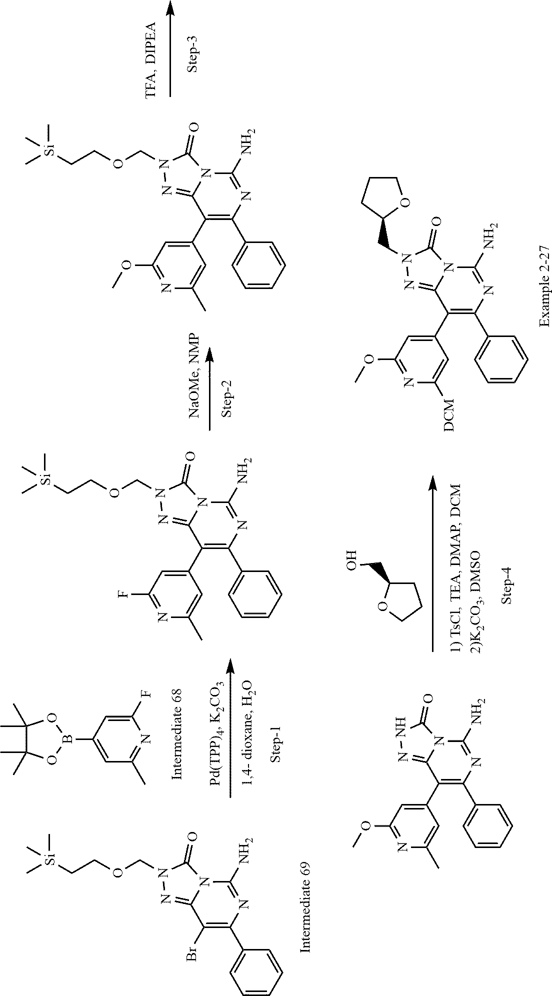

Step 1: Prepared in a similar fashion to route a, step 2 to afford 5-amino-8-(2-fluoro-6-methylpyridin-4-yl)-7-phenyl-2-((2-(trimethylsilyl)ethoxy)methyl)-[1,2,4]triazolo[4,3-c]pyrimidin-3(2H)-one (1.4 g, 26%) as a yellow solid.

LCMS (Method C): m/z 467 (M+H)⁺ (ES⁺), at 2.60 min, UV active.

Step 2: To a solution of 5-amino-8-(2-fluoro-6-methylpyridin-4-yl)-7-phenyl-2-((2-(trimethylsilyl)ethoxy)methyl)-[1,2,4]triazolo[4,3-c]pyrimidin-3(2H)-one (1.4 g, 2.99 mmol) in MeOH, NaOMe in 25% MeOH (1.3 mL, 6.0 mmol) was added in a sealed tube and the resultant reaction mixture was heated to 90° C. for 16 h. After the completion of starting material, monitored by TLC, reaction mixture was partitioned between H₂O (25 mL) and EtOAc (50 mL). Organic layer was separated, dried over anhydrous Na₂SO₄ and concentrated to get crude mass. The crude was purified by Biotage-Isolera using 25 g silica gel snap and was eluted with 0-80% EtOAc in Pet ether gradient to afford 5-amino-8-(2-methoxy-6-methylpyridin-4-yl)-7-phenyl-2-((2-(trimethylsilyl)ethoxy)methyl)-[1,2,4]triazolo[4,3-c]pyrimidin-3(2H)-one (600 mg, 43%) as a yellow solid.

LCMS (Method C): m/z 479 (M+H)⁺ (ES⁺), at 2.86 min, UV active.

¹H NMR: (400 MHz, DMSO-d6) δ: 7.28-7.24 (m, 5H), 6.57 (s, 1H), 6.41 (s, 1H), 5.12 (s, 2H), 3.93 (s, 2H), 3.75 (s, 3H), 3.32 (d, J = 8.4 Hz, 2H), 2.24 (s, 3H), 1.07-1.04 (m, 9H). Exchangeable - NH₂ Protons were not observed.

Step 3: Prepared in a similar fashion to route 6, step 2 to afford 5-amino-8-(2-methoxy-6-methylpyridin-4-yl)-7-phenyl-[1,2,4]triazolo[4,3-c]pyrimidin-3(2H)-one (250 mg, 57%) as a yellow solid.

LCMS (Method C): m/z 349 (M+H)⁺ (ES⁺), at 1.51 min, UV active.

¹H NMR: (400 MHz, DMSO-d6) δ: 7.28 - 7.22 (m, 5H), 6.63 (s, 1H), 6.34 (s, 1H), 3.75 (s, 3H), 2.26 (s, 3H). Exchangeable —NH and —NH₂ Protons were not observed.

Step 4: Prepared in a similar fashion to route f, using intermediate 62, to afford 5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3-one (7 mg, 5%) as an off-white solid. The data for the title compound are in Table 2.

### Synthetic Route Ad: Typical Procedure for The Preparation of Alkylated Triazolopyrimidinones Amine Analogues Via a Mesylate Displacement

Example 3-1: 5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one

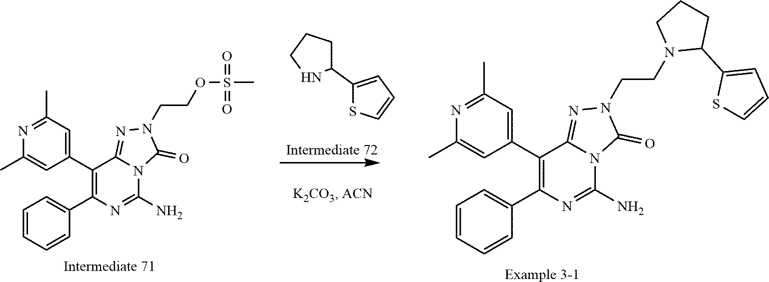

To a suspension 2-(5-amino-8-(2,6-dimethylpyridin-4-yl)-3-oxo-7-phenyl-[1,2,4]triazolo[4,3-c]pyrimidin-2(3H)-yl)ethyl methanesulfonate 1 (73 mg, 0.16 mmol) in MeCN (2 mL) was added K₂CO₃ (66 mg; 0.48 mmol) and 2-(thiophen-2-yl)pyrrolidine (29 mg, 0.19 mmol). The reaction mixture was heated at 100° C. for 15 h, then partitioned between EtOAc (5 mL) and H₂O (5 mL). The organic layer was separated, dried over anhydrous Na₂SO₄ and concentrated under reduced pressure. The crude product was purified by prep HPLC (method E) to afford 5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one (18 mg, 22%). The data for the title compound are in Table 2.

### Synthetic Route ae

Example 4-1: 5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one

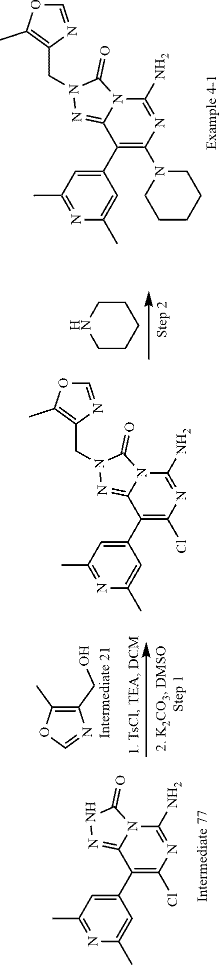

Step 1: Prepared in a similar fashion to route f, using intermediate 77, purified by Biotage-Isolera using 25 g silica gel snap and eluted with 0-80% EtOAc in Pet ether gradient to afford 5-amino-7-chloro-8-(2,6-dimethylpyridin-4-yl)-2-((5-methyloxazol-4-yl)methyl)-[1,2,4]triazolo[4,3-c]pyrimidin-3(2H)-one (560 mg, 40%) as an off white solid.

LCMS (Method C): m/z 386 (M+H)⁺ (ES⁺), at 0.98 min, UV active.

¹H NMR: (400 MHz, DMSO-d6) δ: 8.75 (s, 1H), 7.09 (s, 2H), 4.80 (s, 2H), 2.43 (s, 6H), 2.30 (s, 3H). Exchangeable —NH₂ protons were not observed.

Step 2: A suspension of 5-amino-7-chloro-8-(2, 6-dimethylpyridin-4-yl)-2-((5-methyloxazol-4-yl) methyl)-[1,2,4] triazolo [4, 3-c] pyrimidin-3(2H)-one (560 mg, 1.45 mmol) and piperidine (2 mL) were taken in a sealed tube and heated to 100° C. for 16 h. The reaction mixture was partitioned between EtOAc (30 mL) and H₂O (2 × 20 mL). The organic layer was separated, dried over anhydrous Na₂SO₄ and concentrated under reduced pressure. The crude compound was purified by prep-HPLC (Method A). Fractions were concentrated and the residue obtained was diluted with EtOAc (20 mL) and washed with 10% sodium bicarbonate solution (15 mL). The organic layer was separated, dried over anhydrous Na₂SO₄ and concentrated to afford 5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one (70 mg, 11%) as a yellow solid. The data for the title compound are in Table 2.

TABLE 2

Examples 1-1 to 4-1

| | | | |
|---|---|---|---|
| Ex. | Name | Details | Structure |
| 1-1 | 5-amino-8-(2,6-dimethyl-4-pyridyl)-7-phenyl-2-(2-pyrazol-1-ylethyl)-[1,2,4]triazolo[4,3-c]pyrimidin-3-one | Synthetic route a | 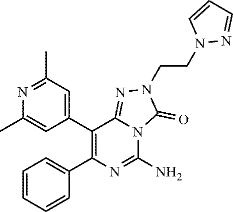 [SMILES: Cc1cc(-c2c(-c3ccccc3)nc(N)n3c(=O)n(CCn4cccn4)nc23)cc(C)n1] |
| | | ¹H NMR (400 MHz, DMSO-d6) δ 7.68 (d, J = 2.0 Hz, 1H), 7.44 (d, J = 1.2 Hz, 1H), 7.27 - 7.24 (m, 5H), 6.77 (s, 2H), 6.28-6.01 (m, 1H), 4.42 (t, J = 6.0 Hz, 2H), 4.14 (t, J = 6.0 Hz, 2H), 2.28 (s, 6H). | |
| | | Exchangeable —NH₂ protons were not observed | |
| | | LCMS Method A: m/z 427 (M+H)⁺ (ES+), at 2.46 min, UV active | |
| 1-2 | 5-amino-8-(2,6-dimethyl-4-pyridyl)-2-[2-(1-methylimidazol-2-yl)ethyl]-7-phenyl-[1,2,4]triazolo[4,3-c]pyrimidin-3-one | Synthetic route a; | 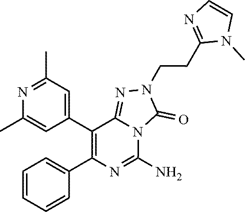 |
| | | Intermediates 1 & 4 (step-1) | |
| | | ¹H NMR (400 MHz, DMSO-d6) δ: 8.08 - 7.85 (m, 2H), 7.29 - 7.26 (m, 5H), 7.04 (s, 1H), 6.80 (s, 2H), 6.76 (s, 1H), 4.11 (t, J = 7.2 Hz, 2H), 3.55 (s, 3H), 3.03 (t, J = 7.2 Hz, 2H), 2.30 (s, 6H). | |
| | | LCMS Method A: m/z 441 (M+H)⁺ (ES+), at 1.90 min, UV active | |
| 1-3 | 5-amino-8-(2,6-dimethyl-4-pyridyl)-2-[2-(1H-imidazol-2-yl)ethyl]-7-phenyl-[1,2,4]triazolo[4,3-c]pyrimidin-3-one | Synthetic route a | 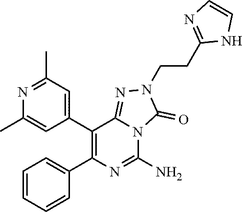 [SMILES: Cc1cc(-c2c(-c3ccccc3)nc(N)n3c(=O)n(CCc4ncc[nH]4)nc23)cc(C)n1] |
| | | Intermediates 1 & 5 (step-1) | |
| | | ¹H NMR (400 MHz, DMSO-d6) δ: 11.81 (s, 1H), 8.30 - 7.80 (m, 2H), 7.40 - 7.26 (m, 5H), 7.00 - 6.90 (m, 2H), 6.79 (s, 2H), 4.07 (t, J = 3.2 Hz, 2H), 3.01 (t, J = 3.2 Hz, 2H), 2.30 (s, 6H). | |
| | | LCMS Method A: m/z 427 (M+H)⁺ (ES+), at 1.88 min, UV active | |
| 1-4 | 5-amino-8-(2,6-dimethyl-4-pyridyl)-7-phenyl-2-[2-(1H-tetrazol-5-yl)ethyl]-[1,2,4]triazolo[4,3-c]pyrimidin-3-one | Synthetic route b | 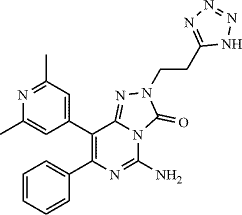 [SMILES: Cc1cc(-c2c(-c3ccccc3)nc(N)n3c(=O)n(CCc4nnn[nH]4)nc23)cc(C)n1] |
| | | ¹H NMR (400 MHz, DMSO-d6) δ: 8.71-8.35 (br. s, 2H), 7.26 - 7.25 (m, 5H), 6.73 (s, 2H), 4.14 (t, J = 6.6 Hz, 2H), 3.28 (t, J = 6.6 Hz, 2H), 2.29 (s, 6H). | |
| | | Exchangeable —NH proton was not observed | |
| | | LCMS Method A: m/z 429 (M+H)⁺ (ES+), at 2.08 min, UV active | |
| 1-5 | 5-amino-8-(2,6-dimethyl-4-pyridyl)-2-[2-(1-methyltetrazol-5-yl)ethyl]-7-phenyl-[1,2,4]triazolo[4,3-c]pyrimidin-3-one | Synthetic route c | 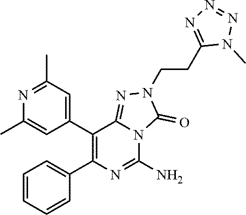 |
| | | ¹H NMR (400 MHz, DMSO-d6) δ: 7.60 - 7.51 (m, 1H), 7.46 - 7.43 (m, 4H), 7.35 - 7.25 (m, 2H), 4.58 (t, J = 6.0 Hz, 2H), 4.20 (s, 3H), 3.73 (t, J = 6.0 Hz, 2H), 2.68 (s, 6H). | |
| | | Exchangeable —NH₂ protons were not observed. | |
| | | LCMS Method A: m/z 443 (M+H)⁺ (ES+), at 2.17 min, UV active | |
| 1-6 | 5-amino-8-(2,6-dimethyl-4-pyridyl)-2-[2-(2-methyltetrazol-5-yl)ethyl]-7-phenyl-[1,2,4]triazolo[4,3-c]pyrimidin-3-one | Synthetic route c | 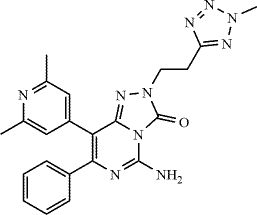 |
| | | ¹H NMR (400 MHz, DMSO-d6) δ: 8.30 (s, 2H), 7.70 - 7.66 (m, 5H), 6.78 (s, 2H), 4.30 (s, 3H), 4.15 (t, J = 6.1 Hz, 2H), 3.25 (t, J = 6.1 Hz, 2H), 2.30 (s, 6H). | |
| | | LCMS Method A: m/z 443 (M+H)⁺ (ES+), at 2.32 min, UV active | |
| 1-7 | 5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one | Synthetic route d | 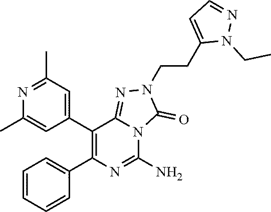 [SMILES: CCn1nccc1CCn1nc2c(-c3cc(C)nc(C)c3)c(-c3ccccc3)nc(N)n2c1=O] |
| | | ¹H NMR (400 MHz, Chloroform-d) δ: 7.43 (d, J = 1.9 Hz, 1H), 7.31 - 7.18 (m, 6H), 6.80 (s, 2H), 6.14 (d, J = 1.9 Hz, 1H), 4.27 - 4.03 (m, 4H), 3.14 (t, J = 7.2 Hz, 2H), 2.43 (s, 6H), 1.41 (t, J = 7.2 Hz, 3H). | |
| | | One exchangeable NH proton not observed. | |
| | | LCMS Method F: m/z 455 (M+H)⁺ (ES+), at 0.92 min, UV active | |
| 1-8 | 5-amino-8-(2,6-dimethyl-4-pyridyl)-7-phenyl-2-[1-(2-thienyl)ethyl]-[1,2,4]triazolo[4,3-c]pyrimidin-3-one | Synthetic route d; | 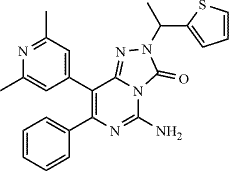 [SMILES: Cc1cc(-c2c(-c3ccccc3)nc(N)n3c(=O)n(C(C)c4cccs4)nc23)cc(C)n1] |
| | | Intermediates 6 & 8 | |
| | | ¹H NMR (400 MHz, DMSO-d6) δ 7.98 - 7.83 (m, 1H), 7.71 - 7.19 (m, 6H), 7.16 - 6.92 (m, 2H), 5.92 - 5.66 (m, 1H), 2.52 (s, 6H), 2.11 - 2.03 (m, 1H), 1.78 - 1.71 (m, 3H). | |
| | | Exchangeable —NH₂ Protons were not observed. | |
| | | LCMS Method F: m/z 443 (M+H)⁺ (ES+), at 1.09 min, UV active | |
| 1-9 | 5-amino-8-(2,6-dimethyl-4-pyridyl)-2-(oxazol-2-ylmethyl)-7-phenyl-[1,2,4]triazolo[4,3-c]pyrimidin-3-one | Synthetic route e | 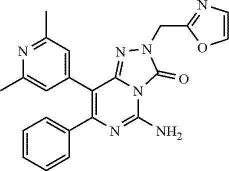 [SMILES: Cc1cc(-c2c(-c3ccccc3)nc(N)n3c(=O)n(Cc4ncco4)nc23)cc(C)n1] |
| | | ¹H NMR (400 MHz, DMSO-d6) δ: 8.12 (s, 1H), 7.27 (s, 5H), 7.20 (s, 1H), 6.80 (s, 2H), 5.21 (s, 2H), 2.28 (s, 6H). | |
| | | Exchangeable —NH₂ Protons were not observed. | |
| | | LCMS Method A: m/z 414 (M+H)⁺ (ES+), at 2.29 min, UV active | |
| 1-10 | 5-amino-8-(2,6-dimethyl-4-pyridyl)-2-(oxazol-4-ylmethyl)-7-phenyl-[1,2,4]triazolo[4,3-c]pyrimidin-3-one | Synthetic route e; | 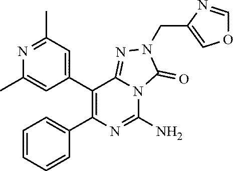 [SMILES: Cc1cc(-c2c(-c3ccccc3)nc(N)n3c(=O)n(Cc4cocn4)nc23)cc(C)n1] |
| | | Intermediates 6 & 10 | |
| | | ¹H NMR (400 MHz, DMSO-d6) δ: 8.35 (s, 1H), 8.09 (s, 1H), 7.26 (s, 5H), 6.81 (s, 2H), 4.93 (s, 2H), 2.29 (s, 6H). | |
| | | Exchangeable —NH₂ protons were not observed | |
| | | LCMS Method A: m/z 414 (M+H)⁺ (ES+), at 2.22 min, UV active | |
| 1-11 | 5-amino-8-(2,6-dimethyl-4-pyridyl)-2-[(1-methylimidazol-2-yl)methyl]-7-phenyl-[1,2,4]triazolo[4,3-c]pyrimidin-3-one | Synthetic route e | 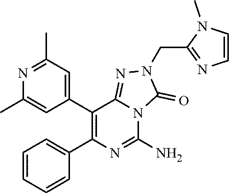 [SMILES: Cc1cc(-c2c(-c3ccccc3)nc(N)n3c(=O)n(Cc4nccn4C)nc23)cc(C)n1] |
| | | Intermediates 6 & 11 | |
| | | ¹H NMR (400 MHz, DMSO-d6) δ: 7.27-7.26 (m, 5H), 7.15 (s, 1H), 6.80 (d, J = 9.2 Hz, 3H), 5.09 (s, 2H), 3.70 (s, 3H), 2.34 (s, 6H). | |
| | | Exchangeable —NH₂ Protons were not observed. | |
| | | LCMS Method A: m/z 427 (M+H)⁺ (ES+), at 1.90 min, UV active | |
| 1-12 | 5-amino-8-(2,6-dimethyl-4-pyridyl)-2-[(2-methylpyrazol-3-yl)methyl]-7-phenyl-[1,2,4]triazolo[4,3-c]pyrimidin-3-one | Synthetic route f | 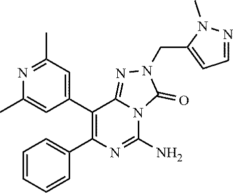 [SMILES: Cc1cc(-c2c(-c3ccccc3)nc(N)n3c(=O)n(Cc4ccnn4C)nc23)cc(C)n1] |
| | | ¹H NMR (400 MHz, DMSO-d6) δ: 7.33 - 7.26 (m, 6H), 6.80 (s, 2H), 6.22 (d, J = 2.0 Hz, 1H), 5.10 (s, 2H), 3.87 (s, 3H), 2.28 (s, 6H). | |
| | | Exchangeable —NH₂ protons were not observed. | |
| | | LCMS Method A: m/z 427 (M+H)⁺ (ES+), at 2.38 min, UV active | |
| 1-13 | 5-amino-8-(2,6-dimethyl-4-pyridyl)-2-(isoxazol-3-ylmethyl)-7-phenyl-[1,2,4]triazolo[4,3-c]pyrimidin-3-one | Synthetic route f | 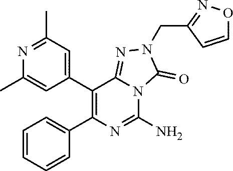 [SMILES: Cc1cc(-c2c(-c3ccccc3)nc(N)n3c(=O)n(Cc4ccon4)nc23)cc(C)n1] |
| | | Intermediates 6 & 13 | |
| | | ¹H NMR (400 MHz, DMSO-d6) δ: 8.91 (d, J = 1.8 Hz, 1H), 7.29-7.26 (m, 5H), 6.82 (s, 2H), 6.57 (d, J = 1.8 Hz, 1H), 5.15 (s, 2H), 2.29 (s, 6H). | |
| | | Exchangeable —NH₂ protons were not observed. | |
| | | LCMS Method A: m/z 414 (M+H)⁺ (ES+), at 2.38 min, UV active | |
| 1-14 | 5-amino-8-(2,6-dimethyl-4-pyridyl)-2-[(5-methylisoxazol-3-yl)methyl]-7-phenyl-[1,2,4]triazolo[4,3-c]pyrimidin-3-one | Synthetic route f (purified by prep HPLC method D) | 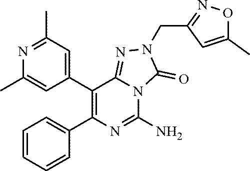 |
| | | Intermediates 6 & 14 | |
| | | ¹H NMR (400 MHz, DMSO-d6) δ:7.28 - 7.27 (m, 5H), 6.81 (s, 2H), 6.20 (s, 1H), 5.06 (s, 2H), 2.38 (s, 3H), 2.29 (s, 6H). | |
| | | Exchangeable —NH₂ protons were not observed. | |
| | | LCMS Method A: m/z 428 (M+H)⁺ (ES+), at 2.61 min, UV active | |
| 1-15 | 5-amino-8-(2,6-dimethyl-4-pyridyl)-2-[(2-methyloxazol-4-yl)methyl]-7-phenyl-[1,2,4]triazolo[4,3-c]pyrimidin-3-one | Synthetic route f | 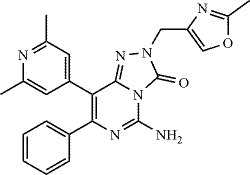 |
| | | Intermediates 6 & 15 | |
| | | ¹H NMR (400 MHz, DMSO-d6) δ: 7.90 (s, 1H), 7.32 - 7.18 (m, 5H), 6.80 (s, 2H), 4.84 (s, 2H), 2.35 (s, 3H), 2.27 (s, 6H). | |
| | | Exchangeable —NH₂ protons were not observed. | |
| | | LCMS Method A: m/z 428 (M+H)⁺ (ES+), at 2.28 min, UV active | |
| 1-16 | 5-amino-8-(2,6-dimethyl-4-pyridyl)-2-[(1-methylpyrazol-3-yl)methyl]-7-phenyl-[1,2,4]triazolo[4,3-c]pyrimidin-3-one | Synthetic route g | 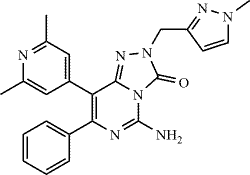 |
| | | ¹H NMR (400 MHz, DMSO-d6) δ: 8.22 (s, 2H), 7.61 (d, J = 2.4 Hz, 1H), 7.27-7.25 (m, 5H), 6.81 (s, 2H), 6.13 (d, J = 2.4 Hz, 1H), 4.91 (s, 2H), 3.77 (s, 3H), 2.29 (s, 6H). | |
| | | LCMS Method A: m/z 427 (M+H)⁺ (ES+), at 2.26 min, UV active | |
| 1-17 | 5-amino-8-(2,6-dimethyl-4-pyridyl)-2-[(5-methyl-1,3,4-oxadiazol-2-yl)methyl]-7-phenyl-[1,2,4]triazolo[4,3-c]pyrimidin-3-one | Synthetic route e (purified by normal phase Biotage 0-100% EA/pet ether) | 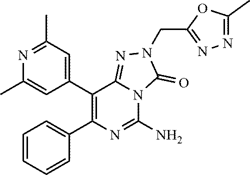 |
| | | Intermediates 6 & 17 | |
| | | ¹H NMR (400 MHz, DMSO-d6) δ: 7.70 (s, 2H), 7.31 - 7.22 (m, 5H), 6.80 (s, 2H), 5.31 (s, 2H), 2.48 (s, 3H), 2.28 (s, 6H). | |
| | | LCMS Method A: m/z 429 (M+H)⁺ (ES+), at 2.05 min, UV active | |
| 1-18 | 5-amino-8-(2,6-dimethyl-4-pyridyl)-2-[(3-methylimidazol-4-yl)methyl]-7-phenyl-[1,2,4]triazolo[4,3-c]pyrimidin-3-one | Synthetic route d (Purified by prep-HPLC method A) | 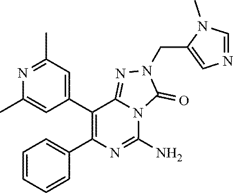 [SMILES: Cc1cc(-c2c(-c3ccccc3)nc(N)n3c(=O)n(Cc4cncn4C)nc23)cc(C)n1] |
| | | Intermediates 6 & 18 | |
| | | ¹H NMR (400 MHz, DMSO-d6) δ: 7.59 (s, 1H), 7.29 - 7.25 (m, 5H), 6.90 (s, 1H), 6.79 (s, 2H), 5.00 (s, 2H), 3.68 (s, 3H), 2.27 (s, 6H). | |
| | | Exchangeable —NH₂ protons were not observed. | |
| | | LCMS Method A: m/z 427 (M+H)⁺ (ES+), at 1.79 min, UV active | |
| 1-19 | 5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one | Synthetic route h | 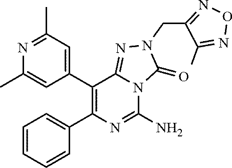 [SMILES: Cc1cc(-c2c(-c3ccccc3)nc(N)n3c(=O)n(Cc4nonc4C)nc23)cc(C)n1] |
| | | ¹H NMR (400 MHz, DMSO-d6) δ: 8.78 (s, 1H), 7.80 (s, 1H), 7.37-7.31 (m, 5H), 7.29 (s, 2H), 5.31 (s, 2H), 2.51 (s, 9H). | |
| | | LCMS Method A: m/z 427 (M-H)⁻ (ES-), at 2.18 min, UV active | |
| 1-20 | 5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one | Synthetic route i | 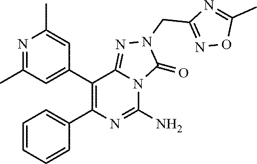 |
| | | ¹H NMR (400 MHz, DMSO-d6) δ: 7.88 (s, 2H), 7.29 - 7.25 (m, 5H), 6.80 (s, 2H), 5.18 (s, 2H), 2.57 (s, 3H), 2.28 (s, 6H). | |
| | | LCMS Method A: m/z 429 (M+H)⁺ (ES+), at 2.24 min, UV active | |
| 1-21 | 5-amino-8-(2,6-dimethyl-4-pyridyl)-2-[(3-methyl-1,2,4-oxadiazol-5-yl)methyl]-7-phenyl-[1,2,4]triazolo[4,3-c]pyrimidin-3-one | Synthetic route e (purified by column chromatography, 40% EA/pet ether) | 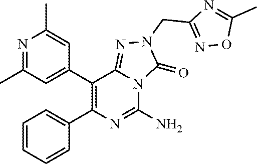 |
| | | Intermediates 6 & 20 | |
| | | ¹H NMR (400 MHz, DMSO-d6) δ: 7.87 (s, 2H), 7.31 - 7.25 (m, 5H), 6.81 (s, 2H), 5.44 (s, 2H), 2.33 (s, 3H), 2.28 (s, 6H). | |
| | | LCMS Method A: m/z 429 (M+H)⁺ (ES+), at 2.32 min, UV active | |
| 1-22 | 5-amino-8-(2,6-dimethyl-4-pyridyl)-2-[(5-methyloxazol-4-yl)methyl]-7-phenyl-[1,2,4]triazolo[4,3-c]pyrimidin-3-one | Synthetic route f (step 1 reaction using NaH as base in THF) | 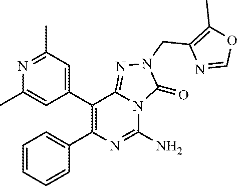 [SMILES: Cc1cc(-c2c(-c3ccccc3)nc(N)n3c(=O)n(Cc4ncoc4C)nc23)cc(C)n1] |
| | | Intermediates 6 & 21 | |
| | | ¹H NMR (400 MHz, DMSO-d6) δ: 8.16 (s, 1H), 77.35 - 7.18 (m, 5H), 6.80 (s, 2H), 4.87 (s, 2H), 2.35 (s, 3H), 2.28 (s, 6H). | |
| | | Exchangeable —NH₂ protons were not observed. | |
| | | LCMS Method A: m/z 428 (M+H)⁺ (ES+), at 2.37 min, UV active | |
| 1-23 | 5-amino-8-(2,6-dimethyl-4-pyridyl)-2-(1H-imidazol-5-ylmethyl)-7-phenyl-[1,2,4]triazolo[4,3-c]pyrimidin-3-one | Synthetic route g | 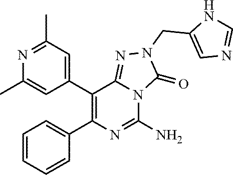 [SMILES: Cc1cc(-c2c(-c3ccccc3)nc(N)n3c(=O)n(Cc4cnc[nH]4)nc23)cc(C)n1] |
| | | Intermediates 6 & 22 | |
| | | ¹H NMR (400 MHz, DMSO-d6) δ: 12.09 (s, 1H), 8.34 (s, 2H), 7.59 (s, 1H), 7.35 - 7.17 (m, 5H), 7.01 (s, 1H), 6.79 (s, 2H), 4.88 (s, 2H), 2.27 (s, 6H). | |
| | | LCMS Method E: m/z 413 (M+H)⁺ (ES+), at 4.14 min, UV active | |
| 1-24 | 5-amino-8-(2,6-dimethyl-4-pyridyl)-2-(1H-imidazol-2-ylmethyl)-7-phenyl-[1,2,4]triazolo[4,3-c]pyrimidin-3-one | Synthetic route g (purified by Prep HPLC method B) | 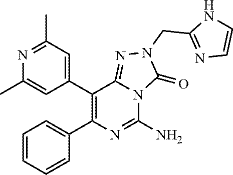 [SMILES: Cc1cc(-c2c(-c3ccccc3)nc(N)n3c(=O)n(Cc4ncc[nH]4)nc23)cc(C)n1] |
| | | Intermediates 6 & 23 | |
| | | ¹H NMR (400 MHz, DMSO-d6) δ: 11.92 (s, 1H), 8.43 (s, 2H), 7.30 - 7.25 (m, 5H), 6.98 - 6.78 (m, 4H), 5.01 (s, 2H), 2.26 (s, 6H). | |
| | | LCMS Method E: m/z 412 (M+H)⁺ (ES+), at 4.24 min, UV active | |
| 1-25 | 5-amino-8-(2,6-dimethyl-4-pyridyl)-7-phenyl-2-(1H-pyrazol-5-ylmethyl)-[1,2,4]triazolo[4,3-c]pyrimidin-3-one | Synthetic route g | 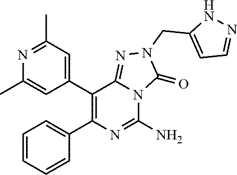 [SMILES: Cc1cc(-c2c(-c3ccccc3)nc(N)n3c(=O)n(Cc4ccn[nH]4)nc23)cc(C)n1] |
| | | Intermediates 6 & 24 | |
| | | ¹H NMR (400 MHz, DMSO-d6) δ: 12.71 (s, 1H), 7.66 (s, 1H), 7.28-7.22 (m, 5H), 6.79 (s, 2H), 6.17 (s, 1H), 4.96 (s, 2H), 2.27 (s, 6H). | |
| | | Exchangeable —NH₂ protons were not observed. | |
| | | LCMS Method E: m/z 413 (M+H)⁺ (ES+), at 4.42 min, UV active | |
| 1-26 | 5-amino-8-(2,6-dimethyl-4-pyridyl)-7-phenyl-2-(2H-tetrazol-5-ylmethyl)-[1,2,4]triazolo[4,3-c]pyrimidin-3-one | Synthetic route j | 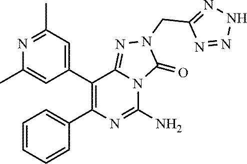 [SMILES: Cc1cc(-c2c(-c3ccccc3)nc(N)n3c(=O)n(Cc4nn[nH]n4)nc23)cc(C)n1] |
| | | ¹H NMR (400 MHz, DMSO-d6) δ: 8.31 (s, 2H), 7.27 - 7.25 (m, 5H), 7.02 (s, 1H), 6.78 (s, 2H), 5.26 (s, 2H), 2.27 (s, 6H). | |
| | | LCMS Method B: m/z 415 (M+H)⁺ (ES+), at 1.57 min, UV active | |
| 1-27 | 5-amino-8-(2,6-dimethyl-4-pyridyl)-2-(1,3,4-oxadiazol-2-ylmethyl)-7-phenyl-[1,2,4]triazolo[4,3-c]pyrimidin-3-one | Synthetic route k | 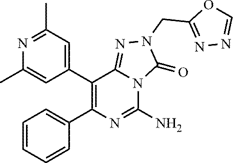 [SMILES: Cc1cc(-c2c(-c3ccccc3)nc(N)n3c(=O)n(Cc4nnco4)nc23)cc(C)n1] |
| | | ¹H NMR (400 MHz, DMSO-d6) δ: 9.27 (s, 1H), 7.28 - 7.24 (m, 5H), 6.79 (s, 2H), 5.41 (s, 2H), 2.28 (s, 6H). | |
| | | Exchangeable —NH₂ protons were not observed | |
| | | LCMS Method A: m/z 415 (M+H)⁺ (ES+), at 1.89 min, UV active | |
| 1-28 | 5-amino-8-(2,6-dimethyl-4-pyridyl)-2-[(4-methyl-1,2,4-triazol-3-yl)methyl]-7-phenyl-[1,2,4]triazolo[4,3-c]pyrimidin-3-one | Synthetic route g | 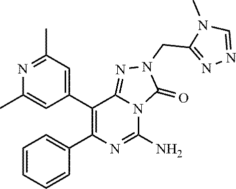 [SMILES: Cc1cc(-c2c(-c3ccccc3)nc(N)n3c(=O)n(Cc4nncn4C)nc23)cc(C)n1] |
| | | Intermediates 6 & 25 | |
| | | ¹H NMR (400 MHz, DMSO-d6) δ: 8.46 (s, 1H), 7.26-7.25 (m, 5H), 6.78 (s, 2H), 5.21 (s, 2H), 3.69 (s, 3H), 2.27 (s, 6H). | |
| | | Exchangeable —NH₂ and protons were not observed. | |
| | | LCMS Method A: m/z 428 (M+H)⁺ (ES+), at 1.48 min, UV active | |
| 1-29 | 5-amino-8-(2,6-dimethyl-4-pyridyl)-2-[(5-methyl-1H-triazol-4-yl)methyl]-7-phenyl-[1,2,4]triazolo[4,3-c]pyrimidin-3-one | Synthetic route l | 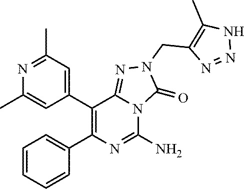 [SMILES: Cc1cc(-c2c(-c3ccccc3)nc(N)n3c(=O)n(Cc4nn[nH]c4C)nc23)cc(C)n1] |
| | | ¹H NMR (400 MHz, DMSO-d6) δ: 14.91-14.50 (m, 1H), 7.28 - 7.24 (m, 5H), 6.78 (s, 2H), 5.03 (s, 2H), 2.29 - 2.25 (m, 9H). | |
| | | Exchangeable —NH₂ protons were not observed. | |
| | | LCMS Method A: m/z 428 (M+H)⁺ (ES+), at 1.58 min, UV active | |
| 1-30 | 5-amino-8-(2,6-dimethyl-4-pyridyl)-2-[(2-methyltriazol-4-yl)methyl]-7-phenyl-[1,2,4]triazolo[4,3-c]pyrimidin-3-one | Synthetic route m | 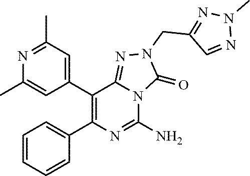 |
| | | ¹H NMR (400 MHz, DMSO-d6) δ: 8.50-7.90 (m, 2H), 7.68 (s, 1H), 7.29-7.24 (m, 5H), 6.79 (s, 2H), 5.06 (s, 2H), 4.10 (s, 3H), 2.28 (s, 6H). | |
| | | Structure was confirmed by NOE studies. | |
| | | LCMS Method D: m/z 428 (M+H)⁺ (ES+), at 1.07 min, UV active | |
| 1-31 | 5-amino-8-(2,6-dimethyl-4-pyridyl)-7-(4-fluorophenyl)-2-[(5-methyloxazol-4-yl)methyl]-[1,2,4]triazolo[4,3-c]pyrimidin-3-one | Synthetic route a-Step 2 | 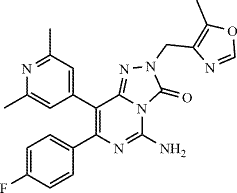 [SMILES: Cc1cc(-c2c(-c3ccc(F)cc3)nc(N)n3c(=O)n(Cc4ncoc4C)nc23)cc(C)n1] |
| | | Intermediates 27 & 3 | |
| | | ¹H NMR (400 MHz, DMSO-d6) δ: 8.16 (s, 1H), 7.32-7.28 (m, 2H), 7.13-7.08 (m, 2H), 6.81 (s, 2H), 4.86 (s, 2H), 2.35 (s, 3H), 2.30 (s, 6H). | |
| | | Exchangeable —NH₂ protons were not observed. | |
| | | LCMS Method C: m/z 446 (M+H)⁺ (ES+), at 1.74 min, UV active | |
| 1-32 | 5-amino-2-[(2,5-dimethyloxazol-4-yl)methyl]-8-(2,6-dimethyl-4-pyridyl)-7-(4-fluorophenyl)-[1,2,4]triazolo[4,3-c]pyrimidin-3-one | Synthetic route a-Step 2 | 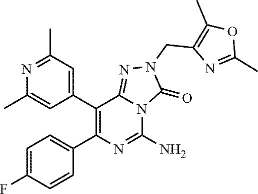 |
| | | Intermediates 28 & 3 | |
| | | ¹H NMR (400 MHz, DMSO-d6) δ 7.34 - 7.24 (m, 2H), 7.15 - 7.04 (m, 2H), 6.81 (s, 2H), 4.78 (s, 2H), 2.32 - 2.27 (m, 12H). | |
| | | Exchangeable —NH₂ protons were not observed. | |
| | | LCMS Method D: m/z 458 (M-H)⁻ (ES-), at 5.03 min, UV active | |
| 1-33 | 5-amino-2-[[1-benzyl-3-(3-methoxyphenyl)pyrazol-4-yl]methyl]-8-(2,6-dimethyl-4-pyridyl)-7-phenyl-[1,2,4]triazolo[4,3-c]pyrimidin-3-one | Synthetic route d | 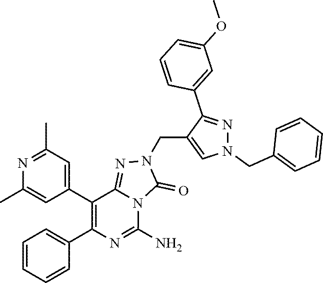 [SMILES: COc1cccc(-c2nn(Cc3ccccc3)cc2Cn2nc3c(-c4cc(C)nc(C)c4)c(-c4ccccc4)nc(N)n3c2=O)c1] |
| | | Intermediates 6 & 30 | |
| | | ¹H NMR (400 MHz, Chloroform-d) δ 7.52 (s, 1H), 7.43 - 7.24 (m, 13H), 6.96 - 6.70 (m, 3H), 5.30 (s, 2H), 5.02 (s, 2H), 3.74 (s, 3H), 2.39 (s, 6H). | |
| | | Exchangeable —NH₂ protons were not observed. | |
| | | LCMS Method F: m/z 609 (M+H)⁺ (ES+), at 1.25 min, UV active | |
| 1-34 | 5-amino-8-(2,6-dimethyl-1-oxido-pyridin-1-ium-4-yl)-2-[(5-methyloxazol-4-yl)methyl]-7-phenyl-[1,2,4]triazolo[4,3-c]pyrimidin-3-one | Synthetic route n | 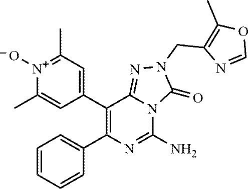 |
| | | ¹H NMR (400 MHz, DMSO-d6) δ 8.16 (s, 1H), 7.41 - 7.21 (m, 5H), 7.09 (s, 2H), 4.88 (s, 2H), 2.36 (s, 3H), 2.23 (s, 6H). | |
| | | Exchangeable —NH₂ protons were not observed. | |
| | | LCMS Method C: m/z 444 (M+H)⁺ (ES+), at 1.40 min, UV active | |
| 1-35 | 5-amino-2-[(2,5-dimethyloxazol-4-yl)methyl]-8-(2,6-dimethyl-1-oxido-pyridin-1-ium-4-yl)-7-(4-fluorophenyl)-[1,2,4]triazolo[4,3-c]pyrimidin-3-one | Synthetic route n | 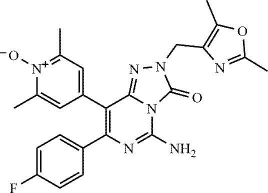 |
| | | Example 1-32 | |
| | | ¹H NMR (400 MHz, DMSO-d6) δ: 7.38-7.35 (m, 2H), 7.16-7.11 (m, 4H), 4.83 - 4.77 (m, 2H), 2.32 (s, 12H). | |
| | | Exchangeable —NH₂ protons were not observed. | |
| | | LCMS Method C: m/z 476 (M+H)⁺ (ES+), at 1.49 min, UV active | |
| 1-36 | 5-amino-8-(2,6-dimethyl-1-oxido-pyridin-1-ium-4-yl)-2-[(1-methylimidazol-2-yl)methyl]-7-phenyl-[1,2,4]triazolo[4,3-c]pyrimidin-3-one | Synthetic route n | 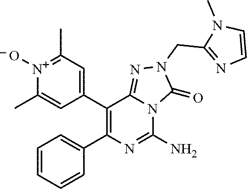 |
| | | Example 1-11 | |
| | | ¹H NMR (400 MHz, DMSO-d6) δ: 7.33 - 7.29 (m, 5H), 7.12 (s, 1H), 7.08 (s, 2H), 6.78 (s, 1H), 5.08 (s, 2H), 3.70 (s, 3H), 2.22 (s, 6H). | |
| | | Exchangeable —NH₂ protons were not observed. | |
| | | LCMS Method C: m/z 443 (M+H)⁺ (ES+), at 0.96 min, UV active | |
| 1-37 | 5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one | Synthetic route n (Purified by flash column chromatography, eluted with 0-3% MeOH in DCM) | 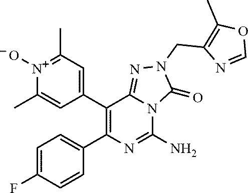 |
| | | Example 1-31 | |
| | | ¹H NMR (400 MHz, DMSO-d6) δ 8.16 (s, 1H), 7.41 - 7.33 (m, 2H), 7.19 -7.08 (m, 5H), 4.87 (s, 2H), 2.35 (s, 3H), 2.25 (s, 6H). | |
| | | One exchangeable —NH proton was not observed. | |
| | | LCMS Method C: m/z 462 (M+H)⁺ (ES+), at 1.92 min, UV active | |
| 1-38 | 5-amino-8-(2-m ethoxy-6-methyl-4-pyridyl)-2-[(1-methylimidazol-2-yl)methyl]-7-phenyl-[1,2,4]triazolo[4,3-c]pyrimidin-3-one | Synthetic route a-Step 2 | 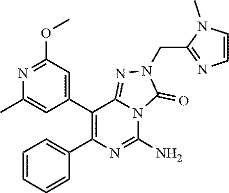 [SMILES: COc1cc(-c2c(-c3ccccc3)nc(N)n3c(=O)n(Cc4nccn4C)nc23)cc(C)n1] |
| | | Intermediates 31 and 32 | |
| | | ¹H NMR (400 MHz, DMSO-d6) δ: 7.29-7.27 (m, 5H), 7.12 (s, 1H), 6.77 (d, J = 0.8 Hz, 1H), 6.55 (s, 1H), 6.38 (s, 1H), 5.07 (s, 2H), 3.69 (s, 3H), 3.68 (s, 3H), 2.51 (s, 3H). | |
| | | Exchangeable —NH2 protons were not observed. | |
| | | LCMS Method C: m/z 443 (M+H)⁺ (ES+), at 1.42 min, UV active | |
| 1-39 | 5-amino-8-[2-(hydroxymethyl)-6-methyl-4-pyridyl]-2-[(5-methyloxazol-4-yl)methyl]-7-phenyl-[1,2,4]triazolo[4,3-c]pyrimidin-3-one | Synthetic route o | 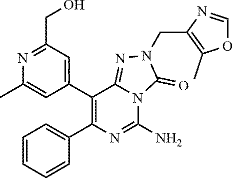 [SMILES: Cc1cc(-c2c(-c3ccccc3)nc(N)n3c(=O)n(Cc4ncoc4C)nc23)cc(CO)n1] |
| | | ¹H NMR (400 MHz, DMSO-d6) δ: 8.25 (s, 1H), 7.28-7.23 (m, 7H), 4.87 (s, 2H), 4.55 (s, 2H), 2.34 (s, 6H). | |
| | | Exchangeable —NH₂ and —OH protons were not observed. | |
| | | LCMS Method C: m/z 444 (M+H)⁺ (ES+), at 0.89 min, UV active | |
| 1-40 | 5-amino-8-[2-(hydroxymethyl)-6-methyl-4-pyridyl]-2-(oxazol-2-ylmethyl)-7-phenyl-[1,2,4]triazolo[4,3-c]pyrimidin-3-one | Synthetic route p | 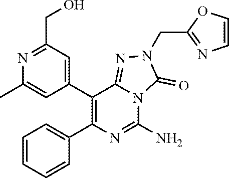 [SMILES: Cc1cc(-c2c(-c3ccccc3)nc(N)n3c(=O)n(Cc4ncco4)nc23)cc(CO)n1] |
| | | ¹H NMR (400 MHz, DMSO-d6) δ: 8.11 (s, 1H), 7.29-7.23 (m, 5H), 7.20 (s, 1H), 7.09 (s, 1H),6.83 (s, 1H), 5.27-5.24 (m, 1H), 5.20 (s, 2H), 4.41 (d, J 5.6 Hz, 2H), 2.29 (s, 3H). | |
| | | Exchangeable —NH₂ Protons were not observed. | |
| | | LCMS Method A: m/z 430 (M+H)⁺ (ES+), at 1.55 min, UV active | |
| 1-41 | 5-amino-8-[2-(hydroxymethyl)-6-methyl-4-pyridyl]-2-[(1-methylimidazol-2-yl)methyl]-7-phenyl-[1,2,4]triazolo[4,3-c]pyrimidin-3-one | Synthetic route q | 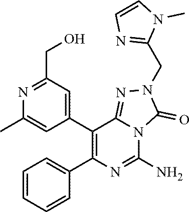 |
| | | ¹H NMR (400 MHz, DMSO-d6) δ: 7.27-7.26 (m, 5H), 7.15-7.05 (m, 2H), 6.81 (s, 1H), 6.77 (d, J = 1.2 Hz, 1H), 5.24 (d, J = 5.0 Hz, 1H), 5.06 (s, 2H), 4.41 (d, J = 5.0 Hz, 2H), 3.69 (s, 3H), 2.28 (s, 3H). | |
| | | Exchangeable —NH₂ Protons were not observed. | |
| | | LCMS Method A: m/z 443 (M+H)⁺ (ES+), at 1.63 min, UV active | |
| 1-42 | 5-amino-8-[2-(hydroxymethyl)-6-methyl-4-pyridyl]-2-(oxazol-4-ylmethyl)-7-phenyl-[1,2,4]triazolo[4,3-c]pyrimidin-3-one | Synthetic route r | 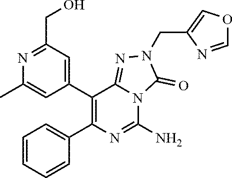 [SMILES: Cc1cc(-c2c(-c3ccccc3)nc(N)n3c(=O)n(Cc4cocn4)nc23)cc(CO)n1] |
| | | ¹H NMR (400 MHz, DMSO-d6) δ:8.35 (d, J = 0.8 Hz, 1H), 8.08 (d, J = 0.8 Hz, 1H), 7.29-7.22 (m, 5H), 7.10 (s, 1H), 6.83 (s, 1H), 5.26-5.23 (m, 1H), 4.93 (s, 2H), 4.41 (d, J = 5.2 Hz, 2H), 2.29 (s, 3H). | |
| | | Exchangeable —NH₂ protons were not observed. | |
| | | LCMS Method A: m/z 430 (M+H)⁺ (ES+), at 1.51 min, UV active | |
| 1-43 | 5-amino-8-[2-(hydroxymethyl)-6-methyl-4-pyridyl]-2-[(5-methylisoxazol-3-yl)methyl]-7-phenyl-[1,2,4]triazolo[4,3-c]pyrimidin-3-one | Synthetic route s | 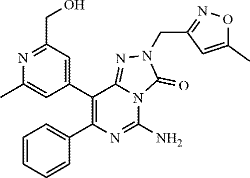 |
| | | ¹H NMR (400 MHz, DMSO-d6) δ: 7.28-7.24 (m, 5H), 7.12 (s, 1H), 6.80 (s, 1H), 6.21 (s, 1H), 5.26 (t, J = 5.6 Hz, 1H), 5.03 (s, 2H), 4.42 (d, J = 5.6 Hz, 2H), 2.36 (s, 3H), 2.28 (s, 3H). | |
| | | Exchangeable NH₂— Protons were not observed. | |
| | | LCMS Method A: m/z 444 (M+H)⁺ (ES+), at 1.92 min, UV active | |
| 1-44 | 5-amino-2-[(3,5-dimethylimidazol-4-yl)methyl]-8-[2-(hydroxymethyl)-6-methyl-4-pyridyl]-7-phenyl-[1,2,4]triazolo[4,3-c]pyrimidin-3-one | Synthetic route s | 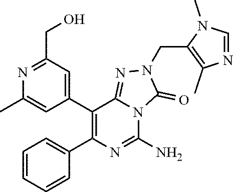 [SMILES: Cc1cc(-c2c(-c3ccccc3)nc(N)n3c(=O)n(Cc4c(C)ncn4C)nc23)cc(CO)n1] |
| | | Intermediates 33 & 37 (step 1) | |
| | | ¹H NMR (400 MHz, DMSO-d6) δ: 7.46 (s, 1H), 7.30 - 7.27 (m, 5H), 7.10 (s, 1H), 6.83 (s, 1H), 5.20 (t, J = 5.6 Hz, 1H), 4.95 (s, 2H), 4.41 (d, J = 5.6 Hz, 2H), 3.66 (s, 3H), 2.29 (s, 3H), 2.16 (s, 3H). | |
| | | Exchangeable NH₂— Protons were not observed. | |
| | | LCMS Method C: m/z 457 (M+H)⁺ (ES+), at 0.71 min, UV active | |
| 1-45 | 5-amino-8-[2-(hydroxymethyl)-6-methyl-4-pyridyl]-2-[(1-methylpyrazol-3-yl)methyl]-7-phenyl-[1,2,4]triazolo[4,3-c]pyrimidin-3-one | Synthetic route s | 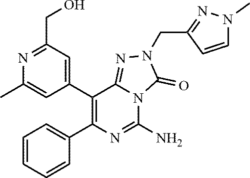 |
| | | Intermediates 33 & 16 (step 1) | |
| | | ¹H NMR (400 MHz, DMSO-d6) δ: 8.25 (s, 2H), 7.64 - 7.56 (m, 1H), 7.31-7.26 (m, 5H), 7.11 (s, 1H), 6.83 (s, 1H), 6.17 - 6.11 (m, 1H), 5.28 (s, 1H), 4.92 (s, 2H), 4.43 (s, 2H), 3.78 (s, 3H), 2.29 (s, 3H). | |
| | | LCMS Method D: m/z 443 (M+H)⁺ (ES+), at 2.09 min, UV active | |
| 1-46 | 5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one | Synthetic route t | 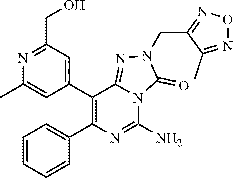 [SMILES: Cc1cc(-c2c(-c3ccccc3)nc(N)n3c(=O)n(Cc4nonc4C)nc23)cc(CO)n1] |
| | | ¹H NMR (400 MHz, DMSO-d6) δ: 7.26 (s, 5H), 7.08 (s, 1H), 6.82 (s, 1H), 5.29 (s, 2H), 5.24 (t, J = 5.6 Hz, 1H), 4.41 (d, J = 5.6 Hz, 2H), 2.40 (s, 3H), 2.35 (s, 3H). | |
| | | Exchangeable NH₂— Protons were not observed. | |
| | | LCMS Method B: m/z 445 (M+H)⁺ (ES+), at 1.19 min, UV active | |
| 1-47 | 5-amino-8-[2-(hydroxymethyl)-6-methyl-4-pyridyl]-2-[(5-methyloxazol-4-yl)methyl]-7-(2,3,4,5,6-pentadeuteriophenyl)-[1,2,4]triazolo[4,3-c]pyrimidin-3-one | Synthetic route s | 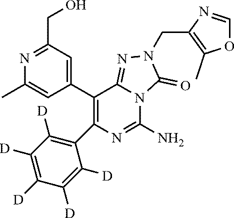 [SMILES: [2H]c1c([2H])c([2H])c(-c2nc(N)n3c(=O)n(Cc4ncoc4C)nc3c2-c2cc(C)nc(CO)c2)c([2H])c1[2H]] |
| | | Intermediates 38 & 41 (step 1) | |
| | | ¹H NMR (400 MHz, DMSO-d6) δ: 8.15 (s, 1H), 7.09 (s, 1H), 6.83 (s, 1H), 5.23 (t, J = 5.6 Hz, 1H), 4.86 (s, 2H), 4.41 (d, J = 5.6 Hz, 2H), 2.35 (s, 3H), 2.29 (s, 3H). | |
| | | Exchangeable NH₂— Protons were not observed. | |
| | | LCMS Method C: m/z 449 (M+H)⁺ (ES+), at 1.08 min, UV active | |
| 1-48 | 5-amino-8-[2-(hydroxymethyl)-6-methyl-4-pyridyl]-2-[(1-methylimidazol-2-yl)methyl]-7-(2,3,4,5,6-pentadeuteriophenyl)-[1,2,4]triazolo[4,3-c]pyrimidin-3-one | Synthetic route o (step 1 work-up diluted with ice water and solid filtered) | 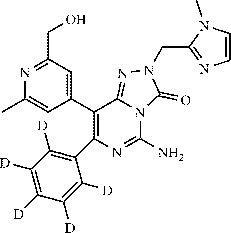 [SMILES: [2H]c1c([2H])c([2H])c(-c2nc(N)n3c(=O)n(Cc4nccn4C)nc3c2-c2cc(C)nc(CO)c2)c([2H])c1[2H]] |
| | | Intermediates 38 & 11 (step 1) | |
| | | ¹H NMR (400 MHz, DMSO-d6) δ: 7.10 (d, J = 8.0 Hz, 2H), 6.81 (s, 1H), 6.77 (s, 1H), 5.24-5.22 (m, 1H), 5.06 (s, 2H), 4.41 (d, J = 4.0 Hz, 2H), 3.69 (s, 3H), 2.28 (s, 3H). | |
| | | Exchangeable NH₂— Protons were not observed. | |
| | | LCMS Method D: m/z 448 (M+H)⁺ (ES+), at 1.71 min, UV active | |
| 1-49 | 5-amino-8-[2-(hydroxymethyl)-6-methyl-4-pyridyl]-2-(oxazol-4-ylmethyl)-7-(2,3,4,5,6-pentadeuteriophenyl)-[1,2,4]triazolo[4,3-c]pyrimidin-3-one | Synthetic route s | 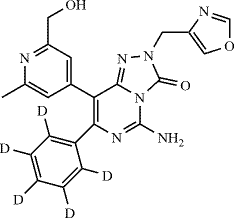 [SMILES: [2H]c1c([2H])c([2H])c(-c2nc(N)n3c(=O)n(Cc4cocn4)nc3c2-c2cc(C)nc(CO)c2)c([2H])c1[2H]] |
| | | Intermediates 38 & 10 (step 1) | |
| | | ¹H NMR (400 MHz, DMSO-d6) δ: 8.18 (s, 1H), 7.95 (s, 1H), 7.27 (s, 1H), 6.94 (s, 1H), 5.02 (s, 2H), 4.56 (s, 2H), 2.36 (s, 3H). | |
| | | Exchangeable —NH2 and —OH Protons were not observed. | |
| | | LCMS Method C: m/z 435 (M+H)⁺ (ES+), at 0.98 min, UV active | |
| 1-50 | 5-amino-7-(4-fluorophenyl)-8-[2-(hydroxymethyl)-6-methyl-4-pyridyl]-2-[(1-methylimidazol-2-yl)methyl]-[1,2,4]triazolo[4,3-c]pyrimidin-3-one | Synthetic route s | 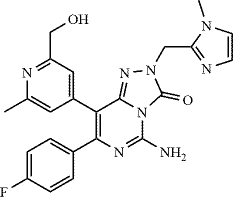 [SMILES: Cc1cc(-c2c(-c3ccc(F)cc3)nc(N)n3c(=O)n(Cc4nccn4C)nc23)cc(CO)n1] |
| | | Intermediates 42 & 11 (step 1) | |
| | | ¹H NMR (400 MHz, DMSO-d6) δ: 7.32-7.28 (m, 2H), 7.12-7.07 (m, 4H), 6.85 (s, 1H), 6.77 (s, 1H), 5.24 (t, J = 5.8 Hz, 1H), 5.06 (s, 2H), 4.42 (d, J = 5.8 Hz, 2H), 3.69 (s, 3H), 2.33 (s, 3H). | |
| | | Exchangeable —NH₂ protons were not observed. | |
| | | LCMS Method C: m/z 461 (M+H)⁺ (ES+), at 1.81 min, UV active | |
| 1-51 | 5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one | Synthetic route s | 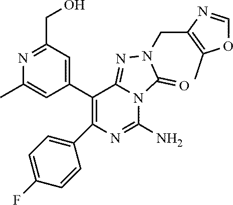 [SMILES: Cc1cc(-c2c(-c3ccc(F)cc3)nc(N)n3c(=O)n(Cc4ncoc4C)nc23)cc(CO)n1] |
| | | Intermediates 42 & 21 (step 1) | |
| | | ¹H NMR (400 MHz, DMSO-d6) δ: 8.15 (s, 1H), 7.32-7.28 (m, 2H), 7.13 - 7.04 (m, 3H), 6.87 (s, 1H), 5.24 (t, J = 5.6 Hz, 1H), 4.86 (s, 2H), 4.42 (d, J = 6.0 Hz, 2H), 2.35 (s, 3H), 2.33 (s, 3H). | |
| | | Exchangeable —NH₂ protons were not observed. | |
| | | LCMS Method C: m/z 462 (M+H)⁺ (ES+), at 1.13 min, UV active | |
| 1-52 | 5-amino-7-(4-fluorophenyl)-8-[2-(hydroxymethyl)-6-methyl-4-pyridyl]-2-[(1-methylpyrazol-3-yl)methyl]-[1,2,4]triazolo[4,3-c]pyrimidin-3-one | Synthetic route r | 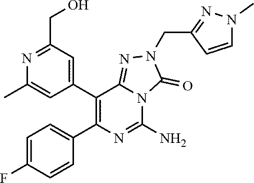 |
| | | Intermediates 26 & 43 (step 1), 34 (step 2) | |
| | | ¹H NMR (400 MHz, DMSO-d6) δ: 7.60 (d, J = 4.0 Hz, 1H), 7.31-7.27 (m, 2H), 7.11-7.06 (m, 3H), 6.88 (s, 1H), 6.18 - 6.11 (m, 1H), 5.27-5.25 (m, 1H), 4.90 (s, 2H), 4.43 (d, J = 4.0 Hz, 2H), 3.76 (s, 3H), 2.31 (s, 3H). | |
| | | Exchangeable — NH₂ protons were not observed. | |
| | | LCMS Method C: m/z 461 (M+H)⁺ (ES+), at 1.22 min, UV active | |
| 1-53 | 5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one | Synthetic route u | 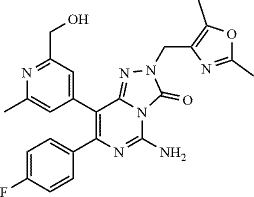 |
| | | ¹H NMR (400 MHz, DMSO-d6) δ: 7.32-7.28 (m, 2H), 7.12-7.07 (m, 3H), 6.87 (s, 1H), 5.28 (s, 1H), 4.78 (s, 2H), 4.42 (s, 2H), 2.32 (s, 3H), 2.29 (s, 6H). | |
| | | Exchangeable —NH₂ protons were not observed. | |
| | | LCMS Method C: m/z 476 (M+H)⁺ (ES+), at 1.39 min, UV active | |
| 1-54 | 5-amino-8-[2-chloro-6-(hydroxymethyl)-4-pyridyl]-2-[(5-methyloxazol-4-yl)methyl]-7-phenyl-[1,2,4]triazolo[4,3-c]pyrimidin-3-one | Synthetic route q | 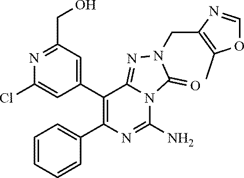 [SMILES: Cc1ocnc1Cn1nc2c(-c3cc(Cl)nc(CO)c3)c(-c3ccccc3)nc(N)n2c1=O] |
| | | Intermediates 1 & 21 (step 1), 44 (step 2) | |
| | | ¹H NMR (400 MHz, DMSO-d6) δ: 8.16 (s, 1H), 7.77 (s, 1H), 7.33-7.28 (m, 5H), 7.05 (s, 1H), 5.42 (t, J = 5.65 Hz, 1H), 4.88 (s, 2H), 4.41 (d, J = 5.65 Hz, 2H), 2.37 (s, 3H). | |
| | | Exchangeable —NH₂ Protons were not observed. | |
| | | LCMS Method A: m/z 464 (M+H)⁺ (ES+), at 2.29 min, UV active | |
| 1-55 | 5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one | Synthetic route v | 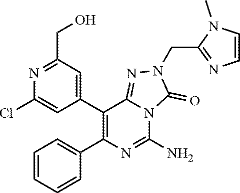 [SMILES: Cn1ccnc1Cn1nc2c(-c3cc(Cl)nc(CO)c3)c(-c3ccccc3)nc(N)n2c1=O] |
| | | ¹H NMR (400 MHz, DMSO-d6) δ: 7.34-7.29 (m, 6H), 7.12 (s, 1H), 7.03 (s, 1H), 6.78 (s, 1H), 5.44 (t, J = 5.8 Hz, 1H), 5.09 (s, 2H), 4.42 (d, J = 5.8 Hz, 2H), 3.72 (s, 3H). | |
| | | Exchangeable —NH₂ protons were not observed. | |
| | | LCMS Method C: m/z 463 (M+H)⁺ (ES+), at 1.19 min, UV active | |
| 1-56 | 5-amino-8-[2-chloro-6-(hydroxymethyl)-4-pyridyl]-2-[(1-methylimidazol-2-yl)methyl]-7-(2,3,4,5,6-pentadeuteriophenyl)-[1,2,4]triazolo[4,3-c]pyrimidin-3-one | Synthetic route v | 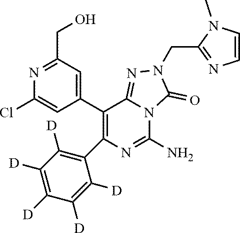 [SMILES: [2H]c1c([2H])c([2H])c(-c2nc(N)n3c(=O)n(Cc4nccn4C)nc3c2-c2cc(Cl)nc(CO)c2)c([2H])c1[2H]] |
| | | Intermediates 39 & 11 (step 1), 44 (step 2) | |
| | | ¹H NMR (400 MHz, DMSO-d6) δ: 8.60-7.50 (m, 2H), 7.27 (s, 1H), 7.11 (s, 1H), 7.02 (s, 1H), 6.77 (s, 1H), 5.42 (d, J = 6.0 Hz, 1H), 5.07 (s, 2H), 4.41 (d, J = 6.0 Hz, 2H), 3.70 (s, 3H). | |
| | | LCMS Method C: m/z 468 (M+H)⁺ (ES+), at 1.40 min, UV active | |
| 1-57 | 5-amino-8-[2-chloro-6-(hydroxymethyl)-4-pyridyl]-2-[(5-methyloxazol-4-yl)methyl]-7-(2,3,4,5,6-pentadeuteriophenyl)-[1,2,4]triazolo[4,3-c]pyrimidin-3-one | Synthetic route q | 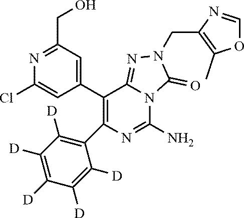 [SMILES: [2H]c1c([2H])c([2H])c(-c2nc(N)n3c(=O)n(Cc4ncoc4C)nc3c2-c2cc(Cl)nc(CO)c2)c([2H])c1[2H]] |
| | | Intermediates 39 & 21 (step 1), 44 (step 2) | |
| | | ¹H NMR (400 MHz, DMSO-d6) δ: 8.16 (s, 1H), 7.27 (s, 1H), 7.05 (s, 1H), 5.42 (t, J = 5.8 Hz, 1H), 4.88 (s, 2H), 4.41 (d, J = 5.8 Hz, 2H), 2.37 (s, 3H). | |
| | | Exchangeable —NH₂ Protons were not observed. | |
| | | LCMS Method C: m/z 469 (M+H)⁺ (ES+), at 1.72 min, UV active | |
| 1-58 | 5-amino-8-[2-chloro-6-(hydroxymethyl)-4-pyridyl]-7-(4-fluorophenyl)-2-[(1-methylimidazol-2-yl)methyl]-[1,2,4]triazolo[4,3-c]pyrimidin-3-one | Synthetic route v | 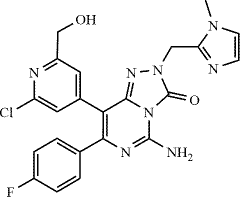 [SMILES: Cn1ccnc1Cn1nc2c(-c3cc(Cl)nc(CO)c3)c(-c3ccc(F)cc3)nc(N)n2c1=O] |
| | | Intermediates 26 & 11 (step 1), 44 (step 2) | |
| | | ¹H NMR (400 MHz, DMSO-d6) δ :7.33-7.30 (m, 2H), 7.21 (s, 1H), 7.17 ― 7.10 (m, 3H), 7.07 (s, 1H), 6.78 (d, J = 0.8 Hz, 1H), 5.47 (s, 1H), 5.06 (s, 2H), 4.40 (s, 2H), 3.71 (s, 3H). | |
| | | Exchangeable —NH₂ Protons were not observed. | |
| | | LCMS Method C: m/z 481 (M+H)⁺ (ES+), at 1.25 min, UV active | |
| 1-59 | 5-amino-8-[2-chloro-6-(hydroxymethyl)-4-pyridyl]-7-(4-fluorophenyl)-2-[(5-methyloxazol-4-yl)methyl]-[1,2,4]triazolo[4,3-c]pyrimidin-3-one | Synthetic route q | 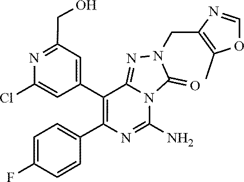 [SMILES: Cc1ocnc1Cn1nc2c(-c3cc(Cl)nc(CO)c3)c(-c3ccc(F)cc3)nc(N)n2c1=O] |
| | | Intermediates 26 & 21 (step 1), 44 (step 2) | |
| | | ¹H NMR (400 MHz, DMSO-d6) δ: 8.16 (s, 1H), 7.33-7.30 (m, 2H), 7.23 (s, 1H), 7.16-7.11 (m, 3H), 5.44 (t, J = 5.8 Hz, 1H), 4.87 (s, 2H), 4.42 (d, J = 5.8 Hz, 2H), 2.37 (s, 3H). | |
| | | Exchangeable —NH₂ protons were not observed. | |
| | | LCMS Method C: m/z 482 (M+H)⁺ (ES+), at 1.97 min, UV active | |
| 1-60 | 5-amino-8-[2-(hydroxymethyl)-6-methoxy-4-pyridyl]-2-[(5-methyloxazol-4-yl)methyl]-7-phenyl-[1,2,4]triazolo[4,3-c]pyrimidin-3-one | Synthetic route q | 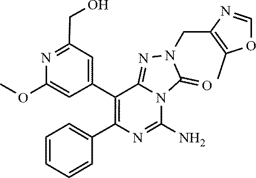 [SMILES: COc1cc(-c2c(-c3ccccc3)nc(N)n3c(=O)n(Cc4ncoc4C)nc23)cc(CO)n1] |
| | | Intermediates 1 & 21 (step 1), 46 (step 2) | |
| | | ¹H NMR (400 MHz, DMSO-d6) δ: 8.15 (s, 1H), 7.28-7.24 (m, 5H), 6.86 (s, 1H), 6.42 (s, 1H), 5.23 (t, J = 5.6 Hz, 1H), 4.87 (s, 2H), 4.37 (d, J = 5.60 Hz, 2H), 3.75 (s, 3H), 2.35 (s, 3H). | |
| | | Exchangeable —NH₂ Protons were not observed. | |
| | | LCMS Method A: m/z 460 (M+H)⁺ (ES+), at 2.08 min, UV active | |
| 1-61 | 5-amino-8-[2-(hydroxymethyl)-6-methoxy-4-pyridyl]-2-[(1-methylimidazol-2-yl)methyl]-7-phenyl-[1,2,4]triazolo[4,3-c]pyrimidin-3-one | Synthetic route v | 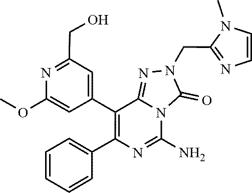 [SMILES: COc1cc(-c2c(-c3ccccc3)nc(N)n3c(=O)n(Cc4nccn4C)nc23)cc(CO)n1] |
| | | Intermediates 1 & 11 (step 1), 46 (step 2) | |
| | | ¹H NMR (400 MHz, DMSO-d6) δ: 7.29-7.27 (m, 7H), 7.17 (s, 1H), 6.87 (s, 2H), 6.40 (s, 1H), 5.23 (s, 1H), 5.12 (s, 2H), 4.37 (d, J = 2.8 Hz, 2H), 3.75 (s, 3H), 3.71 (s, 3H). | |
| | | LCMS Method C: m/z 459 (M+H)⁺ (ES+), at 1.16 min, UV active | |
| 1-62 | 5-amino-8-[2-(hydroxymethyl)-6-methoxy-4-pyridyl]-2-[(1-methylimidazol-2-yl)methyl]-7-(2,3,4,5,6-pentadeuteriophenyl)-[1,2,4]triazolo[4,3-c]pyrimidin-3-one | Synthetic route v | 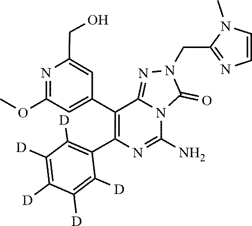 [SMILES: [2H]c1c([2H])c([2H])c(-c2nc(N)n3c(=O)n(Cc4nccn4C)nc3c2-c2cc(CO)nc(OC)c2)c([2H])c1[2H]] |
| | | Intermediates 39 & 11 (step 1), 46 (step 2) | |
| | | ¹H NMR (400 MHz, DMSO-d6) δ: 7.11 (s, 1H), 6.87 (s, 1H), 6.77 (s, 1H), 6.40 (s, 1H), 5.23 (t, J = 5.6 Hz, 1H), 5.07 (s, 2H), 4.37 (d, J = 5.6 Hz, 2H), 3.75 (s, 3H), 3.69 (s, 3H). | |
| | | Exchangeable —NH₂ protons were not observed. | |
| | | LCMS Method C: m/z 464 (M+H)⁺ (ES+), at 1.29 min, UV active | |
| 1-63 | 5-amino-8-[2-(hydroxymethyl)-6-methoxy-4-pyridyl]-2-[(5-methyloxazol-4-yl)methyl]-7-(2,3,4,5,6-pentadeuteriophenyl)-[1,2,4]triazolo[4,3-c]pyrimidin-3-one | Synthetic route v | 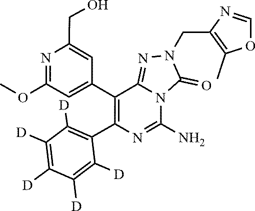 [SMILES: [2H]c1c([2H])c([2H])c(-c2nc(N)n3c(=O)n(Cc4ncoc4C)nc3c2-c2cc(CO)nc(OC)c2)c([2H])c1[2H]] |
| | | Intermediates 39 & 21 (step 1), 46 (step 2) | |
| | | ¹H NMR (400 MHz, DMSO-d6) δ: 8.15 (s, 1H), 6.86 (s, 1H), 6.42 (s, 1H), 5.24 (t, J = 5.8 Hz, 1H), 4.87 (s, 2H), 4.37 (d, J = 5.8 Hz, 2H), 3.75 (s, 3H), 2.34 (s, 3H). | |
| | | Exchangeable —NH₂ Protons were not observed. | |
| | | LCMS Method C: m/z 465 (M+H)⁺ (ES+), at 1.65 min, UV active | |
| 1-64 | 5-amino-7-(4-fluorophenyl)-8-[2-(hydroxymethyl)-6-methoxy-4-pyridyl]-2-[(5-methyloxazol-4-yl)methyl]-[1,2,4]triazolo[4,3-c]pyrimidin-3-one | Synthetic route q | 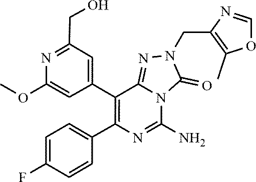 [SMILES: COc1cc(-c2c(-c3ccc(F)cc3)nc(N)n3c(=O)n(Cc4ncoc4C)nc23)cc(CO)n1] |
| | | Intermediates 26 & 21 (step 1), 46 (step 2) | |
| | | ¹H NMR (400 MHz, DMSO-d6) δ: 8.15 (s, 1H), 7.36-7.30 (m, 2H), 7.16-7.08 (m, 2H), 6.82 (s, 1H), 6.42 (s, 1H), 5.24 (t, J = 5.7 Hz, 1H), 4.86 (s, 2H), 4.37 (d, J = 5.7 Hz, 2H), 3.77 (s, 3H), 2.34 (s, 3H). | |
| | | Exchangeable —NH₂ protons were not observed. | |
| | | LCMS Method C: m/z 478 (M+H)⁺ (ES+), at 1.93 min, UV active | |
| 1-65 | 5-amino-7-(4-fluorophenyl)-8-[2-(hydroxymethyl)-6-methoxy-4-pyridyl]-2-[(1-methylimidazol-2-yl)methyl]-[1,2,4]triazolo[4,3-c]pyrimidin-3-one | Synthetic route v | 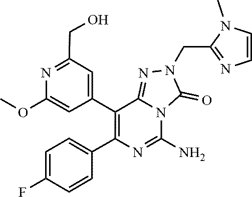 [SMILES: COc1cc(-c2c(-c3ccc(F)cc3)nc(N)n3c(=O)n(Cc4nccn4C)nc23)cc(CO)n1] |
| | | Intermediates 26 & 11 (step 1), 46 (step 2) | |
| | | ¹H NMR (400 MHz, DMSO-d6) δ: 7.34-7.30 (m, 2H), 7.12-7.08 (m, 3H), 6.71-6.68 (m, 2H), 6.44 (s, 1H), 5.22 (s, 1H), 5.05 (s, 2H), 4.36 (s, 2H), 3.75 (s, 3H), 3.67 (s, 3H). | |
| | | Exchangeable —NH₂ protons were not observed. | |
| | | LCMS Method C: m/z 477 (M+H)⁺ (ES+), at 1.22 min, UV active | |
| 1-66 | 5-amino-8-[2-(hydroxymethyl)-6-(trifluoromethyl)-4-pyridyl]-2-[(5-methyloxazol-4-yl)methyl]-7-phenyl-[1,2,4]triazolo[4,3-c]pyrimidin-3-one | Synthetic route q | 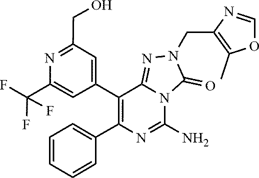 [SMILES: Cc1ocnc1Cn1nc2c(-c3cc(CO)nc(C(F)(F)F)c3)c(-c3ccccc3)nc(N)n2c1=O] |
| | | Intermediates 1 & 21 (step 1), 48 (step 2) | |
| | | ¹H NMR (400 MHz, DMSO-d6) δ: 8.16 (s, 1H), 7.64 (s, 1H), 7.32-7.27 (m, 6H), 5.52 (d, J = 6.1 Hz, 1H), 4.87 (s, 2H), 4.52 (d, J = 6.1 Hz, 2H), 2.33 (s, 3H). | |
| | | Exchangeable —NH₂ protons were not observed. | |
| | | LCMS Method C: m/z 498 (M+H)⁺ (ES+), at 2.11 min, UV active | |
| 1-67 | 5-amino-8-[2-(hydroxymethyl)-6-(trifluoromethyl)-4-pyridyl]-2-[(1-methylimidazol-2-yl)methyl]-7-phenyl-[1,2,4]triazolo[4,3-c]pyrimidin-3-one | Synthetic route q | 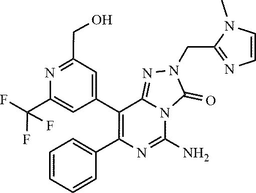 [SMILES: Cn1ccnc1Cn1nc2c(-c3cc(CO)nc(C(F)(F)F)c3)c(-c3ccccc3)nc(N)n2c1=O] |
| | | Intermediates 1 & 11 (step 1), 48 (step 2) | |
| | | ¹H NMR (400 MHz, DMSO-d6) δ: 7.65 (s, 1H), 7.34 - 7.25 (m, 7H), 6.97 (s, 1H), 5.51 (t, J = 5.6 Hz, 1H), 5.16 (s, 2H), 4.52 (d, J = 5.6 Hz, 2H), 3.75 (s, 3H). | |
| | | Exchangeable —NH₂ protons were not observed. | |
| | | LCMS Method C: m/z 497 (M+H)⁺ (ES+), at 1.31 min, UV active | |
| 2-1 | tert-butyl 3-[5-amino-8-(2,6-dimethyl-4-pyridyl)-3-oxo-7-phenyl-[1,2,4]triazolo[4,3-c]pyrimidin-2-yl]pyrrolidine-1-carboxylate | Synthetic route a | 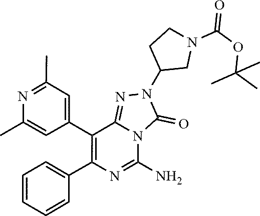 [SMILES: Cc1cc(-c2c(-c3ccccc3)nc(N)n3c(=O)n(C4CCN(C(=O)OC(C)(C)C)C4)nc23)cc(C)n1] |
| | | Intermediates 1 & 50 (step 1) | |
| | | ¹H NMR (400 MHz, DMSO-d6) δ 8.36 (br s, 1H), 7.76 (br s, 1H), 7.32 - 7.26 (m, 5H), 6.80 (s, 2H), 4.82 (t, J = 4.4 Hz, 1H), 3.61 - 3.48 (m, 4H), 2.28 (s, 6H), 2.21 - 2.14 (m, 2H), 1.35 (s, 9H). | |
| | | LCMS Method A: m/z 502 (M+H)⁺ (ES+), at 3.42 min, UV active | |
| 2-2 | 5-amino-8-(2,6-dimethyl-4-pyridyl)-7-phenyl-2-pyrrolidin-3-yl-[1,2,4]triazolo[4,3-c]pyrimidin-3-one | Synthetic route w | 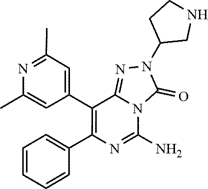 [SMILES: Cc1cc(-c2c(-c3ccccc3)nc(N)n3c(=O)n(C4CCNC4)nc23)cc(C)n1] |
| | | ¹H NMR (400 MHz, CD₃OD) δ: 7.34 -7.25 (m, 5H), 6.96 (s, 2H), 4.91 (d, J = 13.6 Hz, 1H), 3.34 - 3.32 (m, 3H), 3.21 - 3.15 (m, 1H), 2.37 (s, 6H), 2.28 - 2.12 (m, 2H). | |
| | | Exchangeable —NH,—NH₂ protons were not observed | |
| | | LCMS Method A: m/z 402 (M+H)⁺ (ES+), at 1.95 min, UV active | |
| 2-3 | 5-amino-8-(2,6-dimethyl-4-pyridyl)-2-(1-methylpyrrolidin-3-yl)-7-phenyl-[1,2,4]triazolo[4,3-c]pyrimidin-3-one | Synthetic route a | 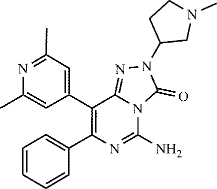 |
| | | Intermediates 1 & 51 (step 1) | |
| | | ¹H NMR (400 MHz, DMSO-d6) δ: 7.29 - 7.25 (m, 5H), 6.84 (s, 2H), 4.77 - 4.75 (m, 1H), 2.95 (t, J = 9.6 Hz, 1H), 2.68 - 2.55 (m, 3H), 2.29 (s, 6H), 2.26 (s, 3H), 2.21 - 2.13 (m, 2H). | |
| | | Exchangeable —NH₂ protons were not observed | |
| | | LCMS Method A: m/z 416 (M+H)⁺ (ES+), at 2.00 min, UV active | |
| 2-4 | tert-butyl 2-[[5-amino-8-(2,6-dimethyl-4-pyridyl)-3-oxo-7-phenyl-[1,2,4]triazolo[4,3-c]pyrimidin-2-yl]methyl]pyrrolidine-1-carboxylate | Synthetic route a | 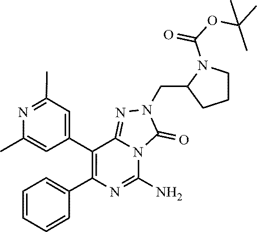 [SMILES: Cc1cc(-c2c(-c3ccccc3)nc(N)n3c(=O)n(CC4CCCN4C(=O)OC(C)(C)C)nc23)cc(C)n1] |
| | | Intermediates 1 & 52 (step 1) | |
| | | ¹H NMR (400 MHz, DMSO-d6) δ: 8.27 - 7.89 (m, 2H), 7.28 - 7.25 (m, 5H), 6.82 (s, 2H), 4.14 - 4.05 (m, 1H), 3.83 - 3.82 (m, 2H), 3.32 - 3.27 (m, 2H), 2.33 (s, 6H), 1.83 (br s, 4H), 1.31 (s, 9H). | |
| | | LCMS Method A: m/z 516 (M+H)⁺ (ES+), at 3.35 min, UV active | |
| 2-5 | 5-amino-8-(2,6-dimethyl-4-pyridyl)-7-phenyl-2-(pyrrolidin-2-ylmethyl)-[1,2,4]triazolo[4,3-c]pyrimidin-3-one | Synthetic route w | 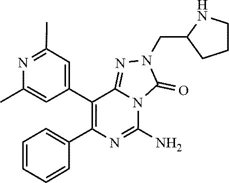 [SMILES: Cc1cc(-c2c(-c3ccccc3)nc(N)n3c(=O)n(CC4CCCN4)nc23)cc(C)n1] |
| | | Example 2-4 | |
| | | ¹H NMR (400 MHz, DMSO-d6) δ: 8.16 - 7.69 (m, 2H), 7.27 - 7.25 (m, 5H), 6.83 (s, 2H), 3.77 - 3.67 (m, 2H), 3.42 - 3.39 (m, 1H), 2.85 - 2.77 (m, 2H), 2.29 (s, 6H), 1.80 - 1.64 (m, 3H), 1.48 - 1.42 (m, 1H). | |
| | | Exchangeable —NH proton was not observed. | |
| | | LCMS Method A: m/z 416 (M+H)⁺ (ES+), at 1.90 min, UV active | |
| 2-6 | 5-amino-8-(2,6-dimethyl-4-pyridyl)-2-[(1-methylpyrrolidin-2-yl)methyl]-7-phenyl-[1,2,4]triazolo[4,3-c]pyrimidin-3-one | Synthetic route x | 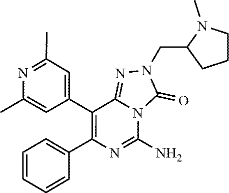 [SMILES: Cc1cc(-c2c(-c3ccccc3)nc(N)n3c(=O)n(CC4CCCN4C)nc23)cc(C)n1] |
| | | ¹H NMR (400 MHz, DMSO-d6) δ: 8.23 - 7.98 (m, 2H), 7.27 - 7.26 (m, 5H), 6.82 (s, 2H), 3.87 - 3.82 (m, 1H), 3.72 - 3.67 (m, 1H), 2.95 - 2.94 (m, 2H), 2.29 (s, 9H), 2.18 - 2.16 (m, 1H), 1.86 - 1.81 (m, 1H), 1.71 - 1.61 (m, 3H). | |
| | | LCMS Method A: m/z 430 (M+H)⁺ (ES+), at 1.91 min, UV active | |
| 2-7 | 5-amino-8-(2,6-dimethyl-4-pyridyl)-2-[[(2R)-1-methylpyrrolidin-2-yl]methyl]-7-phenyl-[1,2,4]triazolo[4,3-c]pyrimidin-3-one | Synthetic route e (purified by prep HPLC method C) | 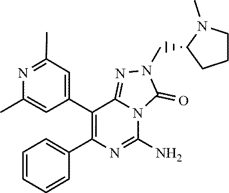 [SMILES: Cc1cc(-c2c(-c3ccccc3)nc(N)n3c(=O)n(C[C@H]4CCCN4C)nc23)cc(C)n1] |
| | | Intermediates 6 & 53 | |
| | | ¹H NMR (400 MHz, DMSO-d6) δ: 8.23 - 7.62 (m, 2H), 7.28 - 7.26 (m, 5H), 6.82 (s, 2H), 3.87 - 3.82 (m, 1H), 3.71 - 3.67 (m, 1H), 2.95 - 2.94 (m, 1H), 2.51 - 2.48 (m, 1H), 2.29 (s, 9H), 2.20 - 2.16 (m, 1H), 1.83 - 1.81 (m, 1H), 1.71 - 1.61 (m, 3H). | |
| | | LCMS Method A: m/z 430 (M+H)⁺ (ES+), at 1.94 min, UV active | |
| 2-8 | 5-amino-8-(2,6-dimethyl-4-pyridyl)-2-[[(2S)-1-methylpyrrolidin-2-yl]methyl]-7-phenyl-[1,2,4]triazolo[4,3-c]pyrimidin-3-one | Synthetic route e (purified by prep HPLC method C) | 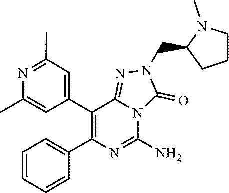 [SMILES: Cc1cc(-c2c(-c3ccccc3)nc(N)n3c(=O)n(C[C@@H]4CCCN4C)nc23)cc(C)n1] |
| | | Intermediates 6 & 54 | |
| | | ¹H NMR (400 MHz, DMSO-d6) δ: 8.23 - 7.71 (m, 2H), 7.28 - 7.26 (m, 5H), 6.81 (s, 2H), 3.85 - 3.82 (m, 1H), 3.71 ― 3.67 (m, 1H), 2.95 - 2.94 (m, 1H), 2.50 - 2.45 (m, 1H), 2.28 (s, 9H), 2.16 - 2.13 (m, 1H), 1.83 - 1.81 (m, 1H), 1.70 - 1.62 (m, 3H). | |
| | | LCMS Method A: m/z 430 (M+H)⁺ (ES+), at 1.94 min, UV active | |
| 2-9 | 5-amino-8-(2,6-dimethyl-4-pyridyl)-2-[[(2S)-1-(2-methoxyethyl)pyrrolidin-2-yl]methyl]-7-phenyl-[1,2,4]triazolo[4,3-c]pyrimidin-3-one | Synthetic route a | 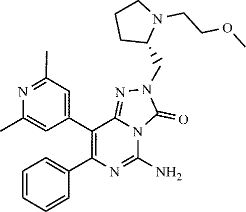 |
| | | Intermediates 1 & 55 (step 1) | |
| | | ¹H NMR (400 MHz, DMSO-d6) δ: 8.15 (s, 2H), 7.29 - 7.25 (m, 5H), 6.81 (s, 2H), 3.79 - 3.74 (m, 1H), 3.66 - 3.65 (m, 1H), 3.37 - 3.36 (m, 2H), 3.19 (s, 3H), 3.02 - 3.01 (m, 1H), 2.87 - 2.82 (m, 2H), 2.46 - 2.44 (m, 1H), 2.28 - 2.24 (m, 7H), 1.79 - 1.75 (m, 1H), 1.66 - 1.62 (m, 3H). | |
| | | LCMS Method A: m/z 474 (M+H)⁺ (ES+), at 2.04 min, UV active | |
| 2-10 | 5-amino-8-(2,6-dimethyl-4-pyridyl)-2-[[(2R)-1-(2-methoxyethyl)pyrrolidin-2-yl]methyl]-7-phenyl-[1,2,4]triazolo[4,3-c]pyrimidin-3-one | Synthetic route a | 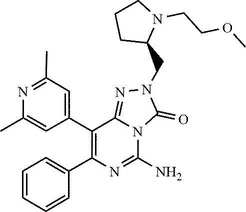 |
| | | Intermediates 1 & 57 (step 1) | |
| | | ¹H NMR (400 MHz, DMSO-d6) δ: 8.15 (s, 2H), 7.27 - 7.24 (m, 5H), 6.82 (s, 2H), 3.79 - 3.76 (m, 1H), 3.68 - 3.66 (m, 1H), 3.40 - 3.37 (m, 2H), 3.20 (s, 3H), 3.03 - 3.02 (m, 1H), 2.88 - 2.84 (m, 2H), 2.46 - 2.44 (m, 2H), 2.29 - 2.24 (m, 7H), 1.79 - 1.77 (m, 1H), 1.66 - 1.64 (m, 3H). | |
| | | LCMS Method A: m/z 474 (M+H)⁺ (ES+), at 2.05 min, UV active | |
| 2-11 | 5-amino-2-[[(2S)-4,4-difluoropyrrolidin-2-yl]methyl]-8-(2,6-dimethyl-4-pyridyl)-7-phenyl-[1,2,4]triazolo[4,3-c]pyrimidin-3-one | Synthetic route y | 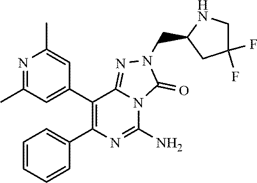 [SMILES: Cc1cc(-c2c(-c3ccccc3)nc(N)n3c(=O)n(C[C@@H]4CC(F)(F)CN4)nc23)cc(C)n1] |
| | | ¹H NMR (400 MHz, DMSO-d6) δ: 7.90 (s, 2H), 7.30 - 7.25 (m, 5H), 6.82 (s, 2H), 3.88 - 3.76 (m, 2H), 3.58 - 3.57 (m, 1H), 3.18 - 3.06 (m, 3H), 2.39 - 2.33 (m, 1H), 2.29 (s, 6H), 2.12 - 2.02 (m, 1H). | |
| | | LCMS Method A: m/z 452 (M+H)⁺ (ES+), at 2.04 min, UV active | |
| 2-12 | 5-amino-2-[[(2R)-4,4-difluoropyrrolidin-2-yl]methyl]-8-(2,6-dimethyl-4-pyridyl)-7-phenyl-[1,2,4]triazolo[4,3-c]pyrimidin-3-one | Synthetic route y | 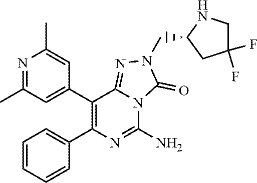 [SMILES: Cc1cc(-c2c(-c3ccccc3)nc(N)n3c(=O)n(C[C@H]4CC(F)(F)CN4)nc23)cc(C)n1] |
| | | Intermediates 60 (step 1) | |
| | | ¹H NMR (400 MHz, DMSO-d6) δ: 8.30 - 7.70 (m, 2H), 7.27 - 7.26 (m, 5H), 6.82 (s, 2H), 3.86 - 3.81 (m, 2H), 3.61 - 3.58 (m, 1H), 3.21 - 3.06 (m, 2H), 2.30 - 2.29 (m, 1H), 2.29 (s, 6H), 2.12 - 2.02 (m, 1H). | |
| | | Exchangeable —NH proton was not observed. | |
| | | LCMS Method A: m/z 452 (M+H)⁺ (ES+), at 2.03 min, UV active | |
| 2-13 | 5-amino-2-[[(2S)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one | Synthetic route x (purified by prep HPLC method C) | 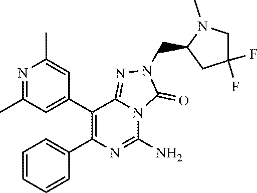 [SMILES: Cc1cc(-c2c(-c3ccccc3)nc(N)n3c(=O)n(C[C@@H]4CC(F)(F)CN4C)nc23)cc(C)n1] |
| | | Example 2-11 | |
| | | ¹H NMR (400 MHz, DMSO-d6) δ: 8.40 - 7.70 (m, 2H), 7.28 - 7.26 (m, 5H), 6.81 (s, 2H), 3.97 - 3.85 (m, 2H), 3.33 - 3.32 (m, 1H), 2.91 - 2.86 (m, 1H), 2.69 - 2.61 (m, 2H), 2.35 - 2.34 (m, 1H), 2.33 (s, 3H), 2.29 (s, 6H). | |
| | | LCMS Method E: m/z 466 (M+H)⁺ (ES+), at 5.29 min, UV active | |
| 2-14 | 5-amino-2-[[(2R)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one | Synthetic route x (purified by prep HPLC method C) | 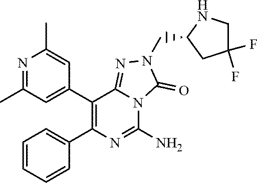 [SMILES: Cc1cc(-c2c(-c3ccccc3)nc(N)n3c(=O)n(C[C@H]4CC(F)(F)CN4)nc23)cc(C)n1] |
| | | Example 2-12 | |
| | | ¹H NMR (400 MHz, DMSO-d6) δ: 8.40 - 7.70 (m, 2H), 7.28 - 7.26 (m, 5H), 6.81 (s, 2H), 3.97 - 3.85 (m, 2H), 3.33 - 3.32 (m, 1H), 2.91 - 2.86 (m, 1H), 2.69 - 2.61 (m, 2H), 2.35 - 2.34 (m, 1H), 2.33 (s, 3H), 2.29 (s, 6H). | |
| | | LCMS Method E: m/z 466 (M+H)⁺ (ES+), at 5.29 min, UV active | |
| 2-15 | 5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one | Synthetic route z | 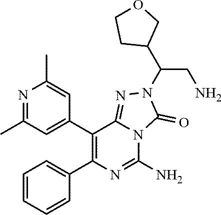 [SMILES: Cc1cc(-c2c(-c3ccccc3)nc(N)n3c(=O)n(C(CN)C4CCOC4)nc23)cc(C)n1] |
| | | ¹H NMR (400 MHz, Chloroform-d) δ: 7.33 ― 7.18 (m, 5H), 6.79 (d, J = 4.4 Hz, 2H), 4.21 ― 3.42 (m, 5H), 3.20 ― 2.47 (m, 3H), 2.42 - 2.38 (m, 6H), 2.18 - 1.21 (m, 6H). | |
| | | LCMS Method F: m/z 446 (M+H)⁺ (ES+), at 0.80 min, UV active | |
| 2-16 | 5-amino-8-(2,6-dimethyl-4-pyridyl)-7-phenyl-2-[[(2R)-tetrahydrofuran-2-yl]methyl]-[1,2,4]triazolo[4,3-c]pyrimidin-3-one | Synthetic route e | 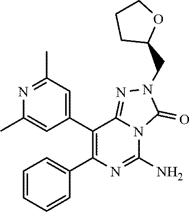 [SMILES: Cc1cc(-c2c(-c3ccccc3)nc(N)n3c(=O)n(C[C@H]4CCCO4)nc23)cc(C)n1] |
| | | Intermediates 6 & 62 | |
| | | ¹H NMR (400 MHz, DMSO-d6) δ: 7.28 - 7.27 (m, 5H), 6.84 (s, 2H), 4.14 (t, J = 4.5 Hz, 1H), 3.87 - 3.84 (m, 1H), 3.73 - 3.63 (m, 2H), 3.62 (t, J = 2.4 Hz, 1H), 2.32 (s, 6H), 1.96-1.94 (m, 1H), 1.91-1.86 (m, 2H), 1.85-1.83 (m, 1H). | |
| | | Exchangeable —NH₂ protons were not observed. | |
| | | LCMS Method A: m/z 417 (M+H)⁺ (ES+), at 2.38 min, UV active | |
| 2-17 | 5-amino-8-(2,6-dimethyl-4-pyridyl)-7-phenyl-2-[[(2S)-tetrahydrofuran-2-yl]methyl]-[1,2,4]triazolo[4,3-c]pyrimidin-3-one | Synthetic route e | 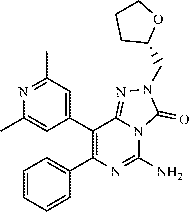 [SMILES: Cc1cc(-c2c(-c3ccccc3)nc(N)n3c(=O)n(C[C@@H]4CCCO4)nc23)cc(C)n1] |
| | | Intermediates 6 & 63 | |
| | | ¹H NMR (400 MHz, DMSO-d6) δ: 7.28 - 7.27 (m, 5H), 6.84 (s, 2H), 4.14 (t, J = 4.5 Hz, 1H), 3.87 - 3.84 (m, 1H), 3.73 - 3.63 (m, 2H), 3.62 (t, J = 2.4 Hz, 1H), 2.32 (s, 6H), 1.96-1.94 (m, 1H), 1.91-1.86 (m,2H), 1.85-1.83 (m, 1H). | |
| | | Exchangeable —NH₂ protons were not observed. | |
| | | LCMS Method A: m/z 417 (M+H)⁺ (ES+), at 2.43 min, UV active | |
| 2-18 | 5-amino-8-(2,6-dimethyl-4-pyridyl)-2-[[(2R,4S)-4-fluoropyrrolidin-2-yl]methyl]-7-phenyl-[1,2,4]triazolo[4,3-c]pyrimidin-3-one | Synthetic route aa | 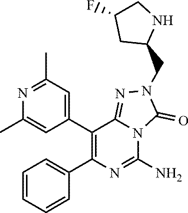 [SMILES: Cc1cc(-c2c(-c3ccccc3)nc(N)n3c(=O)n(C[C@H]4C[C@H](F)CN4)nc23)cc(C)n1] |
| | | ¹H NMR (400 MHz, DMSO-d6) δ: 8.32 - 7.44 (m, 2H), 7.30 - 7.26 (m, 5H), 6.78 (s, 2H), 5.34 - 5.10 (m, 1H), 3.82 - 3.77 (m, 1H), 3.71 - 3.58 (m, 2H), 3.10 - 2.91 (m, 3H), 2.30 (s, 6H), 2.12 - 2.00 (m, 1H), 1.76 - 1.60 (m, 1H). | |
| | | LCMS Method A: m/z 434 (M+H)⁺ (ES+), at 1.91 min, UV active | |
| 2-19 | 5-amino-8-(2,6-dimethyl-4-pyridyl)-2-[[(2R,4R)-4-fluoropyrrolidin-2-yl]methyl]-7-phenyl-[1,2,4]triazolo[4,3-c]pyrimidin-3-one | Synthetic route aa | 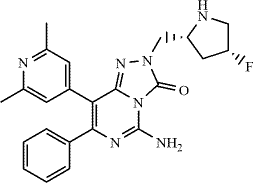 [SMILES: Cc1cc(-c2c(-c3ccccc3)nc(N)n3c(=O)n(C[C@H]4C[C@@H](F)CN4)nc23)cc(C)n1] |
| | | Intermediates 64 (Step 1) | |
| | | ¹H NMR (400 MHz, DMSO-d6) δ: 8.32 - 7.44 (m, 2H), 7.30 - 7.26 (m, 5H), 6.78 (s, 2H), 5.34 - 5.10 (m, 1H), 3.82 - 3.77 (m, 1H), 3.71 - 3.58 (m, 2H), 3.10 - 2.91 (m, 3H), 2.30 (s, 6H), 2.12 - 2.00 (m, 1H), 1.76 - 1.60 (m, 1H). | |
| | | LCMS Method A: m/z 434 (M+H)⁺ (ES+), at 1.89 min, UV active | |
| 2-20 | 5-amino-8-(2,6-dimethyl-4-pyridyl)-2-[[(2S,4S)-4-fluoropyrrolidin-2-yl]methyl]-7-phenyl-[1,2,4]triazolo[4,3-c]pyrimidin-3-one | Synthetic route aa | 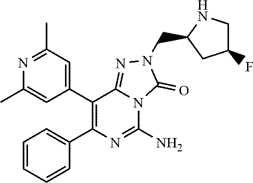 [SMILES: Cc1cc(-c2c(-c3ccccc3)nc(N)n3c(=O)n(C[C@@H]4C[C@H](F)CN4)nc23)cc(C)n1] |
| | | Intermediates 59 (step 1) | |
| | | ¹H NMR (400 MHz, DMSO-d6) δ: 8.32 - 7.44 (m, 2H), 7.30 - 7.26 (m, 5H), 6.78 (s, 2H), 5.34 - 5.10 (m, 1H), 3.82 - 3.77 (m, 1H), 3.71 - 3.58 (m, 2H), 3.10 - 2.91 (m, 3H), 2.30 (s, 6H), 2.12 - 2.00 (m, 1H), 1.76 - 1.60 (m, 1H). | |
| | | LCMS Method A: m/z 434 (M+H)⁺ (ES+), at 1.89 min, UV active | |
| 2-21 | 5-amino-8-(2,6-dimethyl-4-pyridyl)-2-[[(2S,4R)-4-fluoropyrrolidin-2-yl]methyl]-7-phenyl-[1,2,4]triazolo[4,3-c]pyrimidin-3-one | Synthetic route aa | 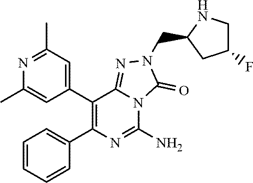 [SMILES: Cc1cc(-c2c(-c3ccccc3)nc(N)n3c(=O)n(C[C@@H]4C[C@@H](F)CN4)nc23)cc(C)n1] |
| | | Intermediates 65 (step 1) | |
| | | ¹H NMR (400 MHz, DMSO-d6) δ: 8.32 - 7.44 (m, 2H), 7.30 - 7.26 (m, 5H), 6.78 (s, 2H), 5.34 - 5.10 (m, 1H), 3.82 - 3.77 (m, 1H), 3.71 - 3.58 (m, 2H), 3.10 - 2.91 (m, 3H), 2.30 (s, 6H), 2.12 - 2.00 (m, 1H), 1.76 - 1.60 (m, 1H). | |
| | | LCMS Method A: m/z 434 (M+H)⁺ (ES+), at 1.90 min, UV active | |
| 2-22 | 5-amino-8-(2,6-dimethyl-4-pyridyl)-2-[[(2S,4S)-4-hydroxypyrrolidin-2-yl]methyl]-7-phenyl-[1,2,4]triazolo[4,3-c]pyrimidin-3-one | Synthetic route ab | 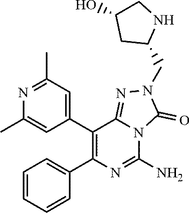 [SMILES: Cc1cc(-c2c(-c3ccccc3)nc(N)n3c(=O)n(C[C@@H]4C[C@H](O)CN4)nc23)cc(C)n1] |
| | | ¹H NMR (400 MHz, DMSO-d6) δ: 8.27 - 7.85 (m, 2H), 7.28 - 7.25 (m, 5H), 6.83 (s, 2H), 4.71 (d, J = 4.0 Hz, 1H), 4.16 (br s, 1H), 3.93 - 3.88 (m, 1H), 3.78 - 3.73 (m, 1H), 3.41 - 3.38 (m, 1H), 2.84 - 2.80 (m, 1H), 2.69 - 2.66 (m, 2H), 2.29 (s, 6H), 2.04 - 1.97 (m, 1H), 1.41 - 1.35 (m, 1H). | |
| | | LCMS Method A: m/z 432 (M+H)⁺ (ES+), at 1.81 min, UV active | |
| 2-23 | 5-amino-8-(2,6-dimethyl-4-pyridyl)-2-[[(2R,4R)-4-hydroxypyrrolidin-2-yl]methyl]-7-phenyl-[1,2,4]triazolo[4,3-c]pyrimidin-3-one | Synthetic route ab (final step purification by SCX cartridge) | 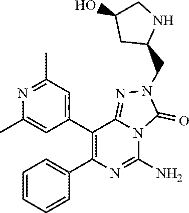 [SMILES: Cc1cc(-c2c(-c3ccccc3)nc(N)n3c(=O)n(C[C@H]4C[C@@H](O)CN4)nc23)cc(C)n1] |
| | | Intermediates 60 (step 1) | |
| | | ¹H NMR (400 MHz, DMSO-d6) δ: 8.27 - 7.85 (m, 2H), 7.28 - 7.25 (m, 5H), 6.83 (s, 2H), 4.71 (d, J = 4.0 Hz, 1H), 4.16 (br s, 1H), 3.93 - 3.88 (m, 1H), 3.78 - 3.73 (m, 1H), 3.41 - 3.38 (m, 1H), 2.84 - 2.80 (m, 1H), 2.69 - 2.66 (m, 2H), 2.29 (s, 6H), 2.04 - 1.97 (m, 1H), 1.41 - 1.35 (m, 1H). | |
| | | LCMS Method A: m/z 432 (M+H)⁺ (ES+), at 1.82 min, UV active | |
| 2-24 | 5-amino-8-(2,6-dimethyl-4-pyridyl)-2-[[(2R,4R)-4-hydroxy-1-methyl-pyrrolidin-2-yl]methyl]-7-phenyl-[1,2,4]triazolo[4,3-c]pyrimidin-3-one | Synthetic route x (purified by prep HPLC method A) | 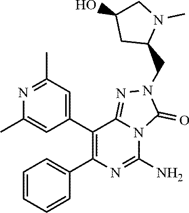 |
| | | Example 2-23 | |
| | | ¹H NMR (400 MHz, DMSO-d6) δ: 7.34 - 7.25 (m, 5H), 6.97 (s, 2H), 4.29 (s, 1H), 4.07 - 4.05 (m, 2H), 3.08 (d, J = 9.0 Hz, 1H), 2.84 - 2.83 (m, 1H), 2.56 - 2.38 (m, 11H), 1.89 - 1.80 (m, 1H). | |
| | | Note: Exchangeable —NH₂ and —OH protons were not observed. | |
| | | LCMS Method A: m/z 446 (M+H)⁺ (ES+), at 1.83 min, UV active | |
| 2-25 | 5-amino-8-(2,6-dimethyl-4-pyridyl)-2-[[(2S,4S)-4-hydroxy-1-methyl-pyrrolidin-2-yl]methyl]-7-phenyl-[1,2,4]triazolo[4,3-c]pyrimidin-3-one | Synthetic route x (purified by prep HPLC method A) | 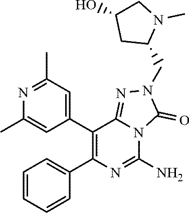 |
| | | Example 2-22 | |
| | | ¹H NMR (400 MHz, DMSO-d6) δ: 7.33 - 7.24 (m, 5H), 6.97 (s, 2H), 4.30 (s, 1H), 4.10 - 4.09 (m, 2H), 3.14 - 3.13 (m, 1H), 3.03 - 2.90 (m, 1H), 2.63 - 2.37 (m, 11H), 1.98 - 1.88 (m, 1H). | |
| | | Exchangeable —NH₂, —OH protons were not observed. | |
| | | LCMS Method A: m/z 446 (M+H)⁺ (ES+), at 1.82 min, UV active | |
| 2-26 | 5-amino-8-(2,6-dimethyl-4-pyridyl)-7-phenyl-2-(2-pyrrolidin-1-ylethyl)-[1,2,4]triazolo[4,3-c]pyrimidin-3-one | Synthetic route a | 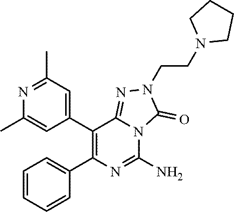 [SMILES: Cc1cc(-c2c(-c3ccccc3)nc(N)n3c(=O)n(CCN4CCCC4)nc23)cc(C)n1] |
| | | Intermediates 1 & 66 (step 1) | |
| | | ¹H NMR (400 MHz, DMSO-d6) δ 7.34 - 7.24 (m, 5H), 6.95 (s, 2H), 4.18 ― 4.04 (m, 2H), 2.96 (br s, 2H), 2.72 (brs, 4H), 2.37 (s, 6H), 1.85 (br s, 4H). | |
| | | Exchangeable —NH₂ protons were not observed. | |
| | | LCMS Method A: m/z 430 (M+H)⁺ (ES+), at 2.00 min, UV active | |
| 2-27 | 5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3one | Synthetic route ac | 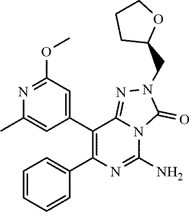 [SMILES: COc1cc(-c2c(-c3ccccc3)nc(N)n3c(=O)n(C[C@H]4CCCO4)nc23)cc(C)n1] |
| | | ¹H NMR (400 MHz, DMSO-d6) δ: 7.29 (s, 5H), 6.57 (s, 1H), 6.45 (s, 1H), 4.15-4.12 (m, 1H), 3.89-3.85 (m, 1H), 3.83 (s, 5H), 3.74-3.62 (m, 1H), 2.25 (s, 3H), 1.97 (s, 3H), 1.95-1.92 (m, 1H). | |
| | | Exchangeable —NH₂ Protons were not observed. | |
| | | LCMS Method C: m/z 433 (M+H)⁺ (ES+), at 1.99 min, UV active | |
| 2-28 | 5-amino-8-[2-(hydroxymethyl)-6-methyl-4-pyridyl]-7-phenyl-2-[[(2S)-tetrahydrofuran-2-yl]methyl]-[1,2,4]triazolo[4,3-c]pyrimidin-3-one | Synthetic route q and SFC Method A | 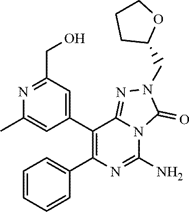 [SMILES: Cc1cc(-c2c(-c3ccccc3)nc(N)n3c(=O)n(C[C@@H]4CCCO4)nc23)cc(CO)n1] |
| | | ¹H NMR (500 MHz, DMSO-d6) δ: 7.33 - 7.22 (m, 5H), 7.13 (s, 1H), 6.84 (s, 1H), 5.32 - 5.19 (m, 1H), 4.43 (s, 2H), 4.19 - 4.11 (m, 1H), 3.89 - 3.81 (m, 1H), 3.74 (br d, 2H), 3.65 - 3.58 (m, 1H), 2.30 (s, 3H), 1.99 - 1.91 (m, 1H), 1.89 - 1.75 (m, 2H), 1.71 - 1.62 (m, 1H). | |
| | | Exchangeable —NH₂ protons were not observed. | |
| | | LCMS method G: m/z 433 (M+H)⁺ (ES+), at 0.52 min, UV active | |
| 2-29 | 5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one | Synthetic route q | 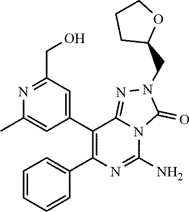 [SMILES: Cc1cc(-c2c(-c3ccccc3)nc(N)n3c(=O)n(C[C@H]4CCCO4)nc23)cc(CO)n1] |
| | | Intermediates 1 & 62 (step 1); intermediate 34 (step 2) | |
| | | (400 MHz, DMSO-d6) δ: 7.28 - 7.24 (m, 5H), 7.12 (s, 1H), 6.84 (s, 1H), 5.32-5.19 (m, 1H), 4.40 - 4.29 (m, 2H), 4.13 - 4.12 (m, 1H), 3.88 - 3.82 (m, 1H), 3.77 - 3.72 (m, 2H), 3.70 - 3.59 (m, 1H), 2.30 (s, 3H), 1.95 - 1.94 (m, 1H), 1.93 - 1.91 (m, 2H), 1.86 - 1.85 (m, 1H). | |
| | | Exchangeable —NH₂ protons were not observed. | |
| | | LCMS method A: m/z 433 (M+H)⁺ (ES+), at 2.25 min, UV active | |
| 2-30 | 5-amino-8-[2-(hydroxymethyl)-6-methyl-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one | Synthetic route p | 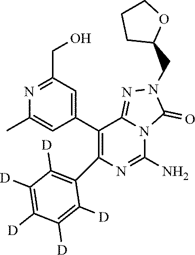 [SMILES: [2H]c1c([2H])c([2H])c(-c2nc(N)n3c(=O)n(C[C@H]4CCCO4)nc3c2-c2cc(C)nc(CO)c2)c([2H])c1[2H]] |
| | | Intermediates 38 & 62 (step 1) | |
| | | ¹H NMR (400 MHz, DMSO-d6) δ: 7.13 (s, 1H), 6.85 (s, 1H), 5.27 (s, 1H), 4.44 (s, 2H), 3.88-3.86 (m, 1H), 3.78-3.76 (m, 1H), 3.73 - 3.68 (m, 2H), 3.34-3.32 (m, 1H), 2.45 (s, 3H), 2.30-2.29 (m, 1H), 1.97-1.95 (m, 2H), 1.92-1.91 (m, 1H). | |
| | | Exchangeable —NH₂ Protons were not observed. | |
| | | LCMS Method C: m/z 438 (M+H)⁺ (ES+), at 1.29 min, UV active | |
| 2-31 | 5-amino-7-(4-fluorophenyl)-8-[2-(hydroxymethyl)-6-methyl-4-pyridyl]-2-[[(2R)-tetrahydrofuran-2-yl]methyl]-[1,2,4]triazolo[4,3-c]pyrimidin-3-one | Synthetic route p (step 2 purified by prep HPLC method A) | 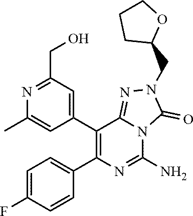 [SMILES: Cc1cc(-c2c(-c3ccc(F)cc3)nc(N)n3c(=O)n(C[C@H]4CCCO4)nc23)cc(CO)n1] |
| | | Intermediates 42 & 62 (step 1) | |
| | | ¹H NMR (400 MHz, DMSO-d6) δ: 7.32-7.29 (m, 2H), 7.13 - 7.07 (m, 3H), 6.89 (s, 1H), 5.27 (t, J = 5.7 Hz, 1H), 4.44 (d, J = 5.7 Hz, 2H), 4.13 (t, J = 6.0 Hz, 1H), 3.88-3.82 (m, 1H), 3.75-3.71 (m, 2H), 3.65-3.61 (m, 1H), 2.33 (s, 3H), 1.86-1.80 (m, 3H), 1.68-1.65 (m, 1H). | |
| | | Exchangeable —NH₂ Protons were not observed. | |
| | | LCMS Method A: m/z 451 (M+H)⁺ (ES+), at 1.88 min, UV active | |
| 2-32 | 5-amino-8-[2-chloro-6-(hydroxymethyl)-4-pyridyl]-7-phenyl-2-[[(2R)-tetrahydrofuran-2-yl]methyl]-[1,2,4]triazolo[4,3-c]pyrimidin-3-one | Synthetic route q | 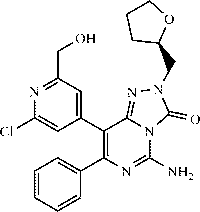 [SMILES: Nc1nc(-c2ccccc2)c(-c2cc(Cl)nc(CO)c2)c2nn(C[C@H]3CCCO3)c(=O)n12] |
| | | Intermediates 1 & 62 (step 1), 44 (step 2) | |
| | | ¹H NMR (400 MHz, DMSO-d6) δ: 8.52 (s, 2H), 7.32-7.29 (m, 6H), 7.05 (s, 1H), 5.45 (t, J = 5.6 Hz, 1H), 4.43 (d, J = 5.6 Hz, 2H), 4.16-4.13 (m, 1H), 3.89-3.84 (m, 1H), 3.78-3.73 (m, 2H), 3.65-3.62 (m, 1H), 1.97-1.80 (m, 3H), 1.69-1.67 (m, 1H). | |
| | | LCMS Method A: m/z 453 (M+H)⁺ (ES+), at 2.29 min, UV active | |
| 2-33 | 5-amino-8-[2-chloro-6-(hydroxymethyl)-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one | Synthetic route q | 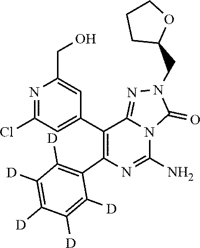 [SMILES: [2H]c1c([2H])c([2H])c(-c2nc(N)n3c(=O)n(C[C@H]4CCCO4)nc3c2-c2cc(Cl)nc(CO)c2)c([2H])c1[2H]] |
| | | Intermediates 39 & 62 (step 1), 44 (step 2) | |
| | | ¹H NMR (400 MHz, DMSO-d6) δ: 8.40-7.80 (m, 2H), 7.31 (s, 1H), 7.05 (s, 1H), 5.46 (t, J = 4.0 Hz, 1H), 4.43 (d, J = 4.0 Hz, 2H), 4.15-4.12 (m, 1H), 3.89-3.83 (m, 1H), 3.76-3.72 (m, 2H), 3.65-3.61 (m, 1H), 1.99-1.86 (m, 1H), 1.85-1.08 (m, 2H), 1.70-7.67 (m, 1H). | |
| | | LCMS Method C: m/z 458 (M+H)⁺ (ES+), at 1.73 min, UV active | |
| 2-34 | 5-amino-8-[2-chloro-6-(hydroxymethyl)-4-pyridyl]-7-(4-fluorophenyl)-2-[[(2R)-tetrahydrofuran-2-yl]methyl]-[1,2,4]triazolo[4,3-c]pyrimidin-3-one | Synthetic route q | 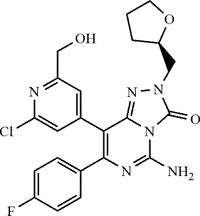 [SMILES: Nc1nc(-c2ccc(F)cc2)c(-c2cc(Cl)nc(CO)c2)c2nn(C[C@H]3CCCO3)c(=O)n12] |
| | | Intermediates 26 & 62 (step 1), 44 (step 2) | |
| | | ¹H NMR (400 MHz, DMSO-d6) δ 8.21 (s, 2H), 7.34-7.31 (m, 3H), 7.28-7.12 (m, 3H), 5.46 (t, J = 5.2 Hz, 1H), 4.44 (d, J = 5.2 Hz, 2H), 4.16-4.13 (m, 1H), 3.85 (d, J = 7.2 Hz, 1H), 3.77-3.73 (m, 2H), 3.62 (d, J = 7.6 Hz, 1H), 1.97-1.94 (m, 1H), 1.87-1.80 (m, 2H), 1.69-1.66 (m, 1H). | |
| | | LCMS Method C: m/z 471 (M+H)⁺ (ES+), at 1.81 min, UV active | |
| 2-35 | 5-amino-8-[2-(hydroxymethyl)-6-methoxy-4-pyridyl]-7-phenyl-2-[[(2R)-tetrahydrofuran-2-yl]methyl]-[1,2,4]triazolo[4,3-c]pyrimidin-3-one | Synthetic route q | 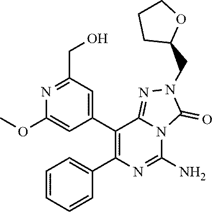 [SMILES: COc1cc(-c2c(-c3ccccc3)nc(N)n3c(=O)n(C[C@H]4CCCO4)nc23)cc(CO)n1] |
| | | Intermediates 1 & 62 (step 1), 46 (step 2) | |
| | | ¹H NMR (400 MHz, DMSO-d6) δ 7.34 - 7.22 (m, 5H), 6.87 (s, 1H), 6.45 (s, 1H), 5.24 (t, J = 5.8 Hz, 1H), 4.38 (d, J = 5.8 Hz, 2H), 4.19 ― 4.05 (m, 1H), 3.91 ― 3.80 (m, 1H), 3.74 (s, 3H), 3.66 - 3.53 (m, 1H), 2.73 - 2.63 (m, 2H), 2.38 - 2.29 (m, 2H), 2.03 - 1.55 (m, 2H). | |
| | | Exchangeable —NH₂ Protons were not observed. | |
| | | LCMS Method A: m/z 449 (M+H)⁺ (ES+), at 2.07 min, UV active | |
| 2-36 | 5-amino-8-[2-(hydroxymethyl)-6-methoxy-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one | Synthetic route q | 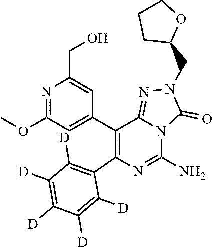 [SMILES: [2H]c1c([2H])c([2H])c(-c2nc(N)n3c(=O)n(C[C@H]4CCCO4)nc3c2-c2cc(CO)nc(OC)c2)c([2H])c1[2H]] |
| | | Intermediates 39 & 62 (step 1), 46 (step 2) | |
| | | ¹H NMR (400 MHz, DMSO-d6) δ: 6.87 (s, 1H), 6.46 (s, 1H), 5.25 (t, J = 5.6 Hz, 1H), 4.38 (d, J = 5.6 Hz, 2H), 4.15-4.12 (m, 1H), 3.88-3.85 (m, 1H), 3.76-3.72 (m, 4H), 3.64 (d, J = 7.2 Hz, 1H), 1.92-1.91 (m, 4H), 1.88-1.86 (m, 1H). | |
| | | Exchangeable —NH₂ Protons were not observed. | |
| | | LCMS Method C: m/z 454 (M+H)⁺ (ES+), at 1.68 min, UV active | |
| 2-37 | 5-amino-7-(4-fluorophenyl)-8-[2-(hydroxymethyl)-6-methoxy-4-pyridyl]-2-[[(2R)-tetrahydrofuran-2-yl]methyl]-[1,2,4]triazolo[4,3-c]pyrimidin-3-one | Synthetic route q | 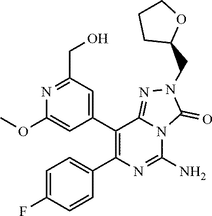 [SMILES: COc1cc(-c2c(-c3ccc(F)cc3)nc(N)n3c(=O)n(C[C@H]4CCCO4)nc23)cc(CO)n1] |
| | | Intermediates 26 & 62 (step 1), 46 (step 2) | |
| | | ¹H NMR (400 MHz, DMSO-d6) δ: 7.35-7.33 (m, 2H), 7.14-7.09 (m, 2H), 6.84 (s, 1H), 6.51 (s, 1H), 5.25 (t, J = 5.6 Hz, 1H), 4.38 (d, J = 5.6 Hz, 2H), 4.38-4.15 (m, 1H), 4.12-4.10 (m, 1H), 3.88-3.84 (m, 5H), 3.78-3.78 (m, 1H), 1.99 - 1.95 (m, 3H), 1.90-1.87 (m, 1H). | |
| | | Exchangeable —NH₂ Protons were not observed. | |
| | | LCMS Method C: m/z 467 (M+H)⁺ (ES+), at 1.76 min, UV active | |
| 2-38 | 5-amino-8-[2-(hydroxymethyl)-6-(trifluoromethyl)-4-pyridyl]-7-phenyl-2-[[(2R)-tetrahydrofuran-2-yl]methyl]-[1,2,4]triazolo[4,3-c]pyrimidin-3-one | Synthetic route q | 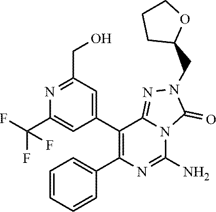 [SMILES: Nc1nc(-c2ccccc2)c(-c2cc(CO)nc(C(F)(F)F)c2)c2nn(C[C@H]3CCCO3)c(=O)n12] |
| | | Intermediates 1 & 62 (step 1), 48 (step 2) | |
| | | ¹H NMR (400 MHz, DMSO-d6) δ: 7.72 (s, 1H), 7.34-7.29 (m, 6H), 5.55 (t, J = 5.8 Hz, 1H), 4.55 (d, J = 5.8 Hz, 2H), 4.16-4.13 (m, 1H), 3.90-3.86 (m, 1H), 3.75 (t, J = 4.2 Hz, 2H), 3.66-3.64 (m, 1H), 2.01-1.99 (m, 1H), 1.97-1.96 (m, 2H), 1.90-1.89 (m, 1H). | |
| | | Exchangeable —NH₂ Protons were not observed. | |
| | | LCMS Method C: m/z 487 (M+H)⁺ (ES+), at 1.94 min, UV active | |
| 3-1 | 5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one | Synthetic route ad | 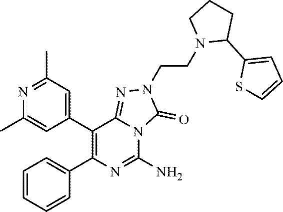 [SMILES: Cc1cc(-c2c(-c3ccccc3)nc(N)n3c(=O)n(CCN4CCCC4c4cccs4)nc23)cc(C)n1] |
| | | ¹H NMR (400 MHz, DMSO-d6) δ 8.24 (s, 1H), 7.63 (s, 1H), 7.32 ― 7.15 (m, 5H), 7.10 (d, J = 4.9 Hz, 1H), 6.88 ― 6.76 (m, 2H), 6.60 (s, 2H), 3.96 (d, J = 14.2 Hz, 1H), 3.84 - 3.64 (m, 2H), 3.51 ― 3.40 (m, 1H), 3.08 (d, J = 15.6 Hz, 1H), 2.54 (s, 2H), 2.28 (s, 8H), 2.04―1.62 (m, 2H). | |
| | | LCMS Method F: m/z 512 (M+H)⁺ (ES+), at 0.75 min, UV active | |
| 3-2 | 5-amino-8-(2,6-dimethyl-4-pyridyl)-7-phenyl-2-[2-[[1-(pyridine-3-carbonyl)pyrrolidin-3-yl]amino]ethyl]-[1,2,4]triazolo[4,3-c]pyrimidin-3-one | Synthetic route ad | 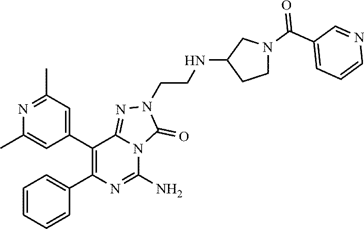 [SMILES: Cc1cc(-c2c(-c3ccccc3)nc(N)n3c(=O)n(CCNC4CCN(C(=O)c5cccnc5)C4)nc23)cc(C)n1] |
| | | Intermediates 71 & 73 | |
| | | ¹H NMR (400 MHz, Chloroform-d) δ: 8.81 ― 8.74 (m, 1H), 8.70 - 8.61 (m, 1H), 7.90 - 7.79 (m, 1H), 7.38 - 7.27 (m, 2H), 7.26 - 7.17 (m, 3H), 6.81 (d, J = 11.6 Hz, 2H), 4.08 - 3.91 (m, 2H), 3.88 - 3.33 (m, 5H), 3.28 - 2.87 (m, 3H), 2.47 - 2.34 (m, 7H), 2.24 ― 1.96 (m, 1H), 1.86 - 1.69 (m, 1H), 1.44 ― 1.23 (m, 1H). | |
| | | One exchangeable NH not observed. | |
| | | LCMS Method F: m/z 548 (M-H)⁻ (ES-), at 0.66 min, UV active | |
| 3-3 | 5-amino-8-(2,6-dimethyl-4-pyridyl)-2-[2-[methyl(1H-pyrazol-4-yl)amino]ethyl]-7-phenyl-[1,2,4]triazolo[4,3-c]pyrimidin-3-one | Synthetic route ad | 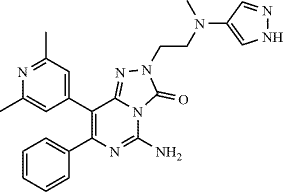 [SMILES: Cc1cc(-c2c(-c3ccccc3)nc(N)n3c(=O)n(CCN(C)c4cn[nH]c4)nc23)cc(C)n1] |
| | | Intermediates 71 & 74 | |
| | | ¹H NMR (400 MHz, Chloroform-d) δ: 7.26 (s, 6H), 7.06 - 7.02 (m, 2H), 6.85 - 6.82 (m, 2H), 4.04 (t, J = 5.9 Hz, 2H), 3.49 - 3.42 (m, 3H), 2.79 (s, 3H), 2.44 (s, 6H). | |
| | | One exchangeable NH not observed. | |
| | | LCMS Method F: m/z 456 (M+H)⁺ (ES+), at 0.78 min, UV active | |
| 3-4 | 5-amino-2-[[1-[[2-(aminomethyl)phenyl]methyl]pyrrolidin-2-yl]methyl]-8-(2,6-dimethyl-4-pyridyl)-7-phenyl-[1,2,4]triazolo[4,3-c]pyrimidin-3-one | Synthetic route z | 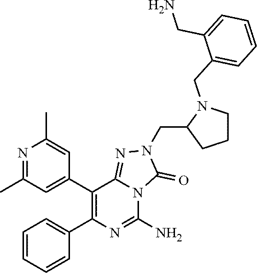 [SMILES: Cc1cc(-c2c(-c3ccccc3)nc(N)n3c(=O)n(CC4CCCN4Cc4ccccc4CN)nc23)cc(C)n1] |
| | | Intermediates 75 & 6 | |
| | | ¹H NMR (400 MHz, Chloroform-d) δ: 7.32 ― 7.19 (m, 10H), 7.19 ― 7.05 (m, 1H), 6.80 (s, 2H), 4.08 (d, J = 12.6 Hz, 1H), 3.99 - 3.86 (m, 2H), 3.84 - 3.71 (m, 2H), 3.52 - 3.39 (m, 1H), 3.12 (s, 1H), 2.95 - 2.81 (m, 1H), 2.40 (s, 7H), 2.04 ― 1.63 (m, 6H). | |
| | | LCMS Method F: m/z 535 (M+H)⁺ (ES+), at 0.83 min, UV active | |
| 4-1 | 5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one | Synthetic route ae | 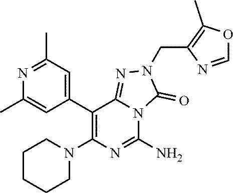 [SMILES: Cc1cc(-c2c(N3CCCCC3)nc(N)n3c(=O)n(Cc4ncoc4C)nc23)cc(C)n1] |
| | | ¹H NMR (400 MHz, DMSO-d6) δ: 8.14 (s, 1H), 7.12 (s, 2H), 4.74 (s, 2H), 3.13 (s, 4H), 2.38 - 2.33 (m, 9H), 1.49 (s, 2H), 1.41 (s, 4H). | |
| | | Exchangeable NH₂— Protons were not observed. | |
| | | LCMS Method C: m/z 435 (M+H)⁺ (ES+), at 1.38 min, UV active | |

### Example 5: Adenosine Receptor Binding Assay

Inhibition binding assays were performed using 0.2 µg of membranes prepared from HEK293 cells infected with BacMam human adenosine A_{2A} receptor or 1.4 µg of membranes prepared from HEK293 cells infected with BacMam human adenosine A1 receptor. Membranes were incubated in 50 mM Tris-HCl (HEK293-hA_{2A}; pH 7.4) or 50 mM Tris-HCl, 100 mM NaCl, 10 mM MgCl2 (CHO-hA₁; pH 7.4) in the presence of varying concentrations of test compound and 1 nM [³H]ZM241385 (HEK293-hA_{2A}) or [³H]DPCPX (CHO-hA₁) at 25° C. for 1 h. The assay was then terminated by rapid filtration onto GF/B grade Unifilter plates using a TomTec cell harvester, followed by 5 × 0.5 ml washes with ddH2O. Nonspecific binding was defined in the presence of 1 µM CGS 15943 (HEK293-hA_{2A}) or 1 µM DPCPX (CHO-hA₁). Bound radioactivity was determined by liquid scintillation counting and inhibition curves were analysed using a four-parameter logistic equation. IC₅₀ values were converted to Ki values with the Cheng-Prusoff equation using a KD value derived from saturation binding studies. Results are summarized in Table 3.

TABLE 3

Adenosine receptor binding

| Example | A₁ pKi | A_{2A} pKi |
|---|---|---|
| 1-1 | 6.2 | 7.5 |
| 1-2 | 6.1 | 8.2 |
| 1-3 | 5.9 | 7.9 |
| 1-4 | <5.4 | 6.4 |
| 1-5 | <5.4 | 7.2 |
| 1-6 | 5.7 | 7.2 |
| 1-7 | 6.6 | 8.0 |
| 1-8 | 6.7 | 7.8 |
| 1-9 | 7.0 | 9.1 |
| 1-10 | 6.8 | 9.2 |
| 1-11 | 7.8 | 9.8 |
| 1-12 | 7.1 | 9.0 |
| 1-13 | 6.8 | 8.9 |
| 1-14 | 6.6 | 9.2 |
| 1-15 | 6.7 | 8.9 |
| 1-16 | 7.2 | 9.4 |
| 1-17 | 6.2 | 8.2 |
| 1-18 | 6.9 | 8.5 |
| 1-19 | 7.2 | 9.6 |
| 1-20 | 6.6 | 8.7 |
| 1-21 | 6.4 | 8.3 |
| 1-22 | 7.4 | 10.1 |
| 1-23 | 6.6 | 8.3 |
| 1-24 | 7.6 | 9.2 |
| 1-25 | 7.1 | 8.6 |
| 1-26 | <5.4 | 6.4 |
| 1-27 | 6.6 | 8.1 |
| 1-28 | 6.5 | 7.7 |
| 1-29 | 7.0 | 8.6 |
| 1-30 | 6.6 | 8.9 |
| 1-31 | 7.0 | 9.6 |
| 1-32 | 7.0 | 9.5 |
| 1-33 | 7.1 | 8.1 |
| 1-34 | 7.5 | 9.2 |
| 1-35 | 7.4 | 8.4 |
| 1-36 | 7.3 | 8.0 |
| 1-37 | 6.8 | 8.8 |
| 1-38 | 7.8 | 9.5 |
| 1-39 | 6.8 | 9.5 |
| 1-40 | 6.9 | 8.8 |
| 1-41 | 7.2 | 9.1 |
| 1-42 | 6.7 | 8.5 |
| 1-43 | 6.7 | 9.1 |
| 1-44 | 6.8 | 8.4 |
| 1-45 | 7.2 | 9.3 |
| 1-46 | 6.4 | 9.3 |
| 1-47 | 6.8 | 9.5 |
| 1-48 | 6.8 | 8.9 |
| 1-49 | 6.5 | 8.8 |
| 1-50 | 6.5 | 9.5 |
| 1-51 | 6.4 | 9.4 |
| 1-52 | 6.7 | 8.6 |
| 1-53 | 6.5 | 9.2 |
| 1-54 | 7.2 | 9.7 |
| 1-55 | 7.5 | 9.4 |
| 1-56 | 8.3 | 9.8 |
| 1-57 | 6.9 | 10.0 |
| 1-58 | 7.1 | 9.5 |
| 1-59 | 6.8 | 9.9 |
| 1-60 | 6.8 | 9.9 |
| 1-61 | 6.8 | 9.5 |
| 1-62 | 6.9 | 9.6 |
| 1-63 | 6.5 | 10.1 |
| 1-64 | 6.5 | 9.6 |
| 1-65 | 7.1 | 10.0 |
| 1-66 | 7.5 | 9.7 |
| 1-67 | 8.0 | 10.0 |
| 2-1 | 6.8 | 7.7 |
| 2-2 | 5.7 | 7.4 |
| 2-3 | 6.1 | 7.8 |
| 2-4 | 6.6 | 7.9 |
| 2-5 | 6.1 | 7.4 |
| 2-6 | 6.2 | 9.1 |
| 2-7 | 7.4 | 9.3 |
| 2-8 | 6.8 | 9.0 |
| 2-9 | 6.1 | 8.4 |
| 2-10 | 6.3 | 8.2 |
| 2-11 | 6.0 | 8.7 |
| 2-12 | 6.4 | 8.1 |
| 2-13 | 6.3 | 8.2 |
| 2-14 | 7.0 | 8.5 |
| 2-15 | 5.7 | 7.5 |
| 2-16 | 6.7 | 9.2 |
| 2-17 | | 8.6 |
| 2-18 | 6.4 | 8.2 |
| 2-19 | 6.2 | 8.0 |
| 2-20 | 6.3 | 8.5 |
| 2-21 | 6.4 | 8.4 |
| 2-22 | 5.9 | 8.2 |
| 2-23 | 5.9 | 8.2 |
| 2-24 | 6.5 | 8.8 |
| 2-25 | 6.1 | 8.8 |
| 2-26 | <5.7 | 6.9 |
| 2-27 | 6.7 | 9.6 |
| 2-28 | 6.5 | 9.1 |
| 2-29 | 6.4 | 9.3 |
| 2-30 | 6.3 | 9.0 |
| 2-31 | 6.1 | 8.4 |
| 2-32 | 6.6 | 9.6 |
| 2-33 | 6.8 | 9.5 |
| 2-34 | 6.3 | 9.4 |
| 2-35 | 5.9 | 9.2 |
| 2-36 | 5.9 | 9.1 |
| 2-37 | 5.5 | 9.0 |
| 2-38 | 6.3 | 8.9 |
| 3-1 | 7.6 | 8.9 |
| 3-2 | 5.6 | 6.9 |
| 3-3 | 5.7 | 7.2 |
| 3-4 | 6.3 | 8.4 |
| 4-1 | <6.0 | 8.9 |

### Example 6: CB-1 Receptor Binding and Antagonism

Receptor binding: Evaluation of the affinity of compounds for the agonist site of the human CB-1 cannabinoid receptor in transfected CHO cells determined in a radioligand binding assay: Cell membrane homogenates (20 µg protein) are incubated for 120 min at 37° C. with 0.5 nM [³H]CP 55940 in the absence or presence of the test compound in a buffer containing 50 mM Tris-HCl (pH 7.4), 5 mM MgCl2, 2.5 mM EDTA and 0.3% BSA. Nonspecific binding is determined in the presence of 10 µM WIN 55212-2.

Following incubation, the samples are filtered rapidly under vacuum through glass fiber filters (GF/B, Packard) presoaked with 0.3% PEI and rinsed several times with an ice-cold buffer containing 50 mM Tris-HCl (pH 7.4) and 0.5% BSA using a 96-sample cell harvester (Unifilter, Packard). The filters are dried then counted for radioactivity in a scintillation counter (Topcount, Packard) using a scintillation cocktail (Microscint 0, Packard).

The standard reference compound is CP 55940 which is tested in each experiment at several concentrations to obtain a competition curve from which its IC₅₀ is calculated.

Receptor antagonism: Evaluation of the antagonist activity of compounds at the human CB1 receptor expressed in transfected CHO cells, determined by measuring their effects on agonist-induced cAMP modulation using the HTRF detection method.

\The cells are suspended in HBSS buffer (Invitrogen) complemented with 20 mM HEPES (pH 7.4), then distributed in microplates at a density of 5.10³ cells/well and preincubated for 5 min at room temperature in the presence of either of the following: HBSS (stimulated control), the reference antagonist AM 281 at 3 µM (basal control) or various concentrations (IC₅₀ determination), or the test compounds.

Thereafter, the reference agonist CP 55940 and the adenylyl cyclase activator NKH 477 are added at respective final concentrations of 3 nM and 3 µM.

For basal control measurements, CP 55940 is omitted from the wells containing 3 µM AM 281.

Following 20 min incubation at 37° C., the cells are lysed and the fluorescence acceptor (D2-labeled cAMP) and fluorescence donor (anti-cAMP antibody labeled with europium cryptate) are added.

After 60 min at room temperature, the fluorescence transfer is measured at λₑₓ=337 nm and λₑₘ=620 and 665 nm using a microplate reader (Rubystar, BMG). The cAMP concentration is determined by dividing the signal measured at 665 nm by that measured at 620 nm (ratio).

The results are expressed as a percent inhibition of the control response to 3 nM CP 55940.

The standard reference antagonist is AM 281, which is tested in each experiment at several concentrations to generate a concentration-response curve from which its IC₅₀ value is calculated.

In Table 4, blank entries for Ki indicate that the observed binding was too weak to measure a Ki value.

TABLE 4

CB1 receptor binding and functional assay

| Example | Binding Kᵢ (µM) | Antag IC₅₀ (µM) |
|---|---|---|
| 1-5 | | > 100 |
| 1-14 | 50.5 | 29.2 |
| 1-16 | | > 100 |
| 1-22 | 29.6 | > 100 |
| 1-31 | 26.1 | 12.1 |
| 1-35 | | > 100 |
| 1-36 | | > 100 |
| 1-37 | | > 100 |
| 1-38 | | > 100 |
| 1-43 | 86.9 | > 100 |
| 1-46 | | > 100 |
| 1-50 | 86.1 | > 100 |
| 1-51 | 80.3 | > 100 |
| 1-52 | | > 100 |
| 1-53 | | > 100 |
| 1-55 | | > 100 |
| 1-58 | | > 100 |
| 1-60 | 66.7 | > 100 |
| 1-61 | | > 100 |
| 1-64 | 40.8 | > 100 |
| 1-66 | | > 100 |
| 1-67 | | > 100 |
| 2-14 | 36.4 | 35.8 |
| 2-15 | | > 100 |
| 2-28 | | > 100 |
| 2-29 | | > 100 |
| 2-32 | | > 100 |
| 2-35 | | > 100 |
| 2-38 | | > 100 |
| 3-1 | 29.2 | 10.1 |
| 3-4 | 76.4 | > 100 |

Other embodiments are within the scope of the following claims.

What is claimed is:

1. A compound of Formula (I): 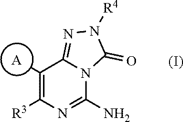
or a pharmaceutically acceptable salt thereof, wherein: ring A is: 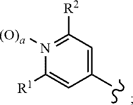
each R¹ and each R², independently, is halo, C₁₋₃alkyl, -O-C₁₋₃alkyl, —CO₂R^{a}, or —NR⁷R⁸; wherein alkyl is optionally substituted with one or more substituents independently selected from —OR^{a} and halo;
R³ is C₁₋₆alkyl, C₂₋₆alkenyl, C₂₋₆alkynyl, aryl, heterocyclyl, heteroaryl, halo, —OR^{a}, —NR^{a}R^{b}, —CO₂R^{a}, —CONR^{a}R^{b}, —NR^{a}C(O)—R^{a}, or —NHC(O)—OR^{a};
wherein heterocyclyl and heteroaryl, independently, include from 1 to 4 heteroatoms independently selected from N, O, and S(O)ₖ;
wherein R³ is optionally substituted with from one to three substituents selected from halo, cyano, —R^{a}, and —OR^{a};
R⁴ is —(CHR^{c})ᵢ₋(NR^{a})ⱼ—R⁵;
R⁵ is a 5-membered heterocyclyl or 5-membered heteroaryl, each including from 1 to 4 heteroatoms independently selected from N, O, and S(O)ₖ;
wherein one or two ring atoms of R⁵ is optionally replaced by —C(═O)—;
wherein R⁵ is optionally substituted with from one to four groups —X—R⁶; each X, independently, is a bond, —O—, —NR^{a}—, —S(O)ₖ—, —(CH₂)ₘ—, or —C(O)—;
each R⁶, independently, is H, halo, —OR^{a}, C₁₋₆alkyl, C₃₋₈cycloalkyl, heterocyclyl, heteroaryl, aryl, —CO₂R^{a}, —C(O)NR^{a}R^{b}, —(CH₂)ₙ—NR^{a}R^{b}, or cyano;
wherein each of heterocyclyl and heteroaryl includes from 1 to 4 heteroatoms independently selected from N, O, and S(O)ₖ;
wherein one or two ring atoms of each C₃₋₈cycloalkyl, heterocyclyl, heteroaryl, or aryl, independently is optionally replaced by —C(═O)—;
wherein each of alkyl, cycloalkyl, heterocyclyl, heteroaryl, and aryl is optionally substituted with one or more substituents independently selected from —R^{a}, —OR^{a}, —(CH₂)ₙ—NR^{a}R^{b}, and halo;
each R⁷ and each R⁸, independently, is R^{a};
or R⁷ and R⁸ together with the atom to which they are attached form a 3- to 8-membered heterocyclyl optionally substituted with one or more substituents independently selected from
—OR^{a} and halo;
each R^{a} and each R^{b}, independently, is H, C₁₋₆alkyl, C₃₋₈cycloalkyl, or C₄₋₉cycloalkylalkyl;
wherein each R^{a} and each R^{b}, independently, is optionally substituted with one or more substituents independently selected from —OH and halo;
each R^{c}, independently, is H, halo, C₁₋₃alkyl, or —(CH₂)ₙ—NR^{a}R^{b}; wherein alkyl is optionally substituted with one or more substituents independently selected from —OR^{a} and halo;
a is 0 or 1;
i is 0, 1, 2, or 3;
j is 0 or 1;
each k, independently, is 0, 1, or 2;
each m, independently, is 1 or 2; and
each n, independently, is 0 or 1.

2. The compound of claim 1, wherein the compound is a selective adenosine receptor antagonist with respect to CB-1.

3. The compound of claim 2, wherein the compound has a Ki for at least one of A2aR and A2bR of 100 nM or less, and has a Ki for CB-1 of 10,000 nM or more.

4. The compound of any one of claims 1 to 3, wherein i is 1 and R^{c} is H or C₁₋₃ alkyl.

5. The compound of any one of claims 1 to 4, wherein R⁵ is selected from imidazolyl, 1,2,4-oxadiazolyl, 1,2,5-oxadiazolyl, 1,3,4-oxadiazolyl, 1,2-oxazolyl, 1,3-oxazolyl, pyrazolyl, pyrrolidinyl, pyrrolyl, tetrahydrofuranyl, tetrazolyl, thiophenyl, 1,2,3-triazolyl, and 1,3,4-triazolyl, wherein R⁵ is optionally substituted with from one to four groups -X-R⁶.

6. The compound of claim 5, wherein X is a bond and R⁶ is C₁₋₆alkyl.

7. The compound of any one of claims 1 to 6, wherein R³ is phenyl optionally substituted with fluoro or chloro.

8. The compound of any one of claims 1 to 7, wherein R¹ and R² are each independently selected from halo, —CH₃, —CH₂OH, or —OCH₃.

9. A compound of Formula (II): 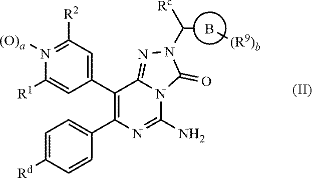
or a pharmaceutically acceptable salt thereof, wherein:
each R¹ and each R², independently, is halo, C₁₋₃alkyl, or -O-C₁₋₃alkyl; wherein alkyl is optionally substituted with one or more substituents independently selected from —OH and halo;
ring B is a 5-membered heterocyclyl or 5-membered heteroaryl, each including from 1 to 4 heteroatoms independently selected from N and O;
each R⁹, independently, is halo or C₁₋₃alkyl; wherein alkyl is optionally substituted with one or more substituents independently selected from —OH and halo;
each R^{a} and each R^{b}, independently, is H, C₁₋₆alkyl, C₃₋₈cycloalkyl, or C₄₋₉cycloalkylalkyl; wherein each R^{a} and each R^{b}, independently, is optionally substituted with one or more substituents independently selected from —OH and halo;
R^{c} is H, halo, C₁₋₃alkyl, or —(CH₂)ₙ—NR^{a}R^{b}; wherein alkyl is optionally substituted with one or more substituents independently selected from —OR^{a} and halo;
R^{d} is H or halo;
a is 0 or 1;
b is 0, 1, or 2; and
n is 0 or 1.

10. The compound of claim 9, wherein ring B is tetrahydrofuranyl or 1,3-oxazolyl, b is 0 or 1, and each R⁹, independently, is C₁₋₃alkyl.

11. A compound, or a pharmaceutically acceptable salt thereof, selected from the group consisting of:
5-amino-8-(2,6-dimethyl-4-pyridyl)-7-phenyl-2-(2-pyrazol-1-ylethyl)-[1,2,4]triazolo[4,3-c]pyrimidin-3-one;
5-amino-8-(2,6-dimethyl-4-pyridyl)-2-[2-(1-methylimidazol-2-yl)ethyl]-7-phenyl-[1,2,4]triazolo[4,3-c]pyrimidin-3-one;
5-amino-8-(2,6-dimethyl-4-pyridyl)-2-[2-(1H-imidazol-2-yl)ethyl]-7-phenyl-[1,2,4]triazolo[4,3-c]pyrimidin-3-one;
5-amino-8-(2,6-dimethyl-4-pyridyl)-7-phenyl-2-[2-(1H-tetrazol-5-yl)ethyl]-[1,2,4]triazolo[4,3-c]pyrimidin-3-one;
5-amino-8-(2,6-dimethyl-4-pyridyl)-2-[2-(1-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-methyltetrazol-5-yl)ethyl]-7-phenyl-[1,2,4]triazolo[4,3-c]pyrimidin-3-one;
5-amino-8-(2,6-dimethyl-4-pyridyl)-2-[2-(2-ethylpyrazol-3-yl)ethyl]-7-phenyl-[1,2,4]triazolo[4,3-c]pyrimidin-3-one;
5-amino-8-(2,6-dimethyl-4-pyridyl)-7-phenyl-2-[1-(2-thienyl)ethyl]-[1,2,4]triazolo[4,3-c]pyrimidin-3-one;
5-amino-8-(2,6-dimethyl-4-pyridyl)-2-(oxazol-2-ylmethyl)-7-phenyl-[1,2,4]triazolo[4,3-c]pyrimidin-3-one;
5-amino-8-(2,6-dimethyl-4-pyridyl)-2-(oxazol-4-ylmethyl)-7-phenyl-[1,2,4]triazolo[4,3-c]pyrimidin-3-one;
5-amino-8-(2,6-dimethyl-4-pyridyl)-2-[(1-methylimidazol-2-yl)methyl]-7-phenyl-[1,2,4]triazolo[4,3-c]pyrimidin-3-one;
5-amino-8-(2,6-dimethyl-4-pyridyl)-2-[(2-methylpyrazol-3-yl)methyl]-7-phenyl-[1,2,4]triazolo[4,3-c]pyrimidin-3-one;
5-amino-8-(2,6-dimethyl-4-pyridyl)-2-(isoxazol-3-ylmethyl)-7-phenyl-[1,2,4]triazolo[4,3-c]pyrimidin-3-one;
5-amino-8-(2,6-dimethyl-4-pyridyl)-2-[(5-methylisoxazol-3-yl)methyl]-7-phenyl-[1,2,4]triazolo[4,3-c]pyrimidin-3-one;
5-amino-8-(2,6-dimethyl-4-pyridyl)-2-[(2-methyloxazol-4-yl)methyl]-7-phenyl-[1,2,4]triazolo[4,3-c]pyrimidin-3-one;
5-amino-8-(2,6-dimethyl-4-pyridyl)-2-[(1-methylpyrazol-3-yl)methyl]-7-phenyl-[1,2,4]triazolo[4,3-c]pyrimidin-3-one;
5-amino-8-(2,6-dimethyl-4-pyridyl)-2-[(5-methyl-1,3,4-oxadiazol-2-yl)methyl]-7-phenyl-[1,2,4]triazolo[4,3-c]pyrimidin-3-one;
5-amino-8-(2,6-dimethyl-4-pyridyl)-2-[(3-methylimidazol-4-yl)methyl]-7-phenyl-[1,2,4]triazolo[4,3-c]pyrimidin-3-one;
5-amino-8-(2,6-dimethyl-4-pyridyl)-2-[(4-methyl-1,2,5-oxadiazol-3-yl)methyl]-7-phenyl-[1,2,4]triazolo[4,3-c]pyrimidin-3-one;
5-amino-8-(2,6-dimethyl-4-pyridyl)-2-[(5-methyl-1,2,4-oxadiazol-3-yl)methyl]-7-phenyl-[1,2,4]triazolo[4,3-c]pyrimidin-3-one;
5-amino-8-(2,6-dimethyl-4-pyridyl)-2-[(3-methyl-1,2,4-oxadiazol-5-yl)methyl]-7-phenyl-[1,2,4]triazolo[4,3-c]pyrimidin-3-one;
5-amino-8-(2,6-dimethyl-4-pyridyl)-2-[(5-methyloxazol-4-yl)methyl]-7-phenyl-[1,2,4]triazolo[4,3-c]pyrimidin-3-one;
5-amino-8-(2,6-dimethyl-4-pyridyl)-2-(1H-imidazol-5-ylmethyl)-7-phenyl-[1,2,4]triazolo[4,3-c]pyrimidin-3-one;
5-amino-8-(2,6-dimethyl-4-pyridyl)-2-(1H-imidazol-2-ylmethyl)-7-phenyl-[1,2,4]triazolo[4,3-c]pyrimidin-3-one;
5-amino-8-(2,6-dimethyl-4-pyridyl)-7-phenyl-2-(1H-pyrazol-5-ylmethyl)-[1,2,4]triazolo[4,3-c]pyrimidin-3-one;
5-amino-8-(2,6-dimethyl-4-pyridyl)-7-phenyl-2-(2H-tetrazol-5-ylmethyl)-[1,2,4]triazolo[4,3-c]pyrimidin-3-one;
5-amino-8-(2,6-dimethyl-4-pyridyl)-2-(1,3,4-oxadiazol-2-ylmethyl)-7-phenyl-[1,2,4]triazolo[4,3-c]pyrimidin-3-one;
5-amino-8-(2,6-dimethyl-4-pyridyl)-2-[(4-methyl-1,2,4-triazol-3-yl)methyl]-7-phenyl-[1,2,4]triazolo[4,3-c]pyrimidin-3-one;
5-amino-8-(2,6-dimethyl-4-pyridyl)-2-[(5-methyl-1H-triazol-4-yl)methyl]-7-phenyl-[1,2,4]triazolo[4,3-c]pyrimidin-3-one;
5-amino-8-(2,6-dimethyl-4-pyridyl)-2-[(2-methyltriazol-4-yl)methyl]-7-phenyl-[1,2,4]triazolo[4,3-c]pyrimidin-3-one;
5-amino-8-(2,6-dimethyl-4-pyridyl)-7-(4-fluorophenyl)-2-[(5-methyloxazol-4-yl)methyl]-[1,2,4]triazolo[4,3-c]pyrimidin-3-one;
5-amino-2-[(2,5-dimethyloxazol-4-yl)methyl]-8-(2,6-dimethyl-4-pyridyl)-7-(4-fluorophenyl)-[1,2,4]triazolo[4,3-c]pyrimidin-3-one;
5-amino-2-[[1-benzyl-3-(3-methoxyphenyl)pyrazol-4-yl]methyl]-8-(2,6-dimethyl-4-pyridyl)-7-phenyl-[1,2,4]triazolo[4,3-c]pyrimidin-3-one;
5-amino-8-(2,6-dimethyl-1-oxido-pyridin-1-ium-4-yl)-2-[(5-methyloxazol-4-yl)methyl]-7-phenyl-[1,2,4]triazolo[4,3-c]pyrimidin-3-one;
5-amino-2-[(2,5-dimethyloxazol-4-yl)methyl]-8-(2,6-dimethyl-1-oxido-pyridin-1-ium-4-yl)-7-(4-fluorophenyl)-[1,2,4]triazolo[4,3-c]pyrimidin-3-one;
5-amino-8-(2,6-dimethyl-1-oxido-pyridin-1-ium-4-yl)-2-[(1-methylimidazol-2-yl)methyl]-7-phenyl-[1,2,4]triazolo[4,3-c]pyrimidin-3-one;
5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one;
5-amino-8-(2-methoxy-6-methyl-4-pyridyl)-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-2-ylmethyl)-7-phenyl-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methyl-4-pyridyl]-2-(oxazol-4-ylmethyl)-7-phenyl-[1,2,4]triazolo[4,3-c]pyrimidin-3-one;
5-amino-8-[2-(hydroxymethyl)-6-methyl-4-pyridyl]-2-[(5-methylisoxazol-3-yl)methyl]-7-phenyl-[1,2,4]triazolo[4,3-c]pyrimidin-3-one;
5-amino-2-[(3,5-dimethylimidazol-4-yl)methyl]-8-[2-(hydroxymethyl)-6-methyl-4-pyridyl]-7-phenyl-[1,2,4]triazolo[4,3-c]pyrimidin-3-one;
5-amino-8-[2-(hydroxymethyl)-6-methyl-4-pyridyl]-2-[(1-methylpyrazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(4-methyl-1,2,5-oxadiazol-3-yl)methyl]-7-phenyl-[1,2,4]triazolo[4,3-c]pyrimidin-3-one;
5-amino-8-[2-(hydroxymethyl)-6-methyl-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methyl-4-pyridyl]-2-(oxazol-4-ylmethyl)-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methyl-4-pyridyl]-2-[(1-methylimidazol-2-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(1-methylpyrazol-3-yl)methyl]-[1,2,4]triazolo[4,3-c]pyrimidin-3-one;
5-amino-2-[(2,5-dimethyloxazol-4-yl)methyl]-7-(4-fluorophenyl)-8-[2-(hydroxymethyl)-6-methyl-4-pyridyl]-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-phenyl-[1,2,4]triazolo[4,3-c]pyrimidin-3-one;
5-amino-8-[2-chloro-6-(hydroxymethyl)-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-8-[2-chloro-6-(hydroxymethyl)-4-pyridyl]-7-(4-fluorophenyl)-2-[(1-methylimidazol-2-yl)methyl]-[1,2,4]triazolo[4,3-c]pyrimidin-3-one;
5-amino-8-[2-chloro-6-(hydroxymethyl)-4-pyridyl]-7-(4-fluorophenyl)-2-[(5-methyloxazol-4-yl)methyl]-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-phenyl-[1,2,4]triazolo[4,3-c]pyrimidin-3-one;
5-amino-8-[2-(hydroxymethyl)-6-methoxy-4-pyridyl]-2-[(1-methylimidazol-2-yl)methyl]-7-(2,3,4,5,6-pentadeuteriophenyl)-[1,2,4]triazolo[4,3-c]pyrimidin-3-one;
5-amino-8-[2-(hydroxymethyl)-6-methoxy-4-pyridyl]-2-[(5-methyloxazol-4-yl)methyl]-7-(2,3,4,5,6-pentadeuteriophenyl)-[1,2,4]triazolo[4,3-c]pyrimidin-3-one;
5-amino-7-(4-fluorophenyl)-8-[2-(hydroxymethyl)-6-methoxy-4-pyridyl]-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methoxy-4-pyridyl]-2-[(1-methylimidazol-2-yl)methyl]-[1,2,4]triazolo[4,3-c]pyrimidin-3-one;
5-amino-8-[2-(hydroxymethyl)-6-(trifluoromethyl)-4-pyridyl]-2-[(5-methyloxazol-4-yl)methyl]-7-phenyl-[1,2,4]triazolo[4,3-c]pyrimidin-3-one;
5-amino-8-[2-(hydroxymethyl)-6-(trifluoromethyl)-4-pyridyl]-2-[(1-methylimidazol-2-yl)methyl]-7-phenyl-[1,2,4]triazolo[4,3-c]pyrimidin-3-one;
tert-butyl 3-[5-amino-8-(2,6-dimethyl-4-pyridyl)-3-oxo-7-phenyl-[1,2,4]triazolo[4,3-c]pyrimidin-2-yl]pyrrolidine-1-carboxylate;
5-amino-8-(2,6-dimethyl-4-pyridyl)-7-phenyl-2-pyrrolidin-3-yl-[1,2,4]triazolo[4,3-c]pyrimidin-3-one;
5-amino-8-(2,6-dimethyl-4-pyridyl)-2-(1-methylpyrrolidin-3-yl)-7-phenyl-[1,2,4]triazolo[4,3-c]pyrimidin-3-one;
tert-butyl 2-[[5-amino-8-(2,6-dimethyl-4-pyridyl)-3-oxo-7-phenyl-[1,2,4]triazolo[4,3-c]pyrimidin-2-yl]methyl]pyrrolidine-1-carboxylate;
5-amino-8-(2,6-dimethyl-4-pyridyl)-7-phenyl-2-(pyrrolidin-2-ylmethyl)-[1,2,4]triazolo[4,3-c]pyrimidin-3-one;
5-amino-8-(2,6-dimethyl-4-pyridyl)-2-[(1-methylpyrrolidin-2-yl)methyl]-7-phenyl-[1,2,4]triazolo[4,3-c]pyrimidin-3-one;
5-amino-8-(2,6-dimethyl-4-pyridyl)-2-[[(2R)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-methylpyrrolidin-2-yl]methyl]-7-phenyl-[1,2,4]triazolo[4,3-c]pyrimidin-3-one;
5-amino-8-(2,6-dimethyl-4-pyridyl)-2-[[(2S)-1-(2-methoxyethyl)pyrrolidin-2-yl]methyl]-7-phenyl-[1,2,4]triazolo[4,3-c]pyrimidin-3-one;
5-amino-8-(2,6-dimethyl-4-pyridyl)-2-[[(2R)-1-(2-methoxyethyl)pyrrolidin-2-yl]methyl]-7-phenyl-[1,2,4]triazolo[4,3-c]pyrimidin-3-one;
5-amino-2-[[(2S)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoropyrrolidin-2-yl]methyl]-8-(2,6-dimethyl-4-pyridyl)-7-phenyl-[1,2,4]triazolo[4,3-c]pyrimidin-3-one;
5-amino-2-[[(2S)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-[[(2R)-4,4-difluoro-1-methyl-pyrrolidin-2-yl]methyl]-8-(2,6-dimethyl-4-pyridyl)-7-phenyl-[1,2,4]triazolo[4,3-c]pyrimidin-3-one;
5-amino-2-(2-amino-1-tetrahydrofuran-3-yl-ethyl)-8-(2,6-dimethyl-4-pyridyl)-7-phenyl-[1,2,4]triazolo[4,3-c]pyrimidin-3-one;
5-amino-8-(2,6-dimethyl-4-pyridyl)-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[[(2S)-tetrahydrofuran-2-yl]methyl]-[1,2,4]triazolo[4,3-c]pyrimidin-3-one;
5-amino-8-(2,6-dimethyl-4-pyridyl)-2-[[(2R,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4R)-4-fluoropyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxypyrrolidin-2-yl]methyl]-7-phenyl-[1,2,4]triazolo[4,3-c]pyrimidin-3-one;
5-amino-8-(2,6-dimethyl-4-pyridyl)-2-[[(2R,4R)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-2-[[(2S,4S)-4-hydroxy-1-methyl-pyrrolidin-2-yl]methyl]-7-phenyl-[1,2,4]triazolo[4,3-c]pyrimidin-3-one;
5-amino-8-(2,6-dimethyl-4-pyridyl)-7-phenyl-2-(2-pyrrolidin-1-ylethyl)-[1,2,4]triazolo[4,3-c]pyrimidin-3-one;
5-amino-8-(2-methoxy-6-methyl-4-pyridyl)-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2S)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methyl-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methyl-4-pyridyl]-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-phenyl-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-8-[2-chloro-6-(hydroxymethyl)-4-pyridyl]-7-(4-fluorophenyl)-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-phenyl-2-[[(2R)-tetrahydrofuran-2-yl]methyl]-[1,2,4]triazolo[4,3-c]pyrimidin-3-one;
5-amino-8-[2-(hydroxymethyl)-6-methoxy-4-pyridyl]-7-(2,3,4,5,6-pentadeuteriophenyl)-2-[[(2R)-tetrahydrofuran-2-yl]methyl]-[1,2,4]triazolo[4,3-c]pyrimidin-3-one;
5-amino-7-(4-fluorophenyl)-8-[2-(hydroxymethyl)-6-methoxy-4-pyridyl]-2-[[(2R)-tetrahydrofuran-2-yl]methyl]-[1,2,4]triazolo[4,3-c]pyrimidin-3-one;
5-amino-8-[2-(hydroxymethyl)-6-(trifluoromethyl)-4-pyridyl]-7-phenyl-2-[[(2R)-tetrahydrofuran-2-yl]methyl]-[1,2,4]triazolo[4,3-c]pyrimidin-3-one;
5-amino-8-(2,6-dimethyl-4-pyridyl)-7-phenyl-2-[2-[2-(2-thienyl)pyrrolidin-1-yl]ethyl]-[1,2,4]triazolo[4,3-c]pyrimidin-3-one;
5-amino-8-(2,6-dimethyl-4-pyridyl)-7-phenyl-2-[2-[[1-(pyridine-3-carbonyl)pyrrolidin-3-yl]amino]ethyl]-[1,2,4]triazolo[4,3-c]pyrimidin-3-one;
5-amino-8-(2,6-dimethyl-4-pyridyl)-2-[2-[methyl(1H-pyrazol-4-yl)amino]ethyl]-7-phenyl-[1,2,4]triazolo[4,3-c]pyrimidin-3-one;
5-amino-2-[[1-[[2-(aminomethyl)phenyl]methyl]pyrrolidin-2-yl]methyl]-8-(2,6-dimethyl-4-pyridyl)-7-phenyl-[1,2,4]triazolo[4,3-c]pyrimidin-3-one; and
5-amino-8-(2,6-dimethyl-4-pyridyl)-2-[(5-methyloxazol-4-yl)methyl]-7-(1-piperidyl)-[1,2,4]triazolo[4,3-c]pyrimidin-3-one.

12. The compound of claim 11, or a pharmaceutically acceptable of thereof, selected from the group consisting of:
5-amino-8-(2,6-dimethyl-1-oxido-pyridin-1-ium-4-yl)-7-(4-fluorophenyl)-2-[(5-methyloxazol-4-yl)methyl]-[1,2,4]triazolo[4,3-c]pyrimidin-3-one;
5-amino-7-(4-fluorophenyl)-8-[2-(hydroxymethyl)-6-methyl-4-pyridyl]-2-[(5-methyloxazol-4-yl)methyl]-[1,2,4]triazolo[4,3-c]pyrimidin-3-one; and
5-amino-8-[2-(hydroxymethyl)-6-methyl-4-pyridyl]-7-phenyl-2-[[(2R)-tetrahydrofuran-2-yl]methyl]-[1,2,4]triazolo[4,3-c]pyrimidin-3-one.

13. A pharmaceutical composition comprising a compound of any one of claims 1 to 12, or a pharmaceutically acceptable salt thereof, and a pharmaceutically acceptable carrier, diluent, or excipient.

14. Use of a compound of any one of claims 1 to 12, or a pharmaceutically acceptable salt thereof, for the treatment of a disease or condition mediated by the adenosine receptor.

15. The use of claim 14, wherein the disease or condition mediated by the adenosine receptor is lung cancer, pancreatic cancer, prostate cancer, ovarian cancer, cervical cancer, colorectal cancer, breast cancer, brain cancer, gastric cancer, liver cancer, renal cancer, endometrial cancer, thyroid cancer, bladder cancer, glial cancer, melanoma, or other solid tumor.
